(19) United States
(12) Patent Application Publication
(10) Pub. No.: US 2023/0348983 A1
(43) Pub. Date: Nov. 2, 2023

(54) BIOMARKERS

(71) Applicants: Cambridge Enterprise Limited, Cambridge Cambridgeshire (GB); Genome Research Limited, London Greater London (GB)

(72) Inventors: Matthew Hoare, Cambridge Cambridgeshire (GB); Peter Campbell, Hinxton Cambridgeshire (GB); Stanley NG, Hinxton Cambridgeshire (GB)

(73) Assignees: Cambridge Enterprise Limited, Cambridge Cambridgeshire (GB); Genome Research Limited, London Greater London (GB)

(21) Appl. No.: 18/027,874

(22) PCT Filed: Sep. 23, 2021
(86) PCT No.: PCT/GB2021/052469

§ 371 Date: Mar. 22, 2023

(30) Foreign Application Priority Data
Sep. 23, 2020 (GB) 2015056.1

(51) Int. Cl. C12Q 1/6883

(52) U.S. Cl. CPC C12Q 1/6883; C12Q 2600/118; C12Q 2600/156; C12Q 2600/106

(57) ABSTRACT

The present invention concerns methods of diagnosing and/or prognostication of non-alcoholic fatty liver disease (NAFLD) or alcohol-related fatty liver disease (ARLD) in a subject, wherein said methods comprise detecting somatic mutations in DNA, RNA and/or protein that confer a selective advantage on one or more liver cells of the subject. The present invention also provides methods for identifying subjects suffering from NAFLD or ARLD who would benefit from treatment with a therapeutic agent and/or identifying subjects suffering from NAFLD or ARLD who would benefit from increased disease monitoring. The present invention also provides therapeutic agents that find utility in the treatment of NAFLD or ARLD.

### INTRODUCTION

The present invention relates to methods of diagnosing and/or prognostication of non-alcoholic fatty liver disease (NAFLD) or alcohol-related fatty liver disease (ARLD) in a subject. The present invention also relates to methods for identifying subjects suffering from NAFLD or ARLD who would benefit from treatment with a therapeutic agent and/or identifying subjects suffering from NAFLD or ARLD who would benefit from increased disease monitoring. Also provided herein are therapeutic agents that find utility in the treatment of NAFLD or ARLD.

### BACKGROUND OF THE INVENTION

Non-alcoholic fatty liver disease (NAFLD), estimated to affect one quarter of the world's population, is a syndrome of hepatic inflammation and damage that can progress to non-alcoholic steatohepatitis (NASH), liver fibrosis, cirrhosis, hepatocellular carcinoma (HCC), and liver failure. It is estimated that approximately 10% to 30% of patients with NAFLD develop NASH and approximately 25% to 40% of patients with NASH go on to develop liver fibrosis and cirrhosis which is associated with poor long-term prognosis (Dyson et al., Frontline Gastroenterology 2014; 5:211-218). Studies have shown that patients with NAFLD also have a higher risk of developing liver cancers and gastrointestinal type cancers (Aliens et al., J Hepatol. 2019; 71(6):12294236).

The early stages of NAFLD are characterised by the formation of hepatic steatosis which may go unnoticed until the disease progresses and eventually causes more serious liver damage. As such, the early stages of NAFLD are often only detected by the incidental detection of abnormal liver enzymes in the blood following routine blood testing. Once NAFLD is suspected, imaging technique such as ultrasound are often used in the initial investigation to confirm the presence of hepatic steatosis in the liver. However, to definitively diagnose NAFLD it is frequently necessary to carry out invasive liver biopsies to confirm the presence of hepatic steatosis in the liver tissue. This is associated with risk of patient harm and therefore only used in specific cases. Therefore, in most cases, assessment of whether the patient has NAFLD and whether it is progressive is indirect, through the use of imaging and blood tests.

The identification of disease specific biomarkers, such as disease specific mutations at the genetic and/or protein level, has shown promise for reliably diagnosing and prognosing various diseases and conditions, in particular cancers. In this regard, inherited genetic variations (i.e. germline polymorphisms) in genes associated with insulin resistance or lipid synthesis have been suggested to have minor influences on the inherited risk of developing liver diseases such as NAFLD (see for example, Valenti et al., Journal of Hepatology, 2009, 50, S265 and Hakim et al, Hepatology, 2021, doi: 10.1002/hep. 2038). However, as the development of NAFLD and ARLD is primarily driven by lifestyle and environmental factors, the detection of inherited genetic variations provides only limited insight into the condition of a patient's liver. The presence of somatic mutations in liver cells of patients suffering from severe forms of liver diseases such as cirrhosis and hepatocellular carcinoma (HCC) has also been reported (see, for example, Zhu et al., Cell. 2019; 177(3):608-621, Kim et al., J Gastroenterol. 2019; 54(7):628-640, Brunner et al., Nature, 2019, 574, 538-542, Torrecilla et al., J Hepatol. 2017; 67(6): 1222-1231, and Nault et al., Hepatology. 2014; 60(6):1983-92).

Despite the impact of NAFLD on the health and life expectancy of a significant proportion of the human population, there are currently no reliable diagnostic or prognostic tests that identify patients with NAFLD and those likely to develop more severe forms of liver disease, such as cirrhosis and HCC. Remarkably, there are also no specific treatments available for the treatment of NAFLD. Treatment is often limited to the use of exercise and diet programs and/or treatments to reduce associated risk factors such as obesity, Type 2 diabetes mellitus, high blood pressure and high cholesterol.

The early stages of alcohol-related fatty liver disease (ARLD), although having a different aetiology, have similar pathophysiology to NAFLD. As for NAFLD, there are no also reliable diagnostic or prognostic tests for identifying patients with the early stages of ARLD, nor are there specific treatments available for the treatment of ARLD.

Thus, there remains the need for more reliable diagnostic and prognostic methods that enable the early diagnosis of NAFLD or ARLD and the identification of subjects who are at risk of developing more severe forms of liver disease. There is also a need for treatments that are effective at treating and/or preventing NAFLD or ARLD.

### SUMMARY OF THE INVENTION

The present invention provides a method for diagnosing and/or prognostication of non-alcoholic fatty liver disease (NAFLD) or alcohol-related fatty liver disease (ARLD) in a subject, said method comprising:
a) providing a biological sample comprising DNA, RNA and/or protein derived from one or more liver cells of the subject;
b) detecting a somatic mutation in the DNA, RNA and/or protein that confers a selective advantage on the liver cell, wherein the presence of a somatic mutation that confers a selective advantage on the liver cell indicates that the subject is suffering from NAFLD or ARLD, is at risk of developing NAFLD and/or ARLD, is at risk of developing a more severe form of liver disease, and/or is at risk of developing a disease or condition associated with liver disease.

The present invention also provides a method for diagnosing and/or prognostication of non-alcoholic fatty liver disease (NAFLD) in a subject, said method comprising:
a) providing a biological sample obtained from the subject; and
b) detecting one or more somatic mutations in the FOXO1 and/or GPAM genes in the biological sample, wherein the presence of one or more somatic mutations indicates that the subject is suffering from NAFLD, is at risk of developing NAFLD, is at risk of developing a more severe form of liver disease and/or is at risk of developing a disease or condition associated with liver disease.

The present invention also provides a method for identifying a subject suffering from NAFLD or ARLD who would benefit from treatment with a therapeutic agent that inhibits or modulates FOXO1, GPAM, CIDEB, ACVR2A, ALB or TNRC6B protein activity and/or identifying a subject suffering from NAFLD or ARLD who would benefit from increased disease monitoring. Also disclosed herein are therapeutic agents for use in the treatment of NAFLD or ARLD, wherein the therapeutic agent is one that inhibits or modulates FOXO1, GPAM, CIDEB, ACVR2A, ALB or TNRC6B protein activity, and methods of treating or preventing NAFLD or ARLD by administering to a subject a therapeutic agent that inhibits or modulates FOXO1, GPAM, CIDEB, ACVR2A, ALB or TNRC6B protein activity.

As described herein, the present inventors analysed somatic mutations from 1590 genomes across 34 liver samples, including alcohol-related fatty liver disease (ARLD), non-alcoholic fatty liver disease (NAFLD) and normal controls (i.e. liver samples from healthy subjects). From this analysis the present inventors discovered that the occurrence of somatic mutations that confer a selective advantage on a liver cell (mutations which can be considered akin to a driver mutation in cancer) is central to the development of NAFLD and ARLD in a subject. The present inventors have found that the (direct or indirect) detection, quantification and/or monitoring of such somatic mutations in liver cells of a subject is particularly useful for diagnostic and prognostic testing of NAFLD and ARLD. In particular, in patients with NAFLD or ARLD, the present inventors identified various mutations in the FOXO1, GPAM, CIDEB, ACVR2A, ALB and TNRC6B genes. Notably, the present inventors have found that the mutations identified in the FOXO1, GPAM, CIDEB, ACVR2A, ALB and TNRC6B genes can be used as markers for NAFLD or ARLD to enable early diagnosis and prognosis of NAFLD or ARLD. Furthermore, the FOXO1, GPAM, CIDEB, ACVR2A, ALB and TNRC6B mutations described herein also provide novel molecular targets for the treatment and/or prevention of NAFLD or ARLD.

The present invention also provides an in vitro diagnostic kit for use in the diagnosis and/or prognosis of NAFLD or ARLD in a subject, said kit comprising one or more reagents for detecting one or more somatic mutations in one or more genes selected from the group consisting of FOXO1, GPAM, CIDEB, ACVR2A, ALB and TNRC6B, and optionally detecting one or more somatic mutations in the CLCN5 gene, NEAT1 gene and/or measuring telomere length.

For the avoidance of doubt, the terms “alcohol-related liver disease” and “ARLD” as used herein refer to liver damage in a subject that is caused by excessive alcohol consumption. As for NAFLD, the early stages of ARLD are typically characterised by the formation of hepatic steatosis. The formation of hepatic steatosis caused by excessive alcohol consumption may also be referred to as alcoholic fatty liver disease (AFLD). ARLD may also be associated with alcoholic hepatitis and cirrhosis. AFLD may therefore be considered a subgenus of ARLD.

Also for the avoidance of doubt, throughout the present application, the term ARLD should be considered to incorporate the term AFLD. Accordingly, the present invention also concerns methods for diagnosing and/or prognostication of NAFLD or AFLD in a subject, methods for identifying subjects suffering from NAFLD or AFLD who would benefit from treatment with a therapeutic agent, methods for identifying subjects suffering from NAFLD or AFLD who would benefit from increased disease monitoring, and therapeutic agents that find utility in the treatment of NAFLD or AFLD.

### DESCRIPTION OF THE DRAWINGS

FIG. 1 shows an overview of the hierarchical experimental design used to investigate the convergent FOXO1 mutations acquired in chronic liver disease. 34 livers for normal control, ARLD and NAFLD were sampled (1 sample/liver for 32 livers and 8 samples/liver for 2 livers). These samples underwent laser capture microdissection to generate 21-52 microdissections/sample, each of which was individually whole genome sequenced (1590 whole genomes overall).

FIG. 2A shows the distribution of somatic mutations in FOXO1 grouped by microdissections from affected patients. The pie charts show the fraction of sequencing reads reporting the mutant allele in each microdissection. The hotspot S22 residue is within a canonical, highly conserved motif for binding by 14-3-3 nuclear export proteins and phosphorylation by AKT1 and AMPK.

FIG. 2B relates to the FOXO1 mutations in PD37239, and FIG. 2C relates to the FOXO1 mutations in PD37918, both patients with NAFLD. The left panels of FIG. 2B and FIG. 2C show the phylogenetic trees, with darker lined branches showing independently acquired mutations. Solid lines indicate that nesting is in accordance with the pigeonhole principle; dashed lines indicate that nesting is in accordance with the pigeonhole principle, assuming that hepatocytes represent <100% of cells. The right panels show the clones from the phylogenetic trees mapped onto a haematoxylin and eosin (H&E)-stained light micrograph of the patient's liver biopsy, with FOXO1-mutant clones shaded to match the darker lined branches shown on the phylogenetic trees.

FIG. 2D shows a chromothripsis event affecting chromosome 13 in one of the microdissections from PD37907, a patient with NAFLD. Black points represent corrected read depth along the chromosome. Lines and arcs represent structural variants. The structural variant that breaks FOXO1 is highlighted (labelled “Rearranges FOXO1” in the figure), and would be predicted to break the gene within the first intron, preserving the first coding exon but deleting the remaining coding exons.

FIG. 2E shows the clone map of FIG. 2B laid onto a H&E-stained section of PD37239. On the left of FIG. 2E, raw sequencing data from representative samples with and without FOXO1 mutations are shown, with their physical locations on the H&E section shown by the arrows. The locations of the S22W and R21L mutations are marked with arrows. The scatterplots arranged around the H&E section represent variant allele fraction plots of mutations in pairs of samples. The grey-scale shades of the x and y axis titles match the clone map grey-scale shades of the H&E section. In clonally related pairs of samples, most of the mutations are shared by both samples, evident as a cloud of mutations with non-zero variant allele fraction. In clonally unrelated samples, the mutations line the x and y axes, with the one exception being the FOXO1 mutation, indicating that it is independently acquired in the two clones.

FIG. 3 shows phylogenetic trees and clone maps for PD37234 (FIG. 3A), PD37105 (FIG. 3B) and PD37245 (FIG. 3C). The left panel of each figure shows the phylogenetic tree, with darker lined branches showing independently acquired mutations. Solid lines indicate that nesting is in accordance with the pigeonhole principle; dashed lines indicate that nesting is in accordance with the pigeonhole principle, assuming that hepatocytes represent <100% of cells. The right panel shows the clones from the phylogenetic tree mapped onto an H&E-stained photomicrograph of the liver, with FOXO1-mutant clones shaded to match the darker lined branches shown on the phylogenetic trees.

FIG. 4A shows the distribution of somatic mutations in CIDEB in chronic liver disease. Amino acid residues are coloured by type, with observed mutations in chronic liver disease shown above the wild-type protein sequence. FIG. 4B shows the CIDEB mutations in one of the Couinaud segments analysed from PD48637, a patient with NAFLD. The left panel shows the phylogenetic tree, with darker lined branches showing independently acquired mutations. Solid lines indicate that nesting is in accordance with the pigeonhole principle; dashed lines indicate that nesting is in accordance with the pigeonhole principle, assuming that hepatocytes represent <100% of cells. The right panel shows the clones from the phylogenetic tree mapped onto an H&E-stained photomicrograph of the liver, with CIDEB-mutant clones shaded to match the darker lined branches shown on the phylogenetic trees. One clone had two independent point mutations in CIDEB, on different alleles (compound heterozygosity). FIG. 4C shows a further example of CIDEB mutations in patients with chronic liver disease. Phylogenetic trees and clone maps are shown for one of the Couinaud segments of PD48367 with CIDEB mutations.

FIG. 5A shows the distribution of somatic mutations in GPAM according to genomic location. Pie charts show the fraction of sequencing reads reporting the mutant allele in each microdissection. Multiple sequence alignments showing the evolutionary conservation of each mutated residue ±3 amino acids are shown for representative species.

FIG. 5B shows a tandem duplication upstream of GPAM in a microdissection from PD37110, a patient with ARLD. GPAM is left intact, but the tandem duplication starts 20 kb upstream of the gene.

FIG. 5C shows the GPAM mutations in PD37231, a patient with ARLD. The left panel shows the phylogenetic tree, with darker lined branches showing independently acquired mutations. Solid lines indicate that nesting is in accordance with the pigeonhole principle; dashed lines indicate that nesting is in accordance with the pigeonhole principle, assuming that hepatocytes represent <100% of cells. The right panel shows the clones from the phylogenetic tree mapped onto an H&E-stained photomicrograph of the liver, with GPAM-mutant clones shaded to match the darker lined branches shown on the phylogenetic trees.

FIGS. 5D and 5E show further examples of GPAM mutations in patients with ARLD. Phylogenetic trees and clone maps are shown for one of the Couinaud segments of PD37111 (FIG. 5D) and one of the Couinaud segments of PD37232 (FIG. 5E).

FIG. 6A shows the distribution of somatic mutations in ACVR2A according to genomic location. Pie charts show fraction of sequencing reads reporting the mutant allele in each microdissection. FIG. 6B shows two microdissections in different patients showing structural variants generating copy loss of ACVR2A. Black points represent corrected read depth along the chromosome. Lines and arcs represent structural variants.

FIG. 7 shows the distribution of somatic mutations in CLCN5 according to genomic location. Pie charts show the fraction of sequencing reads reporting the mutant allele in each microdissection.

FIG. 8 shows the distribution of somatic mutations in the long non-coding RNA, NEAT1, according to genomic location. Pie charts show fraction of sequencing reads reporting the mutant allele in each microdissection.

FIG. 9 shows the distribution of somatic mutations in TNRC6B according to genomic location. Pie charts show fraction of sequencing reads reporting the mutant allele in each microdissection.

FIG. 10A shows the live cell imaging of HepG2 cells transfected with the indicated wild-type or mutant constructs of FOXO1 fused with a C-terminal eGFP. Cells were counterstained with nuclear (Hoechst 33342) and cytoplasmic (SPY-555-Actin) markers. Live-cell imaging was conducted after overnight serum starvation and then stimulation with 100 nM insulin. FIG. 10B shows the quantification of the eGFP localisation, expressed as log nuclear-cytoplasmic fluorescence ratio (mean±SEM) during live cell imaging (wild-type cells, n 6186 and FOXO1S22W cells, n ±7172 per time point). FIG. 10C shows a heat map of the concentrations of metabolites (columns) measured in HepG2 cells measured across 4 conditions (wild-type FOXO1 construct, with or without insulin; S22W FOXO1 construct, with or without insulin) in 5 replicates each (rows). Shown in the figure are the 43 metabolites that were significantly different between mutant and wild-type constructs after correction for multiple hypothesis testing (q<0.01), with intermediates from the pentose phosphate and glycolysis/gluconeogenesis pathways highlighted in bold (i.e. hexose-phosphate, dihydroxyacetone-phosphate, pentose-phosphates, sedoheptulose 7-phosphate, glycerol-3-phosphate and glyceraldehyde-3-phosphate).

FIG. 10D shows HepG2 cells following transfection with the indicated wild-type or mutant constructs of FOXO1 fused with a C-terminal GFP. Cells were counterstained with DAPI to highlight the nucleus, and imaged after overnight serum starvation conditions (left) and after 15 minutes of exposure to 100 nM insulin (right).

FIG. 10E shows the wide-field view of the entire coverslip of HepG2 cells pseudocoloured on a scale by the nuclear-cytoplasmic ratio of FOXO1-GFP. Cells were imaged under conditions of serum starvation (left), after exposure to insulin 100 nM for 15 minutes (middle) or 5% foetal calf serum (FCS) for 15 minutes (right).

FIGS. 10F and 10G show the nuclear-cytoplasmic ratios for wild-type and mutant FOXO1-GFP constructs in HCC cell lines. Wide-field views of Hep3B (FIG. 11A) and PLC/PRF5 (FIG. 11B) cells pseudocoloured on a scale by the nuclear-cytoplasmic ratio of FOXO1-GFP are shown. The cells were imaged under conditions of serum starvation (left), after exposure to insulin 100 nM for 15 minutes (middle) of foetal calf serum (FCS) for 15 minutes (right).

FIG. 10H shows an immunoblot of HepG2 cells expressing ectopic eGFP-tagged wild-type or mutant FOXO1 constructs as indicated and treated for 15 minutes with vehicle or insulin (100 nM). The cells were analysed for the indicated proteins by immunoblotting. Molecular weight markers (kDa) indicated.

FIG. 11A shows a heatmap of the gene expression levels for genes in the ‘Canonical Glycolysis’ gene set from Gene Ontology (GO), http://geneontology.org (GO:0061621). The order of genes on the x axis is determined by the level of significance (and direction of change) and the order of samples on they axis is by condition (FOXO1 status and insulin status). FIG. 11B shows a heatmap of the gene expression levels for genes in the ‘Cell cycle, mitotic’ gene set from Reactome (R-HSA-69278—https://reactome.org/). The order of genes on the x axis is determined by the level of significance (and direction of change) and the order of samples on the y axis is by condition (FOXO1 status and insulin status). FIGS. 11C, D and E show enrichment plots for the ‘FOXO-mediated transcription of oxidative stress, metabolic and neuronal genes’ gene set of Reactome (9615017—https://reactome.org/) (FIG. 11C); ‘Lipid catabolic process’ gene set of GO (0016042) (FIG. 11D); and ‘Apoptotic process’ gene set of GO (0006915) (FIG. 11E). In each, the top panel reflects the cumulative enrichment score as the gene set is traversed from most up-regulated to most down-regulated in the presence of FOXO1-mutant constructs. The bottom panel in each shows the ranking of each gene in the gene set across all genes measured.

FIG. 12A shows a stacked bar chart showing the estimated cumulative liver mass carrying driver mutations, extrapolated from samples analysed in each patient. The calculations assume a total liver mass of 1500 g for each patient. Bars are hatched for each of the 6 recurrently mutated genes identified, and patient codes on the x axis are coloured for disease status. FIG. 12B shows the estimated clone size for the 4 most frequently mutated genes (FOXO1, CIDEB, GPAM and ACVR2A) compared to wild-type clones. The points are overlaid on box-and-whisker plots where the median is marked with a heavy black line and the interquartile range in a thin black box. The whiskers mark the full range of the data or 25^{th}/75^{th} centile plus 1.5× the interquartile range (whichever is smaller). FIG. 12C shows a scatter plot of the distribution of ages of patients in the cohort by whether they carried clones with mutations in the specified genes or not. FIG. 12D shows stacked bar charts of the proportion of patients with or without type 2 diabetes by whether they carried driver mutations in each gene. FIG. 12E shows stacked bar charts of the distribution of the NAFLD Activity Score (NAS) by whether they carried driver mutations in each gene, with low scores denoting a low degree of histological abnormality.

FIGS. 13A and 13B show scatter plot of the distribution of telomere lengths (y axis) by patient (x axis). Each point represents the average telomere length estimated from genome sequencing data for the constituent microdissection with the highest median variant allele fractions (VAF) in each clone within that patient. In FIG. 13A the patients are ordered on the x axis and marked by disease status (block=normal/healthy; blank=ARLD; lines=NAFLD); within each disease entity, patients are ordered by ascending age. In FIG. 13B the points are overlaid on box-and-whisker plots where the median is marked with a heavy black line and the interquartile range in a thin black box. The whiskers denote mark the full range of the data or 25^{th}/75^{th} centile plus 1.5× the interquartile range (whichever is smaller).

FIGS. 14A and 14B each show the telomere lengths layered onto two representative phylogenetic trees from ARLD (FIG. 14A) and NAFLD (FIG. 14B). Branches are shaded on a grey-scale according to telomere lengths of the microdissection with the highest median variant allele fraction assigned to that branch. The internal nodes are estimated using maximum likelihood and the scale was interpolated along each branch. FIG. 14C shows further examples of phylogenetic trees shaded by telomere lengths. Telomere lengths layered onto two representative phylogenetic trees from normal liver (top), ARLD (middle) and NAFLD (bottom). Branches are shaded on a grey-scale according to telomere lengths of the sample with the highest variant allele fraction assigned to that branch. The internal nodes are estimated using maximum likelihood and the scale was interpolated along each branch.

FIG. 15 shows posterior distributions of the effect size of clone size (per log₁₀(μm²)), age (per decade of life) and disease state (NAFLD and ARLD versus normal) on telomere lengths. Density plots are shown from the MCMC (Markov Chain Monte Carlo) sampler, shaded by decile.

FIG. 16A shows details of a new mutational signature that was noted by the inventors. In FIG. 16B, there is shown the variability in activity between nearby clones within the same liver sample.

FIGS. 16C, 16D, 16E and 16F show 3 hepatic resection samples from one patient over a 5-year timespan.

In FIGS. 16G, 16H and 161, there are shown the distribution of the signatures in samples of normal liver cells, ARLD-affected cells, NAFLD-affected cells and in 2 patients with NAFLD with all 8 anatomic segments sampled.

FIG. 17 shows an overall HDP node structure including the concentration parameter settings used for signature extraction.

### DETAILED DESCRIPTION OF THE INVENTION

As described herein, the present inventors have developed a method for diagnosing and/or prognostication of non-alcoholic fatty liver disease (NAFLD) or alcohol-related fatty liver disease (ARLD) in a subject which involves detecting the occurrence of somatic mutations that confer a selective advantage on one or more liver cells in a subject. Accordingly, the present invention provides a method for diagnosing or prognostication of NAFLD or AFLD in a subject, said method comprises the steps of
a) providing a biological sample comprising DNA, RNA and/or protein derived from one or more liver cells of the subject; and
b) detecting a somatic mutation in the DNA, RNA and/or protein that confers a selective advantage on the liver cell, wherein the presence of a somatic mutation indicates that the subject is suffering from NAFLD or ARLD, is at risk of developing NAFLD or ARLD, is at risk of developing a more severe form of liver disease and/or is at risk of developing a disease or condition associated with liver disease such as gastrointestinal cancer. In particular embodiments, the detection of a somatic mutation that confers a selective advantage on the liver cell indicates that the subject is suffering from NAFLD or ARLD, is at risk of developing NARLD or ARLD, and/or is at risk of developing non-alcoholic steatohepatitis (NASH), liver fibrosis, liver cirrhosis, cancer, for example hepatocellular carcinoma (HCC), or liver failure. In exemplary embodiments, the method of diagnosing or prognostication of NAFLD or ARLD described herein is an in vitro method of diagnosing or prognostication of NAFLD or ARLD.

For the avoidance of doubt, the term “prognostication” as used herein refers to the process of estimating/predicting the likely course and outcome of NAFDL or ARLD in a subject, and/or chance that a subject has of recovering from NAFDL or ARLD. For example, a subject whose liver disease is not regressing in response to certain treatment(s), as determined, for example by using a method of the present invention, may be considered to have a poor prognosis.

a) Providing a Biological Sample Comprising DNA, RNA and/or Protein Derived from One or More Liver Cells of the Subject:

Typically, the biological sample comprising DNA, RNA and/or protein derived from one or more liver cells is obtained from a blood sample, urine sample or tissue sample obtained from a subject. A blood sample may be a blood serum, blood plasma or whole blood sample obtained from a subject. A tissue sample may be a biopsy sample, in particular a liver biopsy sample obtained from a subject. A liver biopsy sample comprises liver cells, such as hepatocytes (HCs), hepatic stellate cells (HSCs), Kupffer cells (KCs) or liver sinusoidal endothelial cells (LSECs). Preferably, the liver biopsy sample obtained from the subject comprises hepatocytes.

Typically, the biological sample comprises DNA, RNA and/or protein derived from at least 10, at least 100, or at least 1000, at least 10,000, at least 10⁴, at least 10⁵, or at least 10⁶ liver cells. For example, about 10 to about 10⁶ liver cells, about 100 to about 10⁵, about 500 to about 10⁴, about 10 to about 50,000, or about 1000 to about 50,000 (for example, about 1000 to about 10,000, about 1000 to about 20,000, about 1000 to about 30,000, or about 1000 to about 40,000 liver cells). In exemplary embodiments, the biological sample comprises DNA, RNA and/or protein derived from about 10 to about 50,000 liver cells (for example, about 10 to about 50,000 hepatocyte cells) of the subject.

In certain embodiments wherein the sample comprises DNA and/or RNA derived from one or more liver cells, the sample comprises genomic DNA (gDNA) and/or messenger RNA (mRNA). For example, the sample may comprise DNA or RNA extracted from cells present in a blood sample or biopsy sample obtained from the subject.

In certain embodiments, the biological sample comprising DNA, RNA and/or protein is derived from a liver biopsy sample from the subject. Methods for obtaining liver biopsies from a subject are known in the art and are within the routine abilities of a clinical practitioner. Liver biopsy samples may be obtained from a subject by, for example, percutaneous, transjugular or laparoscopic liver biopsy methods.

In exemplary embodiments, the biological sample comprising DNA, RNA and/or protein is derived from a microdissection of a liver biopsy sample obtained from the subject. For example, a liver biopsy sample may be obtained from a subject and then dissected into about 1 to about 100 (for example, 10, 20, 30, 40, 50, 60, 70, 80, 90 or 100) separate microdissection samples. Typically, a liver biopsy sample is dissected into about 10 to about 60 microdissection samples. Each separate microdissection sample obtained from a liver biopsy sample may comprise about 10 to about 50,000 liver cells (10 to about 50,000 hepatocyte cells). Typically, each separate microdissection sample comprises about 100 to about 500 liver cells.

Methods for extracting DNA and/or RNA from a cell, such as a liver cell, are known in the art. For example, DNA and RNA can be isolated from a cell or tissue sample using reagents to lyse cells followed by a column-based approach and/or a bead-based approach to purify the DNA and/or RNA. Examples of commercially available kits that may be used to isolate DNA or RNA from a cell or tissue sample include, but are not limited to the QIAamp® DNA Kit, the EZ1° DNA Tissue Kit, the Oligotex® Direct mRNA Kit, and the Arcturus® PicoPure® DNA Extraction Kit.

The isolated DNA or RNA may be amplified before analysis. Before amplification, the isolated RNA may be converted to DNA by reverse transcription using a reverse transcriptase. DNA or RNA may be amplified using techniques known in the art, for example by using polymerase chain reaction (PCR)-based methods or reverse transcription polymerase chain reaction (RT-PCR)-based methods. The isolated DNA or RNA may be also be used to construct a DNA or RNA library suitable for the sequencing technique to be used. For example, a DNA library may be constructed using a transposase-based method.

The DNA or mRNA isolated from a sample may also be quantified using methods known in the art such as Quantitative PCR (qPCR) and Quantitative reverse transcription PCR (RT-qPCR). Thus, in certain embodiments, the method may further comprise a step of amplifying and/or quantifying the amount of DNA or RNA obtained from a biological sample obtained from a subject.

In embodiments wherein the biological sample comprises DNA, the DNA may be circulating free DNA (cfDNA). That is to say, that the biological sample provided in step a) of the method of the invention may comprise cfDNA derived from one or more liver cells of a subject. As used herein the term “circulating free DNA” (cfDNA) refers to DNA fragments that have been released into the blood plasma and are found freely circulating in the blood stream, or in the urine. cfDNA is generally double-stranded DNA consisting of small fragments (70 to 200 bp). Accordingly, the biological sample may be any suitable sample known in the art in which cfDNA can be detected and/or isolated. For example, the sample may suitably be a blood sample, a plasma sample, or a urine sample that comprises cfDNA. Methods for identifying liver-derived cfDNA in a biological sample are known in the art, examples of such methods are described in, for example, Jiang et al, J Hepatology, 2019, 71(2), 409-421, Moss et al., Nat Commun, 2018, 9, 5068, and Punia et al., BMC Gastroenterol, 2021, 21, 149-159.

In embodiments wherein the biological sample comprises cfDNA, the method may further comprise isolating the cfDNA from the sample. cfDNA can be isolated from the sample using a variety of techniques known in the art. For example, cfDNA can be isolated by a column-based approach and/or a bead-based approach. In some embodiments, cfDNA is isolated by means of a column-based approach, for example using a commercially available kit such as QIAamp® circulating nucleic acid kit. In some embodiments, cfDNA is isolated by means of a bead-based approach, for example an automated cfDNA extraction system using a commercially available kit such as Maxwell RSC ccfDNA Plasma Kit (Promega).

The isolated cfDNA may be amplified before analysis. Thus, the method may further comprise amplification of the isolated cfDNA. Techniques suitable for amplifying cfDNA are known in the art and include, but are not limited to, cloning, polymerase chain reaction (PCR), polymerase chain reaction of specific alleles (PASA), polymerase chain ligation, nested polymerase chain reaction, and so forth.

In embodiments wherein the biological sample comprises protein, the level of mutated protein may be measured using any suitable technique known in the art. The method may comprise isolating the protein from the sample and then assessing the quantity of the mutated protein present. Depending on the method used and the level of accuracy required, a purification step may be carried out. In some circumstances, simple lysis of cells in the sample may be sufficient. The level of mutated protein present may be assessed using one or more techniques selected from enzyme-linked immunosorbent assay (ELISA), Western Blot analysis and mass spectrometry.

As used herein, a “subject” refers to an animal, including mammals such as humans. Preferably, the subject is a human subject. In embodiments, the subject is known or suspected to have a NAFLD or ARLD, and/or is known or suspected to have a risk of developing NAFLD or ARLD. In certain embodiments, the subject is one that has a disease or condition known to increase the risk of developing NAFLD or ARLD, to increase the risk of developing a more severe form of liver disease and/or to increase the risk of developing a disease or condition associated with liver disease. For example, the subject may be one who is known or suspected to be suffering from gastrointestinal cancer, obesity, type 2 diabetes mellitus, hypertension, dyslipidaemia (e.g. hypercholesterolaemia) and/or cardiovascular disease.

b) Detecting a Somatic Mutation in the DNA, RNA and/or Protein that Confers a Selective Advantage on the Liver Cell Clone:

A somatic mutation is a permanent alteration of the DNA sequence of a gene that is acquired during the lifetime of an individual. That is to say that somatic mutations are not present in the germline DNA of an individual, and are therefore not inherited from a parent like germline polymorphisms. A somatic mutation may occur spontaneously due to infidelity of DNA replication occurring at each cell division creating substitutions, deletions or insertions of nucleotides into the DNA of a cell. A somatic mutation may also be caused by environmental factors such as ultraviolet radiation, chemical exposure or viral infections.

Somatic mutations in a gene may be expressed by a cell to produce a mutated protein wherein one or more nucleotide substitutions in the gene (i.e. each nucleotide substitution being a “single-nucleotide variant” or “SNV”) can result in a different amino acid being coded for compared to the amino acid coded for by the somatic non-mutated nucleic acid sequence, thus resulting in a different amino acid in the protein/peptide compared to the protein/peptide in a normal non-diseased cell (e.g. a healthy liver cell). Nucleotide insertion(s) and/or deletion(s) can result in a reading frame error (i.e. an “indel mutation” or “frameshift mutation”) thus resulting in a new amino acid sequence at the protein level (i.e. nucleotide insertion(s) or deletion(s) altering the reading frame of the DNA and thus altering most or all of the amino acids encoded by the DNA after the mutation compared to a normal cell (e.g. a healthy liver cell)). Additionally, or alternatively, an insertion and/or deletion can result in the introduction of a stop codon, thus resulting in a truncated protein at the protein level (i.e. a nonsense mutation). A nucleotide substitution can individually alter codon(s) and result in amino acid substitution(s) at the protein level and/or the introduction of a stop codon, thus resulting in a truncated protein at the protein level.

The somatic mutation detected in step b) of the method of the invention is one that confers a selective advantage on a liver cell of the subject. The term “selective advantage” as used herein refers to an advantage conferred to a given cell through one or more mutations that enables the cell to survive and/or reproduce (for example, the somatic mutation is one that confers a growth advantage on a liver cells of the subject) better than a cell that does not have the same one or more mutations. For example, a somatic mutation that confers a selective advantage on a liver cell may be one that enables the liver cell to survive under toxic condition caused by the accumulation of lipids within the cell. Mutations that confer a selective advantage on a liver cell may result in the positive selection of the liver cell in a given microenvironment of the liver resulting in dominant liver cell clones comprising liver cells that are able to survive and/or reproduce better than liver cells that do not have the same one or more mutations. Thus, for example, in certain embodiments, the somatic mutation detected in step b) of the method of the invention is one that confers a selective advantage on the liver cell by enabling the liver cell to survive under toxic conditions associated with lipid accumulation in the liver. Additionally, or alternatively, the somatic mutation that confers a selective advantage on the liver cell may be one that is associated with lipid accumulation in the liver, for example, one that is associated with increased lipid accumulation in the liver and/or decreased lipid metabolism in the liver.

For the avoidance of doubt, the term “driver mutation” as used herein also refers to mutations that confer a selective advantage on a cell (e.g. a liver cell) of the subject.

Methods for identifying somatic mutations that confer a selective advantage on a cell, such as liver cell, are known in the art. Exemplary methods are described herein which involve the use of the dN/dScv method described by Martincorena, I. et al., Cell 2017, 171, 1029-1041.e21, and the NBR algorithm described by Rheinbay, E. et al., Nature, 2020, 578, 102-111.

The present inventors have found that the detection of independently acquired somatic mutations that confer a selective advantage in multiple liver cells in a sample derived from a subject's liver provides an especially reliable method for diagnosing and/or prognosing of NAFLD or ARLD in the subject. Thus, in certain embodiments, step a) of the method of the invention comprises providing a biological sample comprising DNA, RNA and/or protein derived from at least 10, at least 100, or at least 1000, at least 10⁵, or at least 10⁶ liver cells. For example, step b) may comprise detecting one or more somatic mutations in a biological sample comprising at least 100, at least 300, at least 400, at least 500, at least 1000 liver cells. In such embodiments, step b) comprises detecting one or more somatic mutations in said biological sample derived from at least 10, at least 100, or at least 1000, at least 10⁵, or at least 10⁶ liver cells, wherein the presence of one or more somatic mutations that confers a selective advantage on the one or more liver cells indicates that the subject is suffering from NAFLD or ARLD, is at risk of developing NAFLD and/or ARLD, is at risk of developing a more severe form of liver disease, and/or is at risk of developing a disease or condition associated with liver disease.

The present inventors have found that the analysis of a biological sample comprising DNA, RNA and/or protein derived from about 1000 to about 50,000 liver cells (for example, hepatocyte cells) provides a particularly reliable snapshot of the genetic landscape of the liver, and/or the progression and/or severity of NAFLD or ARLD in the liver of the subject. Thus, in certain embodiments, step a) of the method of the invention comprises providing a biological sample comprising DNA, RNA and/or protein derived from about 1000 to about 50,000 liver cells (for example, about 1000 to about 50,000 hepatocyte cells) of the subject.

Additionally, or alternatively, step b) of the method of the invention may be repeated using one or more different biological samples obtained from the subject. For example, one or more liver biopsy samples, or microdissection samples thereof, one or more blood samples and/or one or more urine samples. For example, step b) may be repeated with one or more different biological samples comprising DNA, RNA and/or protein derived from liver cells of the subject that were obtained from liver biopsies from different locations in the subject's liver (for example, liver biopsy samples obtained from different lobes of the liver).

Additionally or alternatively, step b) of the method of the invention may be repeated using one or more different biological samples obtained from one liver biopsy sample from the subject. For example, step b) may be repeated with one or more biological samples obtained by microdissecting a liver biopsy sample from the subject into more than one separate microdissection samples.

Analysis of one or more different biological samples, such as one or more different liver biopsies, one or more different microdissection samples thereof, one or more different blood samples, and/or one or more different urine samples, provides wider insight into the genetic landscape of the liver, and/or the progression and/or severity of NAFLD or ARLD throughout the liver of the subject. Thus, in certain embodiments, the method of the invention comprises providing one or more different biological samples comprising DNA, RNA and/or protein derived from liver cells of the subject, and repeating step b) with the one or more different biological samples to determine if the subject is suffering from NAFLD or ARLD, is at risk of developing NAFLD and/or ARLD, is at risk of developing a more severe form of liver disease, and/or is at risk of developing a disease or condition associated with liver disease.

In certain embodiments, the somatic mutation detected in step b) that confers a selective advantage on a liver cell of the subject is within a gene selected from the group consisting of FOXO1, GPAM, CIDEB, ACVR2A, ALB and TNRC6B.

Thus, in certain embodiments, step b) of the method of the invention comprises detecting one or more somatic mutations in the FOXO1 gene that confer a selective advantage on a liver cell of the subject. FOXO1 encodes the major transcription factor downstream of insulin signalling. In the fasting state, without insulin, the FOXO1 protein is active in the nucleus of hepatocytes, up-regulating expression of genes in gluconeogenesis, glycolysis and lipolysis pathways. Upon insulin binding its receptor, AKT is activated through PI3K. AKT subsequently phosphorylates the FOXO1 protein in the nucleus, with the threonine at position 24 of the FOXO1 protein being one of three known AKT phosphorylation targets.

In subjects suffering from NAFLD or ARLD, the present inventors have identified somatic mutations within the FOXO1 gene that impair insulin-mediated nuclear export of the FOXO1 protein. Impairment of FOXO1 function contributes to decreased insulin sensitivity which is highly prevalent in subjects with increased intrahepatic fat. The presence of one or more of these mutations in the FOXO1 gene may indicate that a subject is suffering from NAFLD or ARLD, or that a subject has increased risk of developing NAFLD or ARLD, has an increased risk of developing a more severe form of liver disease and/or has an increased risk of developing a disease or condition associated with liver disease. Thus, in certain embodiments, the method comprises the detection of one or more somatic mutations in the FOXO1 gene that result in an amino acid mutation of the FOXO1 protein that impairs insulin-mediated nuclear export of the FOXO1 protein.

Typically, at least one of the somatic mutations detected in the FOXO1 gene is in the region of the FOXO1 gene that encodes the N-terminal 14-3-3 protein binding motif of the FOXO1 protein (i.e. amino acids 20 to 28 of the FOXO1 protein). The present inventors have found that somatic mutations within the N-terminal 14-3-3 protein binding motif of the FOXO1 protein are particularly indicative of NAFLD or ARLD in a subject. Thus, in preferred embodiments, step b) of the method of the invention comprises detecting one or more somatic mutations in the FOXO1 gene that result in an amino acid mutation within the N-terminal 14-3-3 protein binding motif of the FOXO1 protein.

In preferred embodiments, step b) of the method of the invention comprises detecting one or more somatic mutations that results in a S22W amino acid substitution, a R21L amino acid substitution and/or a S22 nonsense mutation (referred to herein as S22*) in the FOXO1 protein. The present inventors have found that somatic mutations in the FOXO1 gene that result in a S22W amino acid substitution, a R21L amino acid substitution or a S22 nonsense mutation in the FOXO1 protein are especially indicative of NAFLD or ARLD in a subject. Thus, in particularly preferred embodiments, step b) of the method of the invention comprises the detection of one or more somatic mutations in the FOXO1 gene that result in a S22W amino acid substitution in the FOXO1 protein.

The present inventors have also found that somatic mutations within the GPAM gene may be used as an indicator of NAFLD or ARLD in a subject, a further indicator of an increased risk of the subject developing NAFLD or ARLD and/or an indicator of an increased risk of the subject developing a more severe form of liver disease or an associated disease or condition. The GPAM gene encodes the glycerol-3-phosphate acyltransferase 1, mitochondrial protein (referred to herein as the GPAM protein or the GPAT protein). The GPAM protein is an enzyme that catalyses esterification of long chain acyl-CoAs with glycerol-3-phosphate. Typically, the one or more somatic mutation detected in the GPAM gene results in impaired or abrogated function of a GPAM protein, particularly the impairment or abrogation of a GPAM protein's ability to catalyse esterification of long chain acyl-CoAs with glycerol-3-phosphate. The present inventors have found that somatic mutations in the GPAM gene that lead to impaired or abrogate function of the GPAM protein are particularly useful markers for the diagnosis and/or prognosis of NAFLD or ARLD.

Accordingly, in certain embodiments, step b) of the method of the invention comprises detecting one or more somatic mutations in the GPAM gene that confer a selective advantage on a liver cell of the subject. In certain preferred embodiments, the one or more somatic mutations detected in the GPAM gene impair or abrogate the function of the GPAM protein. Examples of somatic mutations in the GPAM gene that may be detected in step b) of the method of the invention include mutations resulting in G790W, E730K, A619P, R519G, H486R, L332S, F313L, Y292C, G273V, L225M and/or Q210R substitutions of the GPAM protein, a L322 frameshift mutation or a R118 (i.e. R118*) nonsense mutation of the GPAM protein.

The present inventors have also found that somatic mutations within the CIDEB gene may be used as an indicator of NAFLD or ARLD in a subject, an indicator of an increased risk of the subject developing NAFLD or ARLD, and/or a further indicator of an increased risk of the subject developing a more severe form of liver disease or an associated disease or condition. CIDEB is the major member in the CIDE family active in hepatocytes, and knock-out mouse models show resistance to dietary steatohepatitis and increased insulin sensitivity (Li et al., Diabetes, 2007, 56, 2523-2532). The CIDE proteins regulate fusion of intracellular lipid droplets, mediated by the formation of homodimers between CIDE proteins on different droplets (Barneda et al. Elife, 2015, 4, 1-24, and Sun et al. Nat. Commun. 2013, 4, 15949). Homodimerisation of CIDE proteins occurs through electrostatic contacts between positively charged residues on the CIDE protein from one lipid droplet and negatively charged residues on the other.

The present inventors have identified nonsense, stop-loss mutation and missense mutations in the CIDEB gene of patients suffering from NAFLD or ARLD. In particular, the present inventors found that the missense mutations were predominantly located in the two domains implicated in homodimerisation of CIDE proteins, and that many of them either switched a charged residue for a neutral one (R45W, R45Q, K140N, R144P) or reversed the charge (D42H, K62E, E78K). Previous in vitro mutagenesis studies have shown that mutations causing substitutions at charged residues or truncation of the CIDEB protein disrupt growth of lipid droplets in liver cells (Barneda et al. Elife, 2015, 4, 1-24, and Sun et al. Nat. Commun. 2013, 4,1594). CIDEB knock-out mice have also been found to be resistant to steatohepatitis caused by high-fat diets (Li et al. Diabetes, 2007, 56, 2523-2532), thus providing in vivo evidence of how inactivating CIDEB mutations might confer selective advantage on hepatocytes in metabolic liver diseases.

Accordingly, in certain embodiments, step b) of the method of the invention comprises detecting one or more somatic mutations within the CIDEB gene that confer a selective advantage on a liver cell of the subject. In certain preferred embodiments, the one or more somatic mutations detected in the CIDEB gene impairs or abrogates the function of a CIDEB protein expressed by a liver cell of the subject. For example, the somatic mutation present in the CIDEB gene may be one which results in the alteration in the charge of a CIDEB protein, for example by the substitution of a charged residue in a CIDEB protein with a neutral residue, the substitution of a neutral residue in a CIDEB protein with a charged residue, the substitution of negatively charged residue in a CIDEB protein with a positively charged residue, and/or the substitution of a positively charged residue in a CIDEB protein with a negativity charged residue. In exemplary embodiments, the one or somatic mutations in the CIDEB gene impairs or abrogates homodimerisation of a CIDEB protein, thus preventing fusion and growth of lipid droplets within the liver cell. Examples of somatic mutations in the CIDEB gene that may be detected in step b) of the method of the invention include mutations resulting in L4H, L7Q, F23S, W28R, D42H, R45Q, R45W, K62E, E72K, R122Q, I131V, A123P, K140N, R144P, L150Q, N151D, D165E and/or Q167E substitutions of the CIDEB protein, or W28 (i.e. W28*), W181 (i.e. W181*) and/or 220Y (i.e. *220Y) nonsense mutations of the CIDEB protein.

The present inventors have also found that the detection of somatic mutations within the ACVR2A gene may be used as an indicator of NAFLD or ARLD in a subject, an indicator of an increased risk of the subject developing NAFLD or ARLD, and/or an indicator of an increased risk of the subject developing a more severe form of liver disease or an associated disease or condition. The ACVR2A gene encodes a receptor for Activin-A in the TGF-β superfamily. The present inventors have identified 13 missense mutations, 2 nonsense and 1 splice-site indel in ACVR2A (q=7×10⁻⁹), as well as 4 large-scale deletions through structural variation, in patients suffering from NAFLD or ARLD.

Accordingly, in certain embodiments, step b) of the method of the invention comprises detecting one or more somatic mutations within the ACVR2A gene that confer a selective advantage on a liver cell of the subject.

Examples of somatic mutations in the ACVR2A gene that may be detected in step b) of the method of the invention include mutations resulting in a C30R, L31P, C85Y, C105S, Y236C, M241V, V283G, T295A, H320Y, I375V, S424Y and Q481P substitution of the ACVR2A protein, and a R41 (i.e. R41*) or E234 (i.e. E234*) nonsense mutation of the ACVR2A protein.

The present inventors have also found that the detection of somatic mutations within the ALB gene may be used as an indicator of NAFLD or ARLD in a subject, an indicator of an increased risk of the subject developing NAFLD or ARLD, and/or an indicator of an increased risk of the subject developing a more severe form of liver disease or an associated disease or condition. The ALB gene encodes for serum albumin, which is synthesized in the liver as preproalbumin. The nascent form of preproalbumin is processed and released into circulation as serum albumin.

Accordingly, in certain embodiments, step b) of the method of the invention comprises detecting one or more somatic mutations within the ALB gene that confer a selective advantage on a liver cell of the subject. In certain preferred embodiments, somatic mutation within the ALB gene may be detected at the protein level by, for example, characterising serum albumin present in a blood sample obtained from a subject. For example, the presence and/or level of mutated serum albumin in a blood sample may be determine by, for example, enzyme-linked immunosorbent assay (ELISA), Western Blot analysis and mass spectrometry.

The present inventors have also found that somatic mutations within the TNRC6B gene may be used as further indicator of NAFLD or ARLD in a subject, a further indicator of an increased risk of the subject developing NAFLD or ARLD and/or a further indicator of an increased risk of the subject developing a more severe form of liver disease or an associated disease or condition. TNRC6B encodes a protein involved in microRNA processing (Meister et al., Curr Biol. 2005, 6;15(23):2149-55). The present inventors have identified somatic mutations in the TNRC6B gene in patients suffering from NAFLD or ARLD. In particular, the present inventors found 3 nonsense, 2 essential splice site and one large in-frame deletion, as well as 3 missense mutations in the TNRC6B gene of patients suffering from NAFLD or ARLD.

Accordingly, in certain embodiments, step b) of the method of the invention comprises detecting one or more somatic mutations in the TNRC6B gene that confer a selective advantage on a liver cell of the subject. In certain preferred embodiments, the one or more somatic mutations in the TNRC6B gene impair or abrogate the function of a TNRC6B protein.

Examples of somatic mutations in the TNRC6B gene that may be detected in step b) of the method of the invention include mutations resulting in G1374C, T1535S and/or M1814V substitutions of the TNRC6B protein, G536 (i.e. G536*), W1399 (i.e. W1399*) or 01700 (i.e. 01700*) nonsense mutations of the TNRC6B protein, and/or an in-frame deletion of residues 163-180 of the TNRC6B protein.

In certain exemplary embodiments, step b) of the method of the invention comprises detecting one or more somatic mutations in the FOXO1, GPAM and/or CIDEB genes. That is to say, that at least one somatic mutation is detected in the FOXO1 gene, at least one somatic mutation is detected in the GPAM gene, and/or at least one somatic mutation is detected in the CIDEB gene. For example, the method may comprise the detection of one or more somatic mutations in the FOXO1 gene in the region of DNA of the FOXO1 gene that encodes the 14-3-3 protein binding motif of the FOXO1 protein, the detection of one or more mutations in the GPAM gene that encodes a GPAM protein that displays impaired or abrogated function, and/or the detection of one or more mutations in the CIDEB gene that encodes a CIDEB protein that displays impaired or abrogated function.

The step of detecting somatic mutations that confer a selective advantage on one or more liver cells of the subject (for example, the detection of one or more somatic mutations in a gene selected from the group consisting of FOXO1, GPAM, CIDEB, ACVR2A, ALB and TNRC6B) may be achieved by sequencing DNA obtained from a biological sample obtained from a subject, as described herein. Additionally or alternatively, the one or more somatic mutations may be detected by sequencing and/or quantifying mRNA in the biological sample that is transcribed from a gene comprising one or more somatic mutations. For example, the one or more somatic mutations may be detected by sequencing and/or quantifying mRNA in the biological sample that is transcribed from a FOXO1, GPAM, CIDEB, ACVR2A, ALB or TNRC6B gene comprising one or more somatic mutations.

A variety of DNA and RNA sequencing procedures are known in the art and may be used to practice the methods disclosed herein. For example, Sanger sequencing, Polony sequencing, 454 pyrosequencing, Combinatorial probe anchor synthesis, SOLiD sequencing, Ion Torrent semiconductor sequencing, DNA nanoball sequencing, Heliscope single molecule sequencing, Single molecule real time (SMRT) sequencing, Nanopore DNA sequencing, Microfluidic Sanger sequencing and Illumina dye sequencing.

Additionally or alternatively, the one or more somatic mutations may be detected and/or quantified by detecting a protein (for example, a FOXO1, GPAM, CIDEB, ACVR2A, ALB or TNRC6B protein) comprising a mutated amino add encoded by a gene comprising one or more somatic mutations. The presence and/or level of mutated protein in a sample may be measured using any suitable technique known in the art. The method may comprise isolating the protein from the sample and then assessing the quantity of the mutated protein present. Depending on the method used and the level of accuracy required, a purification step may be carried out. In some circumstances, simple lysis of cells in the sample may be sufficient. The level of mutated protein present may be assessed using one or more techniques selected from enzyme-linked immunosorbent assay (ELISA), Western Blot analysis and mass spectrometry.

For the avoidance of doubt, the term “severe form of liver disease” as used herein refers to progressive liver fibrosis or liver-related dysfunction as a consequence of NAFLD or ARLD, for example, non-alcoholic steatohepatitis (NASH), liver fibrosis, cirrhosis, liver cancer, and liver failure. Specific types of liver cancer include hepatocellular carcinoma (HCC), intrahepatic cholangiocarcinoma, angiosarcoma and hemangiosarcoma.

For the avoidance of doubt, the term “associated disease or condition” as used herein refers to diseases and conditions that are associated with liver disease. Such diseases and conditions include gastrointestinal cancers of the oesophagus, stomach, bile ducts, gallbladder and associated structures, pancreas, small intestine, large intestine, rectum and anus. Other examples of diseases and conditions associated with liver disease include obesity, type 2 diabetes mellitus, hypertension, dyslipidaemia (e.g. hypercholesterolaemia) and cardiovascular disease. Thus, in certain embodiments, step b) of the method of the invention comprises determining if the subject is suffering from NAFLD or ARLD, is at risk of developing NAFLD or ARLD, and/or is at risk of developing a more severe form of liver disease selected from the group consisting of non-alcoholic steatohepatitis (NASH), liver fibrosis, liver cirrhosis, cancer (for example hepatocellular carcinoma (HCC)) and liver failure. In certain embodiments, step b) of the method of the invention comprises determining if the subject is at risk of developing a disease or condition associated with liver disease selected from the group consisting of gastrointestinal cancer, obesity, type 2 diabetes mellitus, hypertension, dyslipidaemia (e.g. hypercholesterolaemia) and cardiovascular disease.

In certain embodiments, step b) of the method of the invention comprises quantifying the level of one or more somatic mutations in the biological sample provided in step a). Techniques suitable for quantifying the level of one or more somatic mutations are known in the art, and include, for example, the use of qPCR, RT-qPCR, DNA/RNA microarrays and analysis of DNA sequencing data, particularly next-generation sequencing (e.g. Illumina dye sequencing and Ion Torrent semiconductor sequencing) data.

As used herein, the terms “level of one or more somatic mutations” and “level of a somatic mutation” refer to the copy number of one or more somatic mutations in a given biological sample, the concentration of DNA molecules comprising the one or more somatic mutations in a given biological sample, the concentration of mRNA molecules comprising the one or more somatic mutations in a given biological sample, or the concentration of a protein comprising the one or more mutated amino acid encoded by the one or more somatic mutations in a given biological sample.

Typically, the level (i.e. as assessed by the copy number of a mutation, the concentration of DNA with the mutation, the concentration of mRNA with the mutation and/or the concentration of protein with the mutation) of one or more somatic mutations in a given biological sample is compared to the level of the one or more somatic mutations in a reference biological sample obtained from a healthy subject (i.e. a liver sample obtained from a subject that does not have NAFLD or ARLD), a reference biological sample obtained from a subject known to have NAFLD or ARLD, and/or a reference biological sample obtained from the same subject.

In embodiments wherein the reference biological sample is from a healthy subject, if the level of one or more somatic mutations in the given biological sample from the subject is higher than the level in the reference biological sample, this indicates that the subject has NAFLD or ARLD, has an increased risk of developing NAFLD or ARLD, has an increased risk of developing a more severe form of liver disease and/or has an increased risk of developing an associated disease or condition. If the level of each one or more somatic mutations in a given biological sample from the subject is comparable to, or lower than, the level in the reference biological sample obtained from a healthy subject this indicates that the subject does not have NAFLD or ARLD, does not have an increased risk of developing NAFLD or ARLD, does not have an increased risk of developing a more severe form of liver disease and/or does not have an increased risk of developing a disease or condition associated with liver disease.

In embodiments wherein the reference biological sample is from a subject known to have NAFLD or ARLD, if the level of one or more somatic mutations in the given biological sample from the subject is comparable to, or higher than, the level in the reference biological sample, this indicates that the subject has NAFLD or ARLD, has an increased risk of developing NAFLD or ARLD, has an increased risk of developing a more severe form of liver disease and/or an increased risk of developing an associated disease or condition. If the level of each one or more somatic mutations in the given biological sample from the subject is lower than the level in the reference biological sample this may indicate that the subject does not have NAFLD or ARLD, does not have an increased risk of developing NAFLD or ARLD, does not have an increased risk of developing a more severe form of liver disease and/or does not have an increased risk of developing an associated disease or condition.

In certain embodiments the reference biological sample is from the same subject as the given biological sample. Typically, the reference biological sample from the same subject is obtained from a different region of the body or liver of the subject, and/or obtained from the subject at a different time point to the given biological sample. For example, the reference biological sample may be obtained from the subject, 1 to 6 months earlier, 6 to 12 months earlier, 1 to 2 years earlier or 2 to 3 years earlier than the given biological sample obtained from the subject.

In embodiments wherein the reference biological sample is obtained from the same subject but at an earlier time point to the given biological sample, if the level of one or more somatic mutations in the given biological sample is higher than the level in the reference biological sample, this may indicate that the subject has an increased risk of developing NAFLD or ARLD, has an increased risk of developing a more severe form of liver disease and/or has an increased risk of developing an associated disease or condition compared to the earlier time point when the reference biological sample was obtained from the subject. Alternatively, if the level of one or more somatic mutations in the given biological sample is higher than the level in the reference biological sample, this may indicate that the subject has developed NAFLD or ARLD, has developed a more severe form of liver disease and/or has developed an associated disease or condition.

If the level of each one or more somatic mutations in the given biological sample from the subject is comparable to the level in the reference biological sample this may indicate that the subject has not developed NAFLD or ARLD, has not developed a more severe form of NAFLD or ARLD, has not developed a more severe form of liver disease and/or has not developed an associated disease or condition. If the level of each one or more somatic mutations in the given biological sample from the subject is lower than the level in the reference biological sample this may indicate that the subject has a decreased risk of developing NAFLD or ARLD, has a decreased risk of developing a more severe form of liver disease and/or has a decreased risk of developing an associated disease or condition compared to the earlier time point when the reference biological sample was obtained from the subject. Or, if the level of each one or more somatic mutations in the given biological sample from the subject is lower than the level in the reference biological sample this may indicate that the subject has NAFLD or ARLD that is in remission, has a more severe form of liver disease that is in remission and/or has a an associated disease or condition that is in remission.

In certain embodiments, step b) of the method of the invention may comprise detecting and/or quantifying one or more somatic mutations derived from different liver cell clones contained in a given biological sample obtained from a subject. As used herein, the term “liver cell clone” refers to a group of identical liver cells that share a common ancestry. In certain embodiments, the detection of the one or more somatic mutations in step b) of the method of the invention (for example, one or more somatic mutations in a gene selected from the group consisting of FOXO1, GPAM, CIDEB, ACVR2A, ALB and TNRC6B) may comprise detecting and/or quantifying one or more somatic mutations derived from different liver cell clones contained in different biological samples obtained from the subject. For example, the different liver cell clones may be contained in different biological samples obtained from different regions of the liver and/or different biological samples obtained from the subject at different time points. The detection of one or more of the same somatic mutations in one or more (for example, 2, 3, 4, 5, 6 or more) different liver cell clones that confer a selective advantage on the liver cells of the respective liver cell clone is a particularly strong indicator of the presence of NAFLD or ARLD in a subject. Thus, in certain embodiments, step b) of the method of the invention comprises detecting and/or quantifying one or more somatic mutations in the DNA, RNA and/or protein derived from different liver cell clones contained in the biological sample obtained from the subject, wherein the presence of the same somatic mutation that confers a selective advantage on the liver cells of said liver cell clone in more than one liver cell clone indicates that the subject is suffering from NAFLD or ARLD, is at risk of developing NAFLD or ARLD, is at risk of developing a more severe form of liver disease, and/or is at risk of developing a disease or condition associated with liver disease.

The detection and/or quantification of the level of one or more somatic mutations that confer a selective advantage on liver cells, and/or liver cell clones, obtained from a subject can accordingly be used to guide a clinician in determining a suitable disease monitoring program, suitable treatment or to inform the clinician that the subject is in remission.

In embodiments wherein the biological sample comprises DNA, RNA and/or protein from a liver biopsy sample, the size and/or proportion of a liver biopsy sample, or microdissection thereof, that comprises liver cells having one or more somatic mutations that confer a selective advantage on the liver cells may be measured. The size and/or proportion of the liver biopsy sample, or microdissection thereof, that comprises liver cells having on or more somatic mutations that confer a selective advantage on the liver cells provides further insight into the severity and/or progression of NAFLD or ARLD in a subject. For example, a liver biopsy sample having a size of 1 cm³ which comprises ≥0.5 cm³ (i.e. a proportion of 50% or more of the liver biopsy sample) of liver cells having one or more somatic mutations that confer a selective advantage on the liver cells, may be considered to indicate that the subject has NAFLD or AFLD in a significant proportion of their liver (for example, the subject may have steatosis in at least 5%, 10%, 20%, 30%, 40%, 50%, or more, of their liver). Thus, in certain embodiments, the method of the invention further comprises a step of measuring the size and/or proportion of the liver biopsy sample, or microdissection thereof, that comprises liver cells having one or more somatic mutations that confer a selective advantage on the liver cells. The size and/or proportion of the liver biopsy sample, or microdissection sample thereof, that comprises liver cells having one or more somatic mutations that confer a selective advantage on the liver cells can accordingly be used to guide the clinician in determining a suitable disease monitoring program, suitable treatment or to inform the clinician that the subject is in remission.

For the avoidance of doubt, step b) of the method of the invention may include the detection and/or quantification of a somatic mutation in any one or more of the following:
a DNA (e.g. cfDNA) molecule comprising a gene;
a DNA (e.g. cfDNA) molecule comprising a portion of a gene;
an RNA molecule comprising a sequence corresponding to a portion of a gene;
a protein encoded by a sequence corresponding to a gene; and/or
a protein encoded by a sequence corresponding to a portion of a gene.

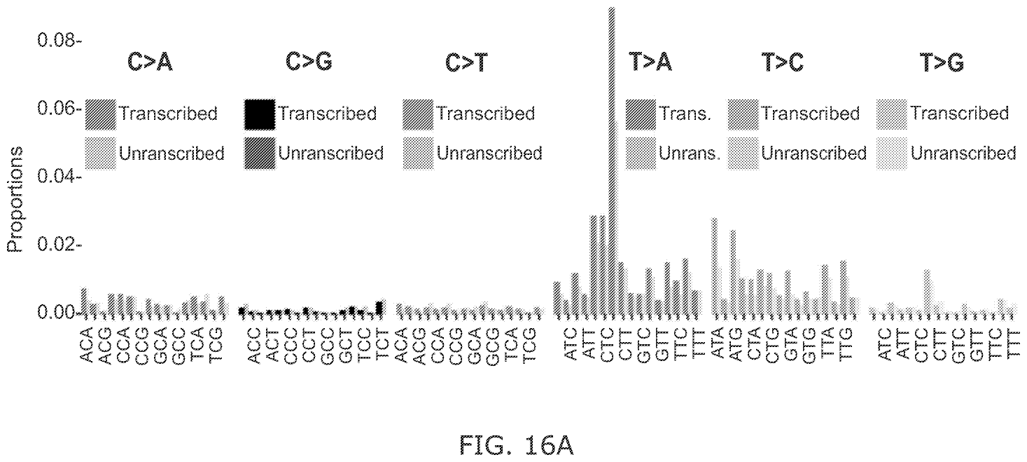

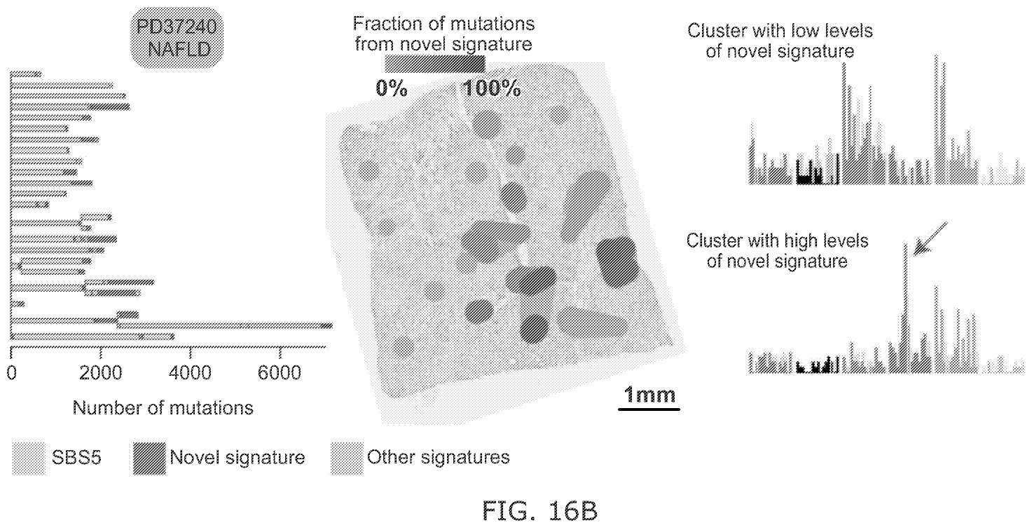

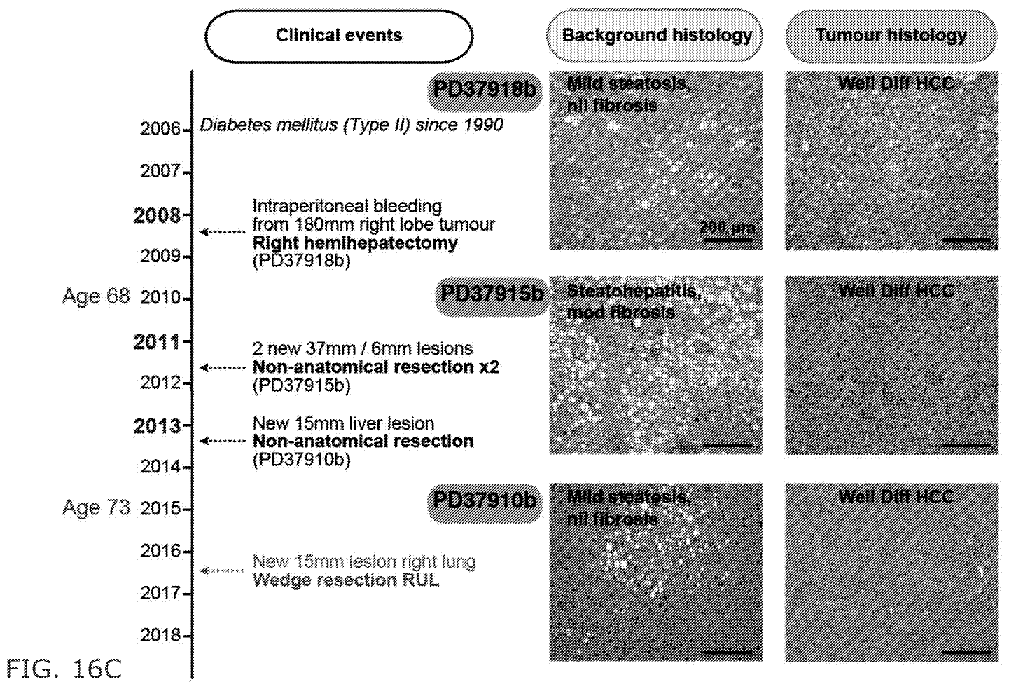

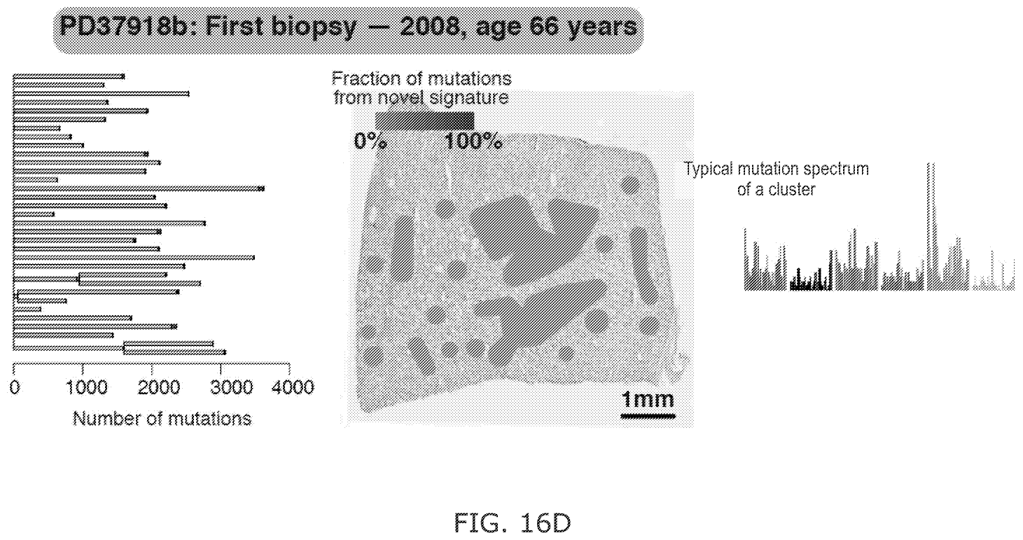

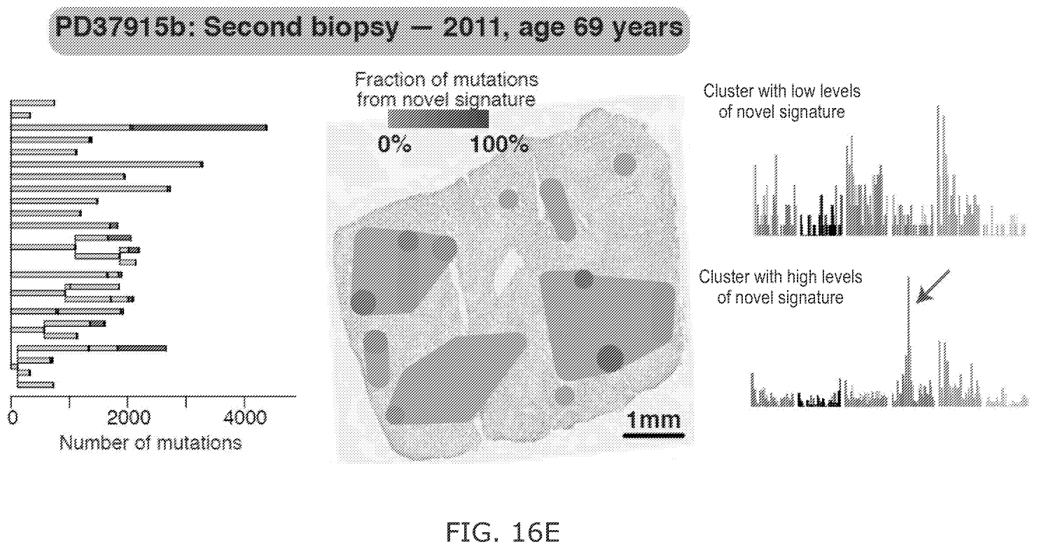

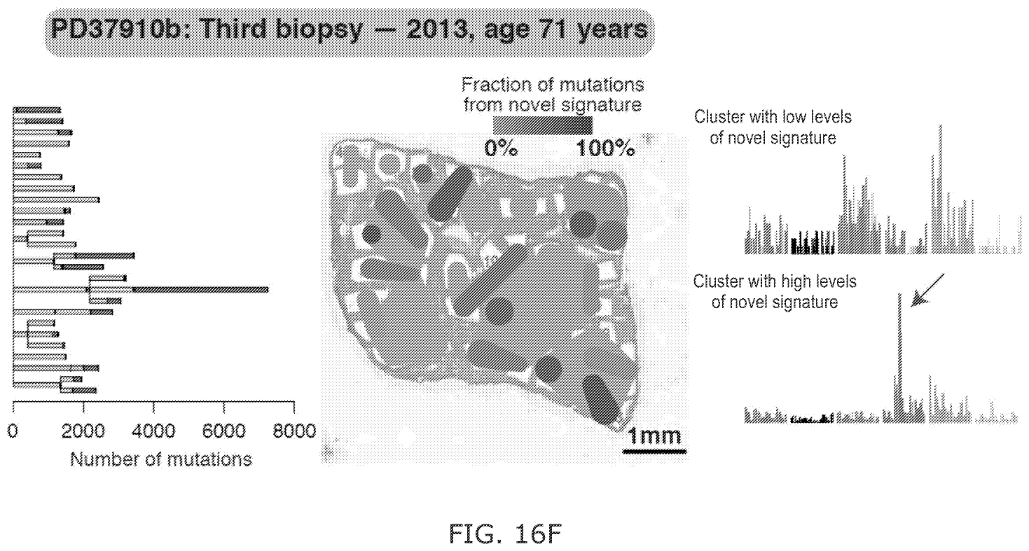

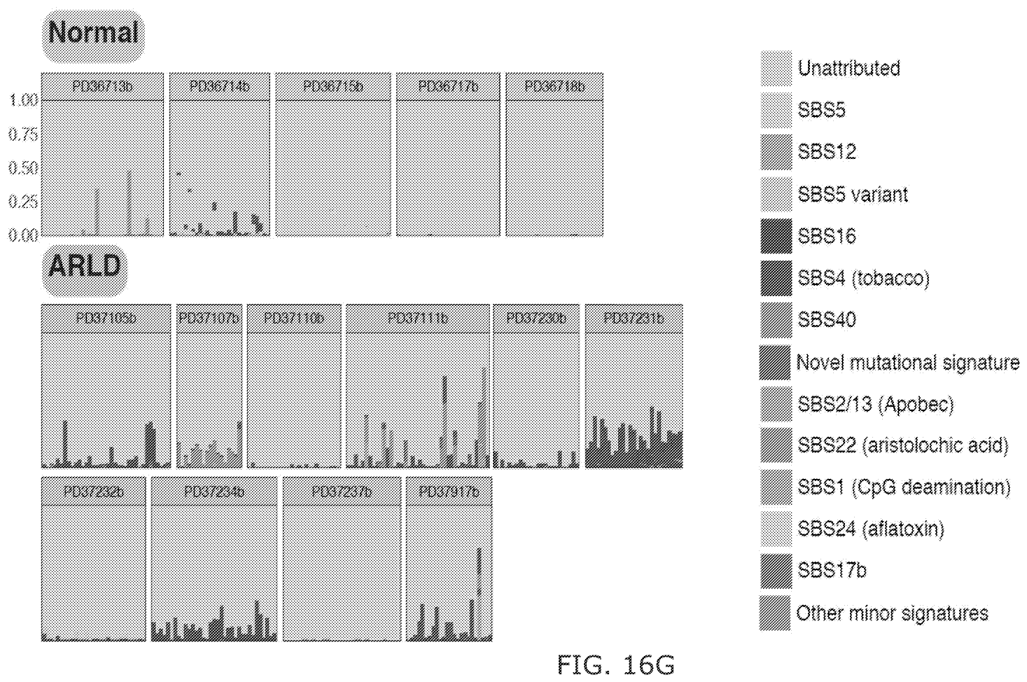

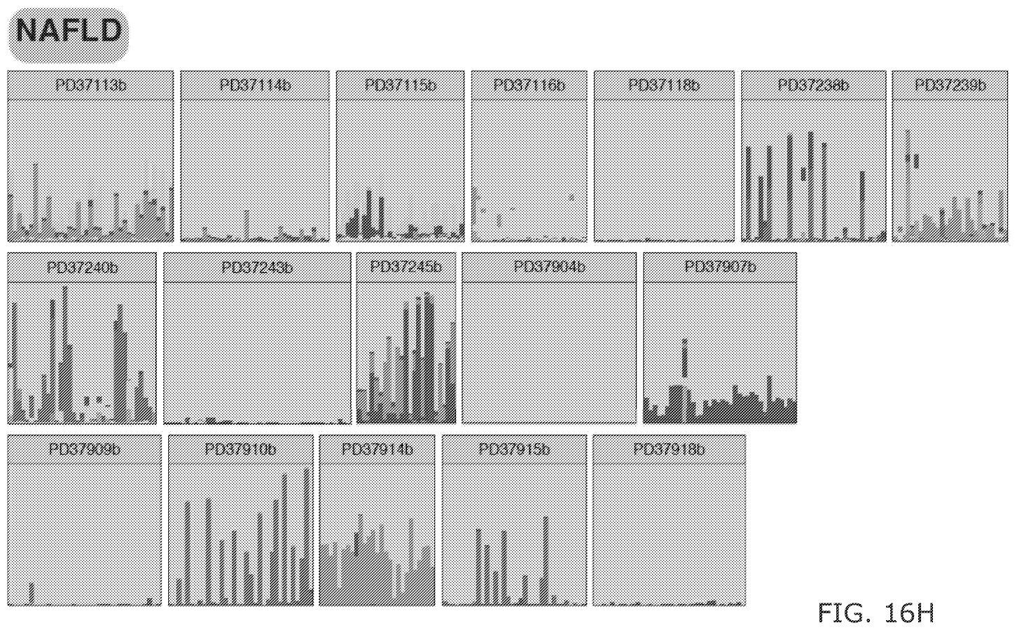

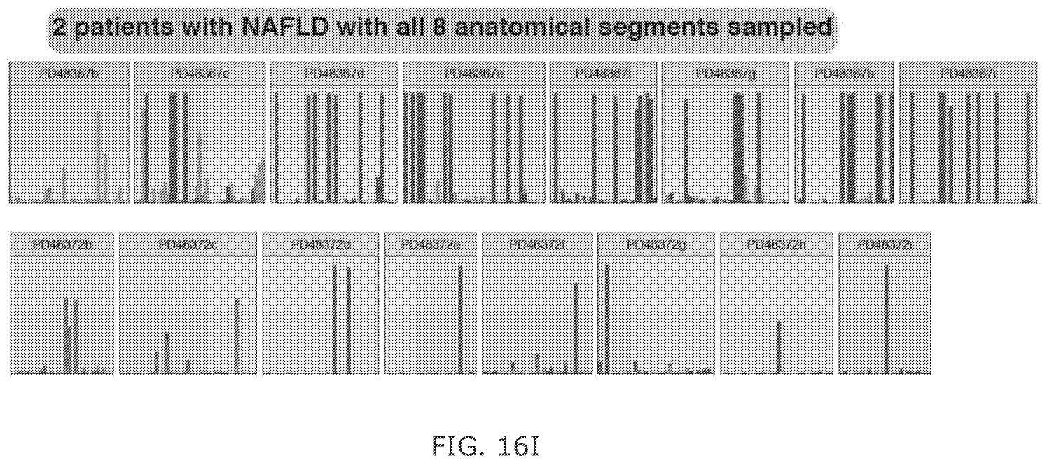

The method of the invention may additionally comprise the detection and/or quantification of one or more additional markers to improve the confidence of the diagnosis or prognosis of NAFLD or ARLD in the subject. For example, as described herein, the present inventors have noted a new mutational signature in the liver cells of subjects with NAFLD or ARLD. In particular, the present inventors noted that the new mutational signature increases in intensity as the liver disease progresses (see FIG. 16D-F). Thus, in certain embodiments, the method of the invention may further comprise the identification, and optional monitoring, of mutational signatures in DNA, RNA and/or protein derived from one or more liver cells of a subject.

For the avoidance of doubt, as used herein, the term “mutational signature” means a set of somatic mutations that is observed in a subject known or suspected of having NAFLD or ARLD. Methods for identifying s mutational signatures in a cell, such as liver cell, are known in the art. An exemplary method is described herein which involves the use of the SigProfilerMatrixGenerator software (Bergstrom, E. N. et al. BMC Genomics 20,1-12 (2019)). In certain embodiments, the method of the invention may also comprise a step of detecting one or more somatic mutations within the CLCN5 gene in the biological sample obtained from the subject. The present inventors have found that somatic mutations in the CLCN5 gene occur in patients suffering from NAFLD or ARLD. The CLCN5 gene encodes a chloride channel, which causes X-linked nephrocalcinosis but no known liver phenotype when mutated in the germline. Somatic mutations within the CLCN5 gene may be a further indicator of NAFLD or ARLD in a subject, a further indicator of an increased risk of the subject developing NAFLD or ARLD, and/or a further indicator of an increased risk of the subject developing a more severe form of liver disease or an associated disease or condition. In certain exemplary embodiments, the one or more somatic mutations detected in the CLCN5 gene is one that confers a selective advantage on the liver cell(s) containing the mutation.

Examples of somatic mutations in the CLCN5 gene that may be detected in the method of the invention include mutations resulting in G330D, 5371G and/or G570R substitutions of the CLCN5 protein, or C171 (i.e. C171*), W192 (i.e. W192*), E676 (i.e. E676*) or G791 (i.e. G791*) nonsense mutations of the CLCN5 protein.

In certain embodiments, the method of the invention may also comprise a step of detecting one or more non-coding somatic mutations in the biological sample obtained from the subject. The present inventors have detected non-coding mutations in the NEAT1 gene of patients suffering from NAFLD or ARLD. Thus, in certain embodiments, the method of the invention further comprises detecting one or more non-coding somatic mutations in the NEAT1 gene. In certain embodiments, the one or more non-coding somatic mutations detected in the NEAT1 gene is one that confers a selective advantage on the liver cell(s) containing the mutation.

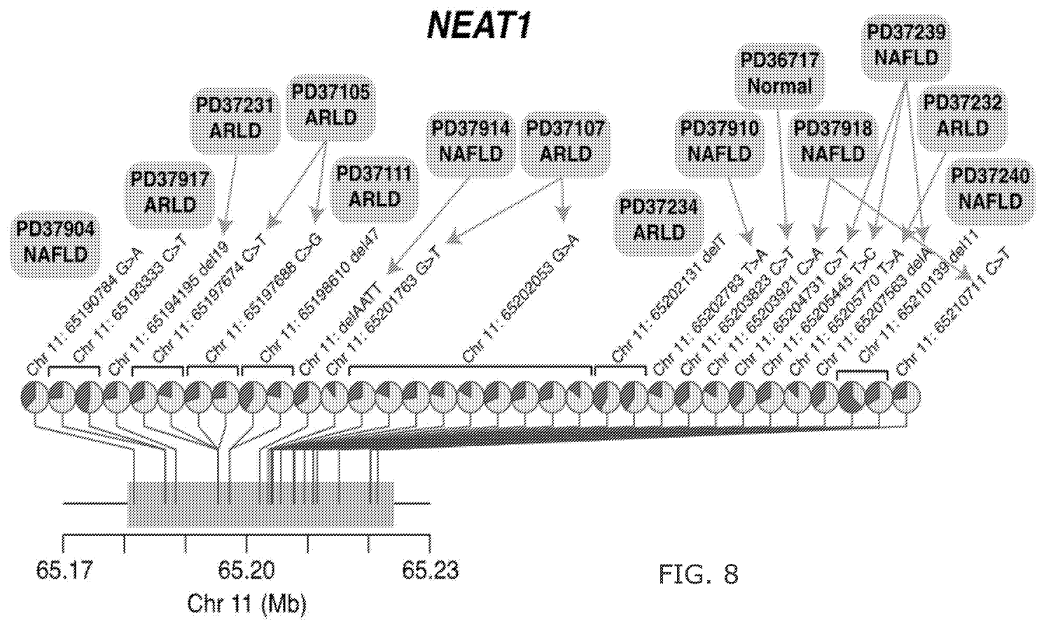

Examples of non-coding somatic mutations in the NEAT1 gene that may be detected in the method of the invention include the following mutations on human chromosome 11 which are provided by reference to their genomic location according to the GRCh37d5 human reference genome: 65190784 G>A; 65193333 C>T; 65194195 del19; 65197674 C>T; 65197688 C>G; 65198610 del47; 65201066 del AATT (referred to as “delAATT” in FIG. 8); 65/201,763 G>T; 65202053 G>A; 65202131 delT; 65/202,783 T>A; 65203823 C>T; 65203921 C>A; 65204731 C>T; 65205445 T>C; 65205770 T>A; 65207563 delA; 65/210,139 del11; and 65/210,711 C>T (also see FIG. 8).

In certain embodiments, the method of the invention may also comprise a step of determining the telomere length of a DNA molecule in the biological sample; and comparing the telomere length of the DNA molecule in the biological sample with the telomere length of a DNA molecule obtained from a normal liver cell. Previous reports have suggested that chronic liver diseases, such as NAFLD or ARLD are associated with telomere shortening (Wiemann et al., FASEB J, 2002, 16, 935-42). That is to say that, in diseased liver cells, telomere length is often shorter than telomere length in non-diseased liver cells. Thus, the telomere length of a DNA molecule in a biological sample obtained from a subject may be used as a further indicator of NAFLD or ARLD in a subject, a further indicator of an increased risk of the subject developing NAFLD or AFLD, a further indicator of an increased risk of the subject developing a more severe form of liver disease and/or a further indicator of an increased risk of the subject developing an associated disease or condition. Thus, in certain embodiments, the method of the invention may further comprise the steps of
c) determining the telomere length of a DNA molecule in the biological sample; and
d) comparing the telomere length of the DNA molecule in the biological sample with the average telomere length of a DNA molecule obtained from a normal liver cell (e.g. a liver cell obtained from a subject who is not suffering from NAFLD or ARLD, or from a region of the subject's liver that is healthy). Typically, the DNA molecule is obtained from a normal liver cell that is from the same subject or from a normal liver cell from an age-matched subject who is not suffering from NAFLD or ARLD. In certain exemplary embodiments, the method may further comprise the steps of
c) determining the average telomere length of the DNA molecules in the biological sample; and
d) comparing the average telomere length of the DNA molecules in the biological sample with the average telomere length of DNA molecules obtained from a normal liver cell. As used herein the term “average telomere length” is the average length of the telomeres measured in a given sample. The average telomere length may be the mean, median or mode telomere length for a given sample. Typically, the average telomere length is the mean telomere length for a given sample. Alternatively, the average telomere length may be the mean, median or mode telomere length for clone in a given sample.

Additional risk factors may also be taken into account when diagnosing or prognosing NAFLD or ARLD in a subject. Additional risk factors typically include: age, the presence of metabolic syndrome, gender, ethnic group, dietary factors, smoking status (e.g. current smoker or former smoker), obstructive sleep apnea, obesity, type 2 diabetes mellitus, hypertension, dyslipidaemia (e.g. hypercholesterolaemia) and cardiovascular disease. Genetic factors in addition to somatic mutations (for example, somatic mutations within the FOXO1, GPAM, CIDEB, ACVR2A, ALB and TNRC6B, and also the CLCN5 and NEAT1 genes) may also be taken into account in the methods of the invention. For example, germline polymorphisms in the Patatin-like phospholipase domain-containing 3 (PNPLA3) gene are known to increase genetic risk to NAFLD (Sookoian et al. J Lipid Res 2009; 50:2111-6), and may therefore be taken into account. As a further example, germline polymorphisms in genes associated with insulin resistance or lipid synthesis, such as germline polymorphisms in FOXO1 and GPAM, may also be taken into account. Examples of germline polymorphisms suspected of being associated with NAFLD or ARLD are described in Valenti et al., Journal of Heptaology, 2009, 50, 5265 and Hakim et al, Hepatology, 2021, doi: 10.1002/hep.32038.

In exemplary embodiments, the method of the invention may further comprise step c), which comprises calculating a score based on the presence and/or level (for example, copy number) of one or more somatic mutations that confers a selected advantage on one or more liver cells of the subject, and additionally based on one or more further genetic and/or physical characteristics of the liver cells of the subject, such as one or more of the following characteristics:
the presence and/or development of a mutational signature in the liver cells of a subject;
the presence and/or level (for example, copy number) of somatic mutations in the CLCN5 gene;
the presence and/or level (for example, copy number) of non-coding somatic mutations, for example non-coding somatic mutations in the NEAT1 gene;
the size and/or proportion of the liver biopsy sample from which the DNA, RNA and/or protein is derived, that harbours one or more somatic mutations that confer a selective advantage on the liver cells in the liver biopsy sample;
the telomere length of the DNA derived from the one or more liver cells from the subject; and/or
the additional risk factors of the subject, such as age, the presence of metabolic syndrome, gender, ethnic group, dietary factors, smoking status (e.g. current smoker or former smoker), obstructive sleep apnea, obesity, type 2 diabetes mellitus, hypertension, dyslipidaemia (e.g. hypercholesterolaemia) and cardiovascular disease.

The calculation of a score based on the presence and/or level (for example, copy number) of a somatic mutation in the one or more liver cells of the subject, and one or more of the above-mentioned genetic and/or physical characteristics may provide further guidance to a clinician in determining a suitable disease monitoring program, suitable treatment or to inform the clinician that the subject is in remission.

Methods of Monitoring NALFLD or ARLD Progression in a Subject:

The present invention also provides a method for identifying a subject suffering from NAFLD or ARLD who would benefit from increased disease monitoring, wherein said method comprises steps a) and b) described hereinabove. Increased monitoring of the subject may involve monitoring the subject at regular intervals (for example once every year, once every 6 months or once every 3 months) using monitoring techniques suitable for NAFLD or ARLD such as computerized tomography (CT) scanning and/or blood testing to monitor circulating liver enzyme levels.

The diagnostic and prognostic methods described herein may also be used to monitor the development or progression of NAFLD or ARLD in a subject. For example, monitoring of NAFLD or ARLD in a subject may be achieved by repeating steps a) and b) of the methods described herein at intervals of days, weeks, months or years. The diagnostic and prognostic methods described herein are particularly suitable for monitoring a subject at risk of developing NAFLD or ARLD, monitoring a subject at risk of developing a more severe form of liver diseases, monitoring a subject at risk of developing a disease or condition associated with liver disease and/or monitoring NAFLD or ARLD in a subject that is undergoing treatment for NAFLD or ARLD. In exemplary embodiments, steps a) and b) of the diagnostic or prognostic method described herein are carried out once every week, once every two weeks, once every three weeks, once every four weeks, once every month, once every two months, once every three months or once every six months. In certain exemplary embodiments, steps a) and b) are repeated at intervals of once every two years, once every year, two times every year or three times every year. For example, steps a) and b) may be repeated once every two years, once every year, two times every year or three times every year fora period of 1 to 5, 1 to 10 years, 1 to 20 years, 1 to 30 years or 1 to 40 years.

In Vivo Methods for Diagnosing or Prognostication of NALFLD or ARLD:

The present invention also provides a method for diagnosing or prognostication of NAFLD or ARLD in a subject, said method comprising the steps of
a) administering a dose of a diagnostic probe to the subject; and
b) detecting the diagnostic probe in the subject, wherein said diagnostic probe indicates the presence and/or absence of a somatic mutation in a gene selected from the group consisting of FOXO1, GPAM, CIDEB, ACVR2A, ALB and TNRC6B in the liver of the subject, and wherein the presence of a somatic mutation in the FOXO1, GPAM, CIDEB, ACVR2A, ALB or TNRC6B gene indicates that the subject is suffering from NAFLD or AFLD, is at risk of developing NAFLD or ARLD, is at risk of developing a more severe form of liver disease and/or is at risk of developing a disease or condition associated with liver disease such as gastrointestinal cancer.

The present invention also provides a method for diagnosing or prognostication of NAFLD or ARLD in a subject, wherein
the subject is one to whom a diagnostic probe has been administered, and
said method comprises the step of detecting the diagnostic probe in the subject, the diagnostic probe being one that indicates the presence or absence of one or more somatic mutations in a gene selected from the group consisting of FOXO1, GPAM, CIDEB, ACVR2A, ALB and TNRC6B in the liver of the subject,
wherein the presence of one or more somatic mutations indicates that the subject is suffering from NAFLD or ARLD, is at risk of developing NAFLD or ARLD, is at risk of developing a more severe form of liver disease and/or is at risk of developing a disease or condition associated with liver disease such as gastrointestinal cancer.

The present invention also provides a diagnostic probe for use in diagnosing or prognostication of NALFLD or ARLD in a subject, said use comprising
a) administering a dose of the diagnostic probe to the subject; and
b) detecting the diagnostic probe in the subject, wherein said diagnostic probe indicates the presence and/or absence of one or more somatic mutations in a gene selected from the group consisting of FOXO1, GPAM, CIDEB, ACVR2A, ALB and TNRC6B in the liver of the subject, and wherein the presence of one or more somatic mutations indicates that the subject is suffering from NAFLD or ARLD, is at risk of developing NAFLD or ARLD, is at risk of developing a more severe form of liver disease and/or is at risk of developing a disease or condition associated with liver disease such as gastrointestinal cancer.

Diagnostic probes suitable for use in the in vivo method for diagnosing or prognostication of NALFLD or ARLD described herein include, but are not limited to, small molecules, peptides (including cyclic peptides), proteins, nucleic acids (e.g. DNA and RNA nucleotides including, but not limited to, antisense nucleotide sequences, triple helices, siRNA or miRNA, and nucleotide sequences encoding biologically active proteins, polypeptides or peptides), synthetic or natural inorganic molecules and synthetic or natural organic molecules that specifically bind to a FOXO1, GPAM, CIDEB, ACVR2A, ALB or TNRC6B gene comprising one or more somatic mutation, or specifically bind to FOXO1, GPAM, CIDEB, ACVR2A, ALB or TNRC6B protein encoded by its respective gene comprising one or more somatic mutations. Such diagnostic probes may comprise a “label” which is suitable for imaging and/or diagnosing or prognostication of NALFLD in a subject. Suitable labels include, for example, radioisotopes, radionuclides, isotopes, positron emitters, gamma emitters, fluorescent groups, luminescent groups, chromogenic groups, biotin (in conjunction with, for example, streptavidin complexation) or photoaffinity groups. The type of label chosen will depend on the desired detection method. In certain embodiments, the diagnostic probe for use in diagnosing or prognostication of NAFLD comprises a fluorescent label suitable for fluorescence in situ hybridization (FISH).

For the avoidance of doubt, the in vivo methods described herein may comprise the use of diagnostic probes that enable the detection of one or more somatic mutations in a gene selected from the group consisting of FOXO1, GPAM, CIDEB, ACVR2A, ALB and TNRC6B, and that said at least one or more somatic mutations confer a selective advantage a liver cell of the subject.

Selection of NAFLD or ARLD Patients Suitable for Treatment:

The present invention also provides a method for identifying a subject suffering from NAFLD or ARLD who would benefit from treatment with a therapeutic agent that inhibits or modulates FOXO1, GPAM, CIDEB, ACVR2A, or TNRC6B protein activity. In certain embodiments, the method comprises the steps of
a1) providing a biological sample obtained from the subject; and
b1) detecting one or more somatic mutations in a gene selected from the group consisting of FOXO1, GPAM, CIDEB, ACVR2A, ALB, and TNRC6B in the biological sample, wherein the presence of one or more somatic mutations indicates that the subject is one who would benefit from treatment with a therapeutic agent that inhibits or modulates FOXO1, GPAM, CIDEB, ACVR2A, or TNRC6B protein activity.

In certain embodiments, the present invention concerns a method for identifying a subject suffering from NAFLD or ARLD who would benefit from treatment with a therapeutic agent that inhibits or modulates FOXO1 or GPAM activity. In embodiments, the method comprises the steps of
a1) providing a biological sample obtained from the subject; and
b1) detecting one or more somatic mutations in the FOXO1 and/or GPAM genes in the biological sample, wherein the presence of one or more somatic mutations indicates that the subject is one who would benefit from treatment with a therapeutic agent that inhibits or modulates FOXO1 or GPAM activity.

Suitable therapeutic agents that may be used to inhibit and/or modulate the activity of the FOXO1, GPAM, CIDEB, ACVR2A, or TNRC6B proteins include, but are not limited to, small molecules, peptides (including cyclic peptides), proteins, nucleic acids (e.g. DNA and RNA nucleotides including, but not limited to, antisense nucleotide sequences, triple helices, siRNA or miRNA, and nucleotide sequences encoding biologically active proteins, polypeptides or peptides), synthetic or natural inorganic molecules, synthetic or natural organic molecules, and CRISPR (Le. clustered regularly interspaced short palindromic repeats). For example, the therapeutic agent may be a small molecular organic compound that binds to, and inhibits the activity of the FOXO1, GPAM, CIDEB, ACVR2A, or TNRC6B protein, or for example, the therapeutic agent may be an miRNA or siRNA molecule that knocks down the expression of the FOXO1, GPAM, CIDEB, ACVR2A, or TNRC6B protein that comprises the one or more somatic mutations.

Specific examples of suitable therapeutic agents that inhibit and/or modulate the activity of the FOXO1 protein include 5-amino-7-(cyclohexylamino)-1-ethyl-6-fluoro-4-oxo-1,4-dihydroquinoline-3-carboxylic acid, which may also be referred to as AS1842856 (see EP1650192A1 and Molecular Pharmacology, 2010 78 (5) 961-970, which are incorporated herein by reference), and compounds disclosed in Langlet et al. (Cell, 2017, 171, 824-835 which is incorporated herein by reference) such as 2-[2-(methylamino)pyrimidin-4-yl]-1,5,6,7-tetrahydropyrrolo[3,2-c]pyridin-4-one (i.e. Compound 8 of Langlet et al. 2017).

A further example of a therapeutic agent suitable for use in the present invention is FSG67, which is a small molecule inhibitor of the GPAM protein (Wydysh et al., J Med Chem. 2009, 28; 52(10):3317-27, and Kuhajda et al., Am J Physiol Regul Integr Comp Physiol. 2011; 301(1): R116-30 which are incorporated herein by reference).

The present invention also provides a therapeutic agent for use in the treatment of NAFLD or ARLD, wherein said use comprises the steps of:
a2) providing a biological sample obtained from the subject;
b2) detecting one or more somatic mutations in a gene selected from the group consisting of FOXO1, GPAM, CIDEB, ACVR2A, ALB and TNRC6B in the biological sample, wherein the presence of one or more somatic mutations indicates that the subject is one who would benefit from treatment with a therapeutic agent that inhibits or modulates FOXO1, GPAM, CIDEB, ACVR2A, ALB or TNRC6B protein activity; and
c2) administering to the subject one or more doses of a therapeutic agent that inhibits or modulates FOXO1, GPAM, CIDEB, ACVR2A, or TNRC6B protein activity.

In certain embodiments, the present invention concerns a therapeutic agent for use in the treatment of NAFLD, wherein said use comprises the steps of:
a2) providing a biological sample obtained from the subject;
b2) detecting one or more somatic mutations in the FOXO1 and/or GPAM genes in the biological sample, wherein the presence of one or more somatic mutations indicates that the subject is one who would benefit from treatment with a therapeutic agent that inhibits or modulates FOXO1 or GPAM protein activity; and
c2) administering to the subject one or more doses of a therapeutic agent that inhibits or modulates FOXO1 or GPAM activity.

For the avoidance of doubt, steps a1), b1), a2) and b2) described herein may independently include any of the features and steps described herein for steps a) and b) of the method for diagnosing or prognostication of NAFLD (or ARLD) according to the present invention described herein above. Also for the avoidance of doubt, step b2) of the methods described herein may comprise detecting one or more somatic mutations in a gene selected from the group consisting of FOXO1, GPAM, CIDEB, ACVR2A, ALB and TNRC6B that confer a selective advantage on one or more liver cells of the subject.

For the avoidance of doubt, it should be understood that the term “a somatic mutation” as used herein includes the term “one or more somatic mutations”. For example, the term “a somatic mutation” may refer to 1, 2, 3, 4, 5, 6, or more, somatic mutations. It should also be understood that any reference herein to “a gene selected from the group consisting of FOXO1, GPAM, CIDEB, ACVR2A, ALB and TNRC6B” includes the selection of one or more of the FOXO1, GPAM, CIDEB, ACVR2A, ALB and TNRC6B genes. That is to say, that the methods of the present invention may comprise, for example, the detection of one or more somatic mutations in the FOXO1 gene, one or more somatic mutations in the GPAM gene, one or more somatic mutations in the CIDEB gene, one or more somatic mutations in the FOXO1 gene, one or more somatic mutations in the ACVR2A gene, one or more somatic mutations in the ALB gene, and/or one or more somatic mutations in the TNRC6B gene.

Kits:

The present invention also provides an in vitro diagnostic kit for use in the diagnosis or prognosis of NAFLD or ARLD (or AFLD) in a subject, said kit comprising one or more reagents for detecting one or more somatic mutations in a gene selected from the groups consisting of FOXO1, GPAM, CIDEB, ACVR2A, ALB and TNRC6B, and optionally comprising one or more reagents for detecting one or more somatic mutations in the CLCN5 gene and/or NEAT1 gene. In certain embodiments, the present invention concerns an in vitro diagnostic kit for use in the diagnosis or prognosis of NAFLD in a subject, said kit comprising one or more reagents for detecting one or more somatic mutations in the FOXO1 and/or GPAM genes, and optionally comprising one or more reagents for detecting one or more somatic mutations in the CLCN5 gene and/or ACVR2A gene.

Optionally, the kit may comprises one or more reagents useful for measuring telomere length.

The term “kit” refers to any item of manufacture (e.g. a package or container) comprising at least one reagent, e.g. a probe or small molecule, for specifically detecting one or more biomarkers of the present invention. The kit may be promoted, distributed, or sold as a unit for performing the methods of the present invention.

The kit may comprises one or more reagents necessary to detect and/or quantify one or more somatic mutations in a gene selected from the groups consisting of FOXO1, GPAM, CIDEB, ACVR2A, ALB and TNRC6B. A kit of the invention may optionally also comprise one or more reagents for detecting and/or quantifying one or more somatic mutations in the CLCN5 gene and/or ACVR2A gene. Particular examples of regents include oligonucleotides that hybridise with a portion of the FOXO1, GPAM, CIDEB, ACVR2A, ALB or TNRC6B gene sequence. The design of such oligonucleotides is within the ability of one skilled in the art. Suitably, such oligonucleotides may be used to amplify, quantify or sequence the FOXO1, GPAM, CIDEB, ACVR2A, ALB or TNRC6B gene sequences comprising one or more somatic mutations.

In certain embodiments, the kit may comprise a binding protein that binds to a FOXO1, GPAM, CIDEB, ACVR2A, ALB or TNRC6B protein comprising one or more mutated amino acids. Suitable binding proteins include polyclonal or monoclonal antibodies that specifically bind to a FOXO1, GPAM, CIDEB, ACVR2A, ALB or TNRC6B protein comprising one or more mutated amino acids. Suitably, such binding proteins may be used in methods for quantifying the level of a FOXO1, GPAM, CIDEB, ACVR2A, ALB or TNRC6B protein comprising one or more mutations in a biological sample.

In certain embodiments, the kit may further comprise one or more reference standards, e.g. a nucleic acid, peptide, polypeptide or protein. Such a reference standard, for example, may be a nucleic acid, peptide, polypeptide or proteins corresponding to the FOXO1, GPAM, CIDEB, ACVR2A, ALB and/or TNRC6B gene or protein that does not comprise one or more somatic mutations, for example, a “wild-type FOXO1” and a “wild-type GPAM” gene or protein, and for example, a wild-type CIDEB, ACVR2A, ALB or TNRC6B protein or gene. The kit may comprise common molecular tags (e.g., green fluorescent protein and beta-galactosidase). Reagents in the kit may be provided in individual containers or as mixtures of two or more reagents in a single container. In addition, instructional materials which describe the use of the components within the kit can be included. The instructional materials may also provide guidance on the interpretation of the results, for example, what level of a particular analyte should be taken to constitute a positive finding.

The kit may also include additional components to facilitate the particular application for which the kit is designed. For example, the kit may additionally contain means of detecting the label (e.g., enzyme substrates for enzymatic labels, filter sets to detect fluorescent labels, appropriate secondary labels such as a sheep anti-mouse-HRP, etc.) and reagents necessary for controls (e.g., control biological samples or standards). A kit may additionally include buffers and other reagents of the necessary grade for use in a method of the disclosed invention in a health care setting. Non-limiting examples include agents to reduce non-specific binding, such as a carrier protein or a detergent.

### EQUIVALENTS

The invention has been described broadly and generically herein. Those of ordinary skill in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

Further, each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

### INCORPORATION BY REFERENCE

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right physically to incorporate into this application any and all materials and information from any such articles, patents, patent applications, or other physical and electronic documents.

The invention will now be illustrated in a non-limiting way by reference to the following Examples.

### EXAMPLES

Material and Methods

Liver Samples:

All biological samples were collected with informed consent from Addenbrooke's Hospital, Cambridge, UK, according to procedures approved by the Local Research Ethics Committee (16/NI/0196). The samples were snap-frozen in liquid nitrogen and stored at −80° C. in the Human Research Tissue Bank of the Cambridge University Hospitals NHS Foundation Trust.

Normal liver tissues were obtained from patients undergoing hepatic resection of colorectal carcinoma metastases; specimens were obtained distant to the metastases and confirmed free of tumour at histopathological examination; one patient (PD36718) had undergone pre-operative portal vein embolization to the ipsilateral liver, but none had received neoadjuvant chemotherapy before resection. Background diseased liver tissue was obtained from subjects with non-alcoholic fatty liver disease (NAFLD) or alcohol-related liver disease (ARLD), undergoing either hepatic resection for hepatocellular carcinoma (HCC) or liver transplantation for HCC or liver failure. None had undergone pre-operative locoregional therapy, except one patient (PD37118), who underwent a single treatment with trans-arterial chemoembolization. The PNPLA3 (rs738409) polymorphism genotype was derived from the whole genome sequencing data.

All liver specimens were scored according to the Kleiner system (Kleiner et al., Hepatology. 2005; 41(6):1313-1321) on formalin-fixed paraffin-embedded (FFPE) samples away from the fresh-frozen block used for the laser-capture microdissection. The Kleiner score, developed for NAFLD, assesses the presence of steatosis, lobular inflammation and hepatocyte ballooning to generate a cumulative NAFLD activity score (NAS). This was applied to normal and ARLD samples, in the absence of a validated scoring system for ARLD, to allow comparability between all samples of the study in terms of stage and grade of disease. The presence or absence of cellular or nodular dysplasia was assessed globally in clinical FFPE samples, as well as specifically in the fresh-frozen block used for the laser capture microsection (LCM) and sequencing.

Sample Preparation:

The protocols used for preparing liver biopsy tissue sections, laser capture microsection (LCM) and subsequent cell lysis, DNA extraction, and whole genome sequencing (WGS) were according to those previously described by Brunner et al. (Nature, 2019, 574, 538-542). Briefly, 20 μm thick tissue sections (prepared with a Leica cryotome) were fixed with 70% ethanol and stained with haematoxylin and eosin for subsequent LCM generation using a Leica Microsystems LMD 7000. The micro-dissected samples were then lysed using the Arcturus® PicoPure® DNA Extraction Kit (Thermo Fisher Scientific) following the manufacturer's instructions. DNA libraries for Illumina sequencing were prepared using a protocol optimized for low input amounts of DNA for submission to paired-end WGS. The resultant reads were mapped to the GRCh37d5 human reference genome using the BWA-MEM algorithm (Li and Durbin, Bioinformatics, 2010, 26, 589-595).

The dataset used in this study comprised 1202 genomes from 32 liver samples, including 5 normal liver controls, 10 with alcohol-related liver disease (ARLD) and 17 with NAFLD. Nine of these sample were from patients who had a synchronous HCC and underlying cirrhosis; a further 8 samples had HCC without underlying cirrhosis, including 3 hepatic resection samples from one patient with NAFLD over a 5-year timespan (samples PD37918b, PD37915b and PD37910b). Clinical and histological features of the patients showed the expected distribution for the underlying disease processes.

Laser-capture microdissection was used to isolate contiguous groups of 100-500 hepatocytes for whole genome sequencing from the liver samples. Microdissections were sequenced to an average depth of 31×. Somatic substitutions and structural variants were called according to the method described by Brunner et al. (2019), but with the use of gradient-boosted regression trees to improve the characterization and accuracy of indel calls. With a complete catalogue of somatic mutations, the phylogenetic tree structures, clone sizes, driver mutations, telomere lengths and mutational signatures were inferred.

Snv Calling:

Several steps of the SNV calling workflow that was used in this study were previously described by Brunner et a1 (2019). Basic SNV identification used the Cancer Variants through Expectation Maximization (CaVEMan) algorithm (Jones, D. et al., Curr Protoc Bioinformatics 56, 15.10.1-15.10.18 (2016)) to call single base substitution (SBS) variants, with per-patient bulk biopsies as matched normal controls. For the 2 NAFLD patients that were biopsied from the 8 anatomical liver segments, a thyroid follicle LCM sample was used as an unmatched control to call mutations. Duplicate reads and LCM library preparation-specific artefactual variants resulting from the incorrect processing of secondary cruciform DNA structures were removed with bespoke post-processing filtering. The latter filtering step was configured to consider all variants with at least two supporting sequenced DNA fragments. In the current study, the entropy metric based variant filtering step described in Brunner et a1 (2019) was replaced with a beta-binomial based filtering approach as described by Yoshida et al. (Yoshida, K. et al., Nature 578, 266-272 (2020)), which operates on the principle that authentic somatic mutations are typically over-dispersed (i.e., present in only a limited number of genomes in the set of genomes belonging to each patient), while systematic artefacts or germline variants are commonly under-dispersed, making them observable across many if not all genomes derived from the microdissections from the same patient biopsy. In this study, the number of mutation-bearing and total reads for each SNV was calculated by enumerating raw allele counts for each base (A, C, G, T) per SNV called across all microdissections on a patient-specific basis, where mutations with a dispersion estimate of ≥0.1 were considered to likely be true somatic variants. Manual inspection of a subset of final SNV calls using a genome browser was performed to ensure validity. A further check involved checking that spatially proximate microdissections as captured by histology images shared common mutations (i.e., within the same vicinity in terms of x-y space on the same tissue section, within the same cirrhotic nodule, or overlapping x-y positions on tissue sections from different z-planes).

Using the Hierarchical Dirichlet Process for the Identification of SNV Clusters:

The nonparametric Bayesian hierarchical Dirichlet process (HDP) was implemented to cluster SNVs with similar variant allele fractions (VAF) that were called across multiple microdissections for each patient biopsy. Full mathematical and implementation details of the clustering algorithm are described in Brunner et a1 (2019). This N-dimensional Dirichlet process (NDP) clustering approach was run with 10,000 burn-in iterations, followed by 5,000 posterior Gibbs sampling iterations that were used for clustering. This class of algorithm was chosen for the identification of SNV clusters since there is no requirement to arbitrarily prespecify the number of clusters to find. Instead, at each sampling iteration, there is a defined probability that mutations will be allocated to new clusters that did not exist in the previous iteration. On the other hand, clusters can also be removed in a future iteration in cases where all member mutations are assigned to other clusters. In this way, the number of SNV clusters are permitted to vary throughout the sampling chain. To avoid overly complex solutions consisting of a large number of clusters, which would increase the chance of creating uninformative ones, an upper limit of 100 SNV clusters per patient was imposed. A multithreaded version of the ECR algorithm modified from the label.switching R package (Papastamoulis, P., Journal of Statistical Software 69, (2015)) was used for rapid label switching correction. Only SNV clusters comprising a minimum of 50 unique mutations were kept for downstream analysis. Input to this algorithm included per-patient data tables consisting of the coverage and counts of each called variant per microdissection.

A small number of clusters (13 of 966, 1.34%) spanning 11 out of 32 patients (34.3%) were found to be under-split and consisted of microdissections that did not share any mutations. Such problematic microdissections were split off to form additional independent clusters. Clustering of spatially adjacent or proximate microdissections were verified for each set of patient-specific sequenced material by detailed inspection of corresponding histology images.

As the clustering algorithm assigns each mutation to one cluster based on similarity in VAFs across samples by design, the FOXO1 hotspot mutation was manually reassigned to a minimal number of clusters to account for the possibility of multiple independent acquisition events that can occur in several microdissections of a patient biopsy. Specifically, parsimonious hotspot reallocation entailed first identifying hotspot-bearing microdissections (and the SNV clusters of which they are members). Next, a minimal subset of the most phylogenetically parental clusters was identified (see methods for the inference of phylogenetic trees), where each had a maximal number of member microdissections with at least one raw read of support for the hotspot, such that all hotspot-bearing microdissections were accounted for. Importantly, although some hotspot-bearing microdissections were members of multiple clusters, the hotspot was only assigned to the cluster with the maximal number of hotspot-containing dissections in such instances. For a minority of cases, some member dissections of candidate hotspot-bearing clusters did not have any evidence of the hotspot mutation due to low coverage. In these cases, histology images were checked to determine whether spatial positioning of the dissections in question were proximate or distal to hotspot-bearing dissections within the same cluster. As additional support for the likelihood that these candidate dissections carried the hotspot, the sharing of mutations with bona fide hotspot-containing dissections was assessed.

Inference of Phylogenetic Trees:

The statistical pigeonhole principle, as described by Nik-Zainal et al. (Nik-Zainal, S. et al. The life history of 21 breast cancers. Cell 149, 994-1007 (2012)), was applied to infer phylogenetic clonal relationships between per-patient SNV clusters identified by the NDP algorithm, where each cluster is represented as a branch of a phylogenetic tree. A given cluster is considered to have strong evidence of being nested within another (i.e., sub-clonal relationship) if the fraction of cells carrying the cluster of mutations is lower in all member microdissections relative to the fraction of cells containing another cluster of mutations within the same microdissections, where the sum of their respective mutant cell fractions (CFs) is also >100%. Otherwise, if the sum of the pairwise mutant CFs is ≤100%, only weak evidence of nesting exists. In cases where only some microdissections have lower CFs of a given SNV cluster relative to another, the clusters are interpreted to be independent and not nested within one another. In the current study, only clusters with a mutant CF >0.05 are analysed, while the CF of each SNV cluster is calculated as 2 times the median VAF for each microdissection, which assumes diploidy.

Problematic SNV clusters containing microdissections that do not share any mutations with other member dissections of the same clusters were split up into new independent clusters, and were individually reassessed for phylogenetic relatedness with all other clusters within the same patient biopsy using the pigeonhole principle.

Identification of Mutations Under Selection:

To determine whether any coding mutations were under selection in non-tumorous chronic liver disease tissues, the dN/dScv method (Martincorena, I. et al., Cell 171, 1029-1041.e21 (2017)) on the gene, protein domain, codon, and hotspot levels was used to identify genes with a higher number of nonsynonymous mutations relative to the expected number from the rate of synonymous mutation acquisition. For this analysis, the unique mutations across the set of all SNV clusters was used as input, while mutations with q-values corrected for multiple hypothesis testing of <0.05 were considered to be under selection. For the identification of non-coding mutations that may be under selection, the NBR algorithm was used (Rheinbay, E. et al., Nature, 578, 102-111 (2020)).

Indel Calling:

As previously described Brunner et a1 (2019), indel calling was performed using cgpPindel (Raine, K. M. et al., Curr Protoc Bioinformatics 52, 15.7.1-15.7.12 (2015)). A naïve Bayes algorithm was used to assign each called indel to the SNV clusters identified using the NDP algorithm. As done during SNV calling, the beta-binomial over-dispersion filter was applied to the raw counts of each called indel across the set of microdissections made from each patient biopsy to further filter out artefacts, where variants with an over-dispersion value of ≥0.1 and VAF ≥0.025 were considered to likely be real.

To gain a better understanding of the characteristics of authentic indels, a ground truth set of indels that are shared between proximate microdissections per patient was first identified through a detailed review of the set of histology images corresponding to the study cohort. Several features of indels were next considered for statistical modelling including the number of homopolymers, whether an indel occurred in a repetitive HG19 region, the dispersion estimate from the beta-binomial filter, and various cgpPindel VCF fields (i.e., VAF excluding ambiguous and unknown reads, QUAL—indicates the sum of mapping qualities of anchor reads, coverage, coverage_alt—the number of variant supporting reads, the S1 score from Pindel, LEN—the variant length in base pairs, REP—the number of repeats, NB-Tum—the number of reads mapped by the primary aligner to the negative strand showing a similar indel event in the target sample, ND-Tum—the count of negative strand mapped reads from the primary aligner with or without the indel in the target sample, NP-Tum—the number of reads mapped by Pindel to the negative strand in the target sample, NR-Tum—the unique union of NP-Tum and ND-Tum, NU-Tum—the unique union of NP-Tum and NB-Tum, PB-Tum—the number of reads mapped by the primary aligner to the positive strand showing a similar indel event in the target sample, PD-Tum—the count of positive strand mapped reads from the primary aligner with or without the indel in the target sample, PP-Tum—the number of reads mapped by Pindel to the positive strand in the target sample, PR-Tum—the unique union of PP-Tum and PD-Tum, PU-Tum—the unique union of PP-Tum and PB-Tum, AMB-Tum—Ambiguous reads mapping on both the alleles with the same specificity in the target sample, UNK-Tum—Unknown reads containing mismatches in the variant position and don't align to the reference as the first hit, VT—the variant type either insertion or deletion).

These parameters were used as input to the XGBoost algorithm (Chen, T. & Guestrin, C. XGBoost: A Scalable Tree Boosting System. dl.acm.org, 785-794 (ACM, 2016). doi:10.1145/2939672.2939785) in R to construct a series of 1,000 gradient boosted regression trees (also known as classification and regression trees (CARTS)) in order to elucidate the typical attributes of likely real indels in the truth set. Manual hyperparameter tuning was performed using a minimal grid-search based approach to arrive at a learning rate (∈) of 0.003, a minimum loss reduction (γ) of 20 for leaf node partitioning, a subsampling rate of 60% of the indels and 80% of covariates in the training data for learning regression trees, and a maximum tree depth of three. Furthermore, model training was performed while optimizing a binary logistic objective function, setting the L2 regularization factor (λ) and bias to 0.5, enabling leave-one-out-cross-validation, and setting the model training metric to assess the area under the receiver operator curve (AUROC). All other hyperparameters were set to default or recommended values according to software documentation. At the end of the model training process, a binary ensemble classifier is built in which scores from a large number of shallow regression trees each with low predictive power, are combined to give high predictive power. In the so-called ensemble boosting framework, the set of regression trees are trained additively, with the initial tree (t=0) trained to directly fit the dependent variable (in this case it is whether or not an indel has features that are similar to those of the patient-specific truth set) such that a logistic loss function is minimized, while subsequent trees are trained to fit the residuals of the overall model learned up until the previous iteration (t−1). For each indel, a series of scores from the regression tree base learners were then combined in a weighted fashion to arrive at a final score representing the probability of whether or not a given indel was likely a true or artefactual variant. A model score of >0.7 was used to identify likely real indels. The predictive accuracy of the regression-tree ensemble model was assessed on a per-patient basis by evaluating the AUROC resulting from a comparison between the set of model-predicted and ground truth indels.

In a boosted regression forest model, N trees (base learner functions fₜ(xᵢ) that map predictive features of indels to model scores) are learned from a training dataset comprising predictor and dependent variable pairs {(xᵢ,yᵢ)}ᵢ₌₁ⁿ, where each tree stores a vector of leaf node scores, and its structure as a mapping function that assigns indel feature values to leaf nodes, while the overall model scores (predictions ŷᵢ) are computed as the sum of scores from each tree:

ŷᵢ^{(N)}=Σₜ₌₁^{N}fₜ(xᵢ)=ŷᵢ^{(t=1)}+∈fₜ(xᵢ) [1]

Here ∈ is the learning rate, which scales each tree's contribution to the overall model score to safeguard against overfitting to the training data.

An additive process of sequential tree structure learning is adopted for building a boosted regression forest, wherein a tree structure fₜ(xᵢ) is learned at each training iteration that minimizes the following objective function:

$${obj}^{( t )} = {\sum }_{i = 1}^{n} [ {g}_{i} {f}_{t} ( {x}_{i} ) + \frac{1}{2} {h}_{i} {f}_{t}^{2} ( {x}_{i} ) ] + Ω ( {f}_{t} ) \qquad [ 2 ]$$

$${g}_{i} = {\partial }_{{\hat{y}}_{i}^{( t - 1 )}} ℒ = \frac{( 1 - {y}_{i} ) {e}^{{\hat{y}}_{i}^{( t - 1 )}}}{1 + {e}^{{\hat{y}}_{i}^{( t - 1 )}}} - \frac{{y}_{i} {e}^{- {\hat{y}}_{i}^{( t - 1 )}}}{1 + {e}^{- {\hat{y}}_{i}^{( t - 1 )}}} \qquad [ 3 ]$$

$${h}_{i} = {\partial }_{{\hat{y}}_{i}^{( t - 1 )}}^{2} ℒ = ( 1 - {y}_{i} ) [ \frac{{e}^{{\hat{y}}_{i}^{( t - 1 )}}}{1 + {e}^{{\hat{y}}_{i}^{( t - 1 )}}} - \frac{{e}^{2 {\hat{y}}_{i}^{( t - 1 )}}}{{( 1 + {e}^{{\hat{y}}_{i}^{( t - 1 )}} )}^{2}} ] + {y}_{i} [ \frac{{e}^{- {\hat{y}}_{i}^{( t - 1 )}}}{1 + {e}^{- {\hat{y}}_{i}^{( t - 1 )}}} - \frac{{e}^{- 2 {\hat{y}}_{i}^{( t - 1 )}}}{{( 1 + {e}^{- {\hat{y}}_{i}^{( t - 1 )}} )}^{2}} ] \qquad [ 4 ]$$

$$ℒ = {y}_{i} ln ( 1 + {e}^{- {\hat{y}}_{i}^{( t - 1 )}} ) + ( 1 - {y}_{i} ) ln ( 1 + {e}^{{\hat{y}}_{i}^{( t - 1 )}} ) \qquad [ 5 ]$$

$$Ω ( {f}_{t} ) = γ T + \frac{1}{2} λ {\sum }_{j = 1}^{T} {ω}_{j}^{2} \qquad [ 6 ]$$

where,
obj^{(t)} Is the objective function to be optimized for regression tree t,
fₜ(xᵢ) Denotes regression tree t, evaluating the score of feature values xᵢ,
gᵢ Is the first order derivative of the loss function with respect to the model score from the previous iteration ŷₜ^{(t=1)},
hᵢ Is the second order derivative of the loss function with respect to the model scores from the previous iteration ŷₜ^{(t=1)},
[symbol] Is the logistic loss function, which describes how well the model fits the training data,

Ω(fₜ) Is a regularization term describing the complexity of regression tree t, defined as a function of T leaves and the L2 norm of a vector of scores ωⱼ of indels assigned to leaf node j.

A logistic loss function was used in this study since the binary classification of likely real or artefactual indels was of interest. In order to approximate the logistic loss, a second order Taylor expansion is used. Thus, each training iteration starts with the calculation of gᵢ and hᵢ.

Letting Iⱼ={i|q(xᵢ)=j} be the set of indels assigned to leaf node j, fₜ(xᵢ)=ωⱼ can be used to indicate that assigning indel i to node j will result in a score of ωⱼ for that indel, which allows for the objective function to take the following form after some algebraic manipulation:

$${obj}^{( t )} = {\sum }_{j = 1}^{T} [ {G}_{j} {ω}_{j} + \frac{1}{2} ( {H}_{j} + λ ) {ω}_{j}^{2} ] + γ T \qquad [ 7 ]$$

$${G}_{j} = {\sum }_{i ∈ {I}_{j}} {g}_{i} \qquad [ 8 ]$$

$${H}_{j} = {\sum }_{i ∈ {I}_{j}} {h}_{i} \qquad [ 9 ]$$

Setting obj^{(t)}=0 and taking the first derivative with respect to ωⱼ allows us to solve for the optimal leaf node weight ωⱼ*:

$${ω}_{j}^{*} = - \frac{{G}_{j}}{{H}_{j} + λ} \qquad [ 10 ]$$

Substituting [10] back into [7] gives the objective function value obj* as a measure of optimality of a given tree structure, where lower values are more favourable:

$${obj}^{*} = - \frac{1}{2} {\sum }_{j = 1}^{T} \frac{{G}_{j}^{2}}{{H}_{j} + λ} + γ T \qquad [ 11 ]$$

As there are often an innumerable number of possible node configurations for any given tree, it is not practical to compute obj* for every possible tree structure in order to find the one with the optimum value. Instead, a greedy approach was taken to grow a tree one bifurcation at a time, starting with a minimal tree with zero depth. Then for each leaf node in the regression tree t being grown, the following gain metric was calculated, which evaluates the change in the objective function value after each new split is introduced. In this way, a tree was grown such that gain is maximized.

$$Gain = \frac{1}{2} [ \frac{{G}_{L}^{2}}{{H}_{L} + λ} + \frac{{G}_{R}^{2}}{{H}_{R} + λ} - \frac{{( {G}_{L} + {G}_{R} )}^{2}}{{H}_{L} + {H}_{R} + λ} ] - γ \qquad [ 12 ]$$

Here,

$$\frac{{G}_{L}^{2}}{{H}_{L} + λ} and \frac{{G}_{R}^{2}}{{H}_{R} + λ}$$

denote the scores on the newly added left and right child nodes, respectively, while

$$\frac{{( {G}_{L} + {G}_{R} )}^{2}}{{H}_{L} + {H}_{R} + λ}$$

denotes the score on the parent node where the new split was considered. Together, these three bracketed terms represent the training loss reduction. Lastly, γ represents a regularization term that accounts for the cost of adding complexity to the tree structure when introducing a new split. Moreover, γ also serves as the minimum amount of loss reduction required for partitioning a given leaf node of a given regression tree, where a larger value would result in a tree having fewer new branch points added. This is because, the following condition would be true more frequently:

$$\frac{1}{2} [ \frac{{G}_{L}^{2}}{{H}_{L} + λ} + \frac{{G}_{R}^{2}}{{H}_{R} + λ} - \frac{{( {G}_{L} + {G}_{R} )}^{2}}{{H}_{L} + {H}_{R} + λ} ]$$

<γ, which would result in unfavorable negative gains, indicating that in such cases, branching would lead to a suboptimal tree structure and should therefore not occur. In this way, the calculated gain at each leaf node of a tree can be used to determine which new bifurcations could be generated to maximize loss reduction. To identify an optimal split for a particular feature at a given leaf node, one can systematically explore thresholds for grouping indels according to their feature values in order to calculate the sum of gains for each pair of groupings (i.e., indels with feature values less than or greater than some threshold value). By doing this, it is possible to identify the optimal threshold value that results in maximum loss reduction and therefore gain. Using this approach, optimal splits were identified iteratively for all indel features considered by the model until a prespecified tree depth was reached, at which point, an optimal tree structure fₜ(xᵢ) was identified that was then added to the overall model as defined in [1] before proceeding to learn another tree structure in the next training iteration. The algorithm exits only when a predefined number of trees has been learned.

It is notable that the ground truth set does not capture proximately shared indels that are genuinely present at loci where coverage is low, or indels that are private to particular microdissections. Indels in this category were found to often possess attributes that are similar to the ground truth variants and therefore have high model scores, which were validated to likely be real through manual review using a genome browser.

The importance of each model feature was evaluated using Shapley values, which is a concept rooted in game theory that provides a method for the determination of contributions from multiple contributors to a given result in a provably fair manner (see Shapley, L. S. 17. A Value for n-Person Games. Contributions to the Theory of Games (AM-28), Volume II 307-318 (Princeton University Press, 2016). doi:10.1515/9781400881970-018/html and Lundberg, S. M. & Lee, S.-I. A Unified Approach to Interpreting Model Predictions. 4765-4774 (2017)). Numerical values were calculated based on a weighted average of differences in model scores with and without a given predictive feature over the set of models that together accounts for all possible permutations of feature groupings, excluding the feature being evaluated. Thus, the Shapley value of a feature represents the mean contribution (effect) of that feature's value to the overall model score, which was calculated as follows:

$${ϕ}_{i} ( p ) = {\sum }_{S ⊆ N / i} \frac{\vert S \vert ! ( n - \vert S \vert - 1 ) !}{n !} ( p ( S ⋃ i ) - p ( S ) ) \qquad [ 13 ]$$

Where,
ϕᵢ(p) Is the Shapley value of feature i for model prediction p,
S⊆N/i Represents all possible sets (S) of feature groupings, excluding feature i,
   |S|! Represents the number of permutations of features in set S,
(n−|S|−1)! Represents the number of permutations of features not in set S out of the total of n features considered,
p(S∪i) Is the model prediction p with feature i,
p(S) Is the model prediction p without feature i.

In the case of regression forests, the Shapley value of a given predictive feature was the weighted sum of Shapley values corresponding to that feature in the ensemble of constituent trees. Here, the SHAP (SHapley Additive exPlanations) implementation designed specifically for regression-tree based models was used, which more rapidly calculates Shapley values based on the subset of features present in each regression tree in the ensemble (Lundberg & Lee, 2017). For a particular prediction of whether a given indel was likely real or artefactual, the overall model score was broken down as the sum of Shapley values (contributions to the prediction) of each predictive feature plus the baseline (average) model score calculated from the training data. Presence or absence of features that are highly predictive of whether an indel was likely real or artefactual result in large changes in model scores. High values of such influential features either contribute positively or negatively to the scores, are typically associated with Shapley values of greater or less than zero, and correspond to traits of true or false indels, respectively. Conversely, features with a negligible effect on the overall model scores have near-zero Shapley values.

Structural Variant Calling:

Structural variants (SVs) including deletions, inversions, tandem-duplications, and translocations affecting large genomic segments were called using the BRASS (breakpoint via assembly) algorithm (Campbell, P. J. et al., Nat. Genet. 40, 722-729 (2008))(https://github.com/cancerit/BRASS). A three-step process was next used to filter out likely artefactual SVs called by BRASS. First, a custom pipeline was developed that identifies and removes artefactual variants that were introduced by the LCM library preparation protocol, based on comparing the SV events detected in each microdissection with those present in a panel of corresponding normal bulk control samples. Second, detailed manual review of all remaining SVs was conducted using a genome browser and variant annotations. Finally, similar to the sanity checks that SNVs and indels were subjected to, the presence of each SV was checked among proximate microdissections where possible, where it is expected that real variants would be shared by such clusters of dissections.

More complex genomic rearrangement events such as chromothripsis (Stephens, P. J. et al., Cell 144, 27-40 (2011)), in which one or more chromosomes are shattered into as many as thousands of pieces that are subsequently fused back together in a disordered fashion, were additionally identified through detailed manual review of the final set of SVs.

Calculation of Clonal Areas:

The exact spatial positions of 1,202 microdissections were captured in a series of microscopy images taken with the LMD7 laser microdissection in-built camera. The Cartesian coordinates of the outer edge of each dissection was extracted using the Canny method (https://uk.mathworks.com/matlabcentral/fileexchange/46859-canny-edge-detection) as implemented in the edge function from the Image Processing MATLAB toolbox. This resulted in a set of x-y coordinates per microdissection, which were manually annotated to correspond to their respective WGS profiles. Next, the single-nucleotide variant (SNV) clusters were processed individually starting with the identification of their respective member microdissections that bear mutations assigned to each cluster. For each SNV cluster, the mutant CF (i.e., 2×median VAF) was used to adjust each member dissection's x-y coordinates on the tissue section image to more accurately reflect genetic clone size. A minimal ellipsoid convex hull was subsequently drawn to encompass the adjusted spatial coordinates of each member microdissection of a given SNV cluster, before merging the resultant polygons into a single entity representing the corresponding clone area. Clone area was initially computed in terms of squared pixels, before a pixel to micron conversion was applied to translate the units to squared microns. For this, multiplicative conversion factors were calculated by first generating images of scale indicators overlaid atop high-resolution scanned histology images of tissue sections. This was done using the NDP.view2 NanoZoomer Digital Pathology slide scanner image viewing software from Hamamatsu Photonics. The scale indicator images were then loaded into the R statistical programming environment using the magick image processing package (https://cran.r-project.org/web/packages/magick/index.html) in order to determine the exact number of pixels per millimetre for each tissue section image. In this study, only microdissections that contributed mutations with VAF≥0.05 were included in the clone size calculation.

Counting Alleles and Indels

Raw allele counts of each base (A, C, G, T) and total coverage of unfiltered reads were enumerated using alleleCount software for SBS-type variants to generate input for the beta-binomial based filter, while setting the minimum mapping and base quality to 30 and 25, respectively (http://cancerit.github.io/alleleCount/). For input to NDP clustering and for counts of indels, cgpVAF software was used (https://github.com/cancerit/vafCorrect).

Allele counter software was used to determine the number of unfiltered raw counts of each base directly from the bam files of both LCM microdissections and bulk control samples. Stacked barplots were generated from these count data for each patient found to carry the FOXO1 S22W hotspot driver mutation to visualize its distribution of across clones and bulk control (if available).

Clone Size Comparison:

The clone areas (μm²) were compared between hepatocytes that carried driver mutations found in this study and those that did not. Specifically, for each driver mutation, clones wild-type for the driver mutation were uniformly and randomly sampled from each donor bearing the mutation so that the clone areas (weighted by the clone's number of mutations) between the number of mutated clones from each donor could be compared to an equivalent number of wild-type clones that were randomly selected from each corresponding donor. The comparison of clone areas was conducted using the ggstatsplot R package, where the Bonferroni method of multiple hypothesis testing correction to p-values was applied, while the default Mann-Whitney U test for nonparametric pairwise comparisons was used (Patil, I., The Journal of Open Source Software 6, 1-5 (2021)).

Liver Mass Estimation:

Several assumptions were made in the calculation of the grams of hepatocytes carrying each of the driver mutations identified in this study: (1) the majority cell type composing samples are hepatocytes (Schulze, R. J. et al., J Cell Biol 218, 2096-2112 (2019)), where driver mutations occurred in diploid genomic regions, and thus mutant hepatocyte fraction=2×driver allele frequency, (2) each LCM microdissection was estimated to comprise of 100 to 500 hepatocytes, (3) there are 1.16×10⁸ hepatocytes per gram of a typical 1.5 kg human liver (see Lipscomb, J. C. et al., Toxicol Appl Pharmacol 152, 376-387 (1998) and Sohlenius-Sternbeck, A.-K., Toxicol In Vitro 20, 1582-1586 (2006)). For each donor in our study, the liver-wide mass (grams) of mutated hepatocytes was inferred for each driver mutation by first calculating the area (pixels) of all sequenced LCM dissections using histology images. Since it was estimated that each dissection contained between 100 to 500 hepatocytes, a linear fit was performed using the R linMap function to map all LCM cut areas within this range, effectively estimating the number of hepatocytes composing each LCM cut. The variant allele frequency of each driver mutation was then used to infer the fraction of mutant-bearing cells in each LCM dissection. Next, the proportion of sequenced material per donor containing each driver was calculated by summing estimates from all donor-specific sequenced LCM cuts. These donor-level estimates were then used to approximate the proportion of liver cells carrying each driver based on the estimated number of hepatocytes in a typical human liver. These values, were then ultimately used to estimate the number of grams of liver that contained each driver for each donor, assuming a typical human liver weighs 1.5 kg.

Using HDP for the Extraction of Mutational Signatures:

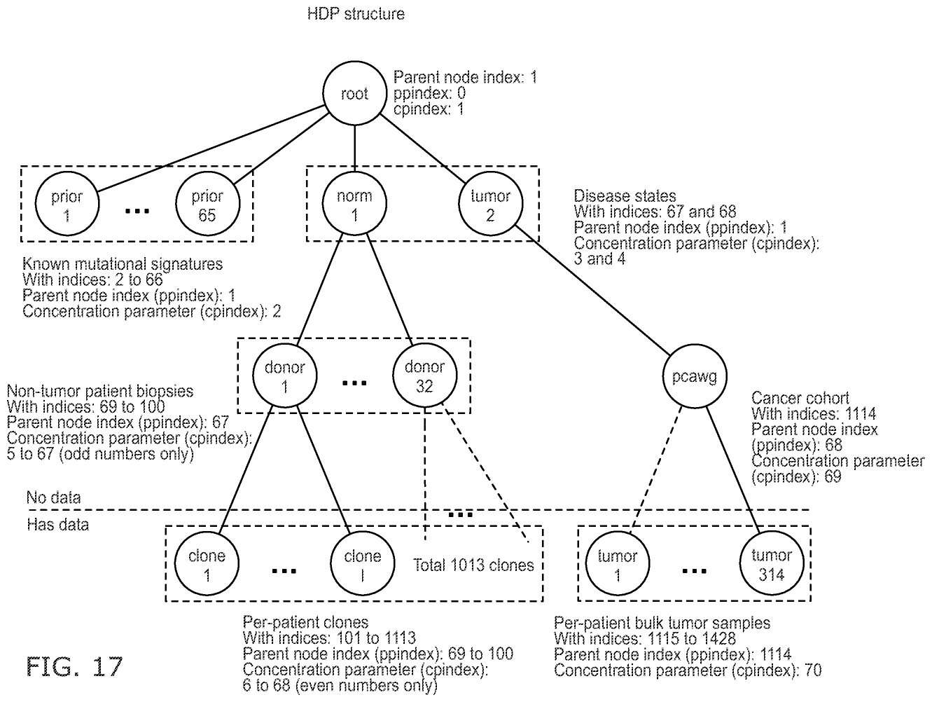

The HDP algorithm as implemented in the HDP R package (https://github.com/nicolaroberts/hdp), was used to extract mutational signatures composing the set of SBSs called in each of the 1,013 SNV clusters identified in normal liver and chronic liver disease samples. Input to the algorithm consisted of a matrix of mutation counts per SNV cluster for each of the mutation categories, which in this case consisted of 192 trinucleotide mutational contexts (generated using the SigProfilerMatrixGenerator software (Bergstrom, E. N. et al. BMC Genomics 20, 1-12 (2019)) as defined by the six SBS types (C>A, C>G, C>T, T>A, T>C, T>G), with each further defined by all possible combinations of bases (A, C, T, G) flanking the mutated base (3′ and 5′), for the transcribed and un-transcribed strands. A reference catalogue of 65 previously identified 192-context-based mutational signatures from the PanCancer Analysis of Whole Genomes (PCAWG) study was used as prior information (Alexandrov, L. B. et al., Nature 578, 94-101 (2020)). Signatures that had been previously observed in hepatocellular carcinoma (HCC) samples (i.e., SBS1, SBS3, SBS4, SBS5, SBS6, SBS9, SBS12, SBS14, SBS16, SBS17a, SBS17b, SBS18, SBS19, SBS22, SBS23, SBS24, SBS26, SBS28, SBS29, SBS30, SBS31, SBS35, SBS37, SBS40) were assigned the default weighting of 1000 pseudocounts during analysis to facilitate the extraction of known liver-relevant signatures. The remaining prior signatures were assigned a lower weighting of 100 so as to not rule them out completely in the analysis. By design, HDP allows for a degree of de novo discovery of novel mutational signatures that are dissimilar to the set of known signatures supplied as prior information. To further guide the extraction of liver-related mutational signatures, 314 HCC WGS profiles were also included in the analysis. A burn-in of 100,000 iterations was used, followed by 500 posterior Gibbs sampling iterations that were performed 500 iterations apart, while adjusting the concentration parameter, which controls the degree of cluster merging versus splitting (lower vs higher values, respectively), a total of five times at each iteration, and starting with 70 clusters where mutations are initially randomly assigned. A long burn-in combined with widely spaced collection intervals of posterior samples was chosen so as to minimize the chance of violating the assumption of independent posterior sampling. Furthermore, 70 initial clusters were used to ensure that the starting distribution of mutations were spread over all 65 prior reference signatures plus a few additional clusters to promote the extraction of novel mutational signatures beyond the set of given priors. At each iteration, each mutation is assigned to a cluster with a high proportion of mutations in the same mutation category, sample, or parent node. Clusters with cosine similarity >0.9 are merged as per the default settings, while residual mutations unassigned to the set of extracted signatures due to uncertain cluster membership are grouped together to represent the percentage of data that is unexplained by the resultant model. A cosine similarity of >0.8 (as computed using the philentropy R package (Drost, H.-G., J. Open Source Softw. 3, 765 (2018)) along with manual inspection was used to determine whether any of the extracted signatures match any of the known priors, where a slightly lower similarity threshold was used to account for possible variations of the reference signatures. A computational deconvolution method known as the Perturbation model (Qiao, W. et al., PLoS Comput Biol 8, e1002838 (2012)) was used to estimate the percent contribution of PCAWG mutational signatures composing each of the HDP-extracted signatures as a secondary measure of similarity between known and extracted signatures. Extracted signatures that were unique enough such that no close match to any prior signature can be assigned with reasonable certainty were considered novel. For this analysis, six independent posterior sampling chains were executed concurrently for gauging convergence to stable cluster assignments for all mutations, where random seeds of 1-, 2-, 3-, 4-, 5-, and 6-million were assigned, respectively. The overall HDP node structure including the concentration parameter settings used for signature extraction is outlined in FIG. 17.

In FIG. 16, there are shown details of a new mutational signature that was noted by the inventors. In FIG. 16B, there is shown the variability in activity between nearby clones within the same liver sample.

Patients were followed or a period of several years. For certain patients, resection samples were taken several times over a period of year. FIG. 16C shows 3 hepatic resection samples from one patient over a 5-year timespan. Further details of the mutations in each biopsy are shown in FIGS. 16D, 16E and 16F

In FIGS. 16G, 16H and 161, there are shown the distribution of the signatures in samples of normal liver cells, ARLD-affected cells, NAFLD-affected cells and in 2 patients with NAFLD with all 8 anatomic segments sampled.

The inventors noted that in one patient with NAFLD, the signature was absent from the first liver resection specimen but progressively increased in intensity over the 5 years of the study (FIG. 16D-F). Thus, the signature became progressively more pronounced as liver disease progressed, possibly linked to worsening impairment of xenobiotic metabolism.

Using SigProfiler for the Extraction of Mutational Signatures:

The SigProfilerExtractor python package (Alexandrov, L. B. et al., Nature 578, 94-101 (2020)) (https://github.com/AlexandrovLab/SigProfilerExtractor), which is based on the non-negative matrix factorization algorithm, served as an alternative means for mutational signature identification. The algorithm was configured to identify 15 mutational signatures and run with 1,000 iterations. Comparison of HDP and SigProfiler extracted 192 trinucleotide context signatures was performed by evaluating the cosine similarity metric, where a value of >0.8 was deemed to indicate that a given pair of signatures were the same or slightly different versions of each other.

FOXO1-eGFP Imaging and High-Content Analyses:

High-content and live cell analyses of FOXO1-eGFP expressing cells, counter-stained with Hoechst 33342 and SPY-555-Actin (Spirochrome), were conducted on an Operetta CLS system using a 20× air NA=0.4 objective. Images of fixed cells were analysed using Harmony software (PerkinElmer). The full analysis sequences were obtained. Briefly; any non-cellular material (e.g. bright areas caused by coverslip edges) were removed; nuclei were segmented from DAPI fluorescence; a 9 pixel-wide cytoplasmic ring from around each nucleus was segmented from GFP fluorescence; and a background region was sampled from any cell-free areas 120-150 pixels away from any nucleus. Nuclei were filtered from fragments or other non-cell small objects by setting thresholds on nuclear area, roundness, and width:length ratio. Mean nuclear, cytoplasmic and background GFP fluorescence intensities were measured, and from these the nuclear:cytoplasmic ratio was calculated for each cell using background-subtracted values. The log₁₀ of these values was taken.

For live cells a similar analysis was carried out using CellProfiler (McQuin, C. et al., PLOS Biology 16, e2005970 (2018)). The full analysis sequence was obtained. Briefly, illumination correction images were calculated for both GFP and Hoechst channels by polynomial fit, and subtracted; nuclei were segmented from the Hoechst images; cytoplasm was segmented from the GFP signal, with a 9 pixel-wide ring around the nucleus used to restrict the measurement to the perinuclear region; mean nuclear and cytoplasmic GFP intensities were measured; and the nuclei were tracked through the time series. Nuclear:cytoplasmic ratios were calculated and the login of these values was taken.

Results from the live cells are displayed as the median ±variance of pooled data from four wells, each with 8 fields of view giving 1000-2000 cells analysed per well, a total of 6000-7500 cells per condition.

Protein Expression by Immunoblotting:

Immunoblotting, on SDS-PAGE gels was performed as reported in by Hoare et al. (Hoare, M. et al., Nat. Cell Biol. 18, 979-992 (2016)) using the following antibodies: anti-β-Actin (Sigma, A5441, 1:5000); anti-Akt (Cell Signaling, 2938, 1:1000); anti-phospho-Akt (T308) (Cell Signaling, 4056, 1:1000); anti-GFP (Abcam, ab6556, 1:1000); anti-FOXO1 (Cell Signaling, 2880, 1:1000); anti-phospho-FOXO1 (T24) (Cell Signaling, (9464, 1:1000).

Metabolomics:

HepG2 cells expressing either wild-type FOXO1-eGFP or FOXO1^{S22W}-eGFP were cultured overnight in serum-free media before stimulation with or without 100 nM insulin for 3 hours prior to harvesting. Cells were washed in PBS, before extraction and lysis in 50% methanol, 30% acetonitrile (both Fisher), 20% ultrapure water and 5 μM Valine d8 (internal control, CK isotopes) on dry ice. The cellular lysate supernatant was then stored at −80° C. until LC-MS based metabolomics was performed.

HILIC chromatographic separation of metabolites was achieved using a Millipore Sequant ZIC-pHILIC analytical column (5 μm, 2.1×150 mm) equipped with a 2.1×20 mm guard column (both 5 mm particle size) with a binary solvent system. Solvent A was 20 mM ammonium carbonate, 0.05% ammonium hydroxide; Solvent B was acetonitrile. The column oven and autosampler tray were held at 40° C. and 4° C., respectively. The chromatographic gradient was run at a flow rate of 0.200 mL/min as follows: 0-2 min: 80% B; 2-17 min: linear gradient from 80% B to 20% B; 17-17.1 min: linear gradient from 20% B to 80% B; 17.1-22.5 min: hold at 80% B. Samples were randomized and analysed with LC-MS in a blinded manner with an injection volume was 5 μl. Pooled samples were generated from an equal mixture of all individual samples and analysed interspersed at regular intervals within sample sequence as a quality control.

Metabolites were measured with a Thermo Scientific Q Exactive Hybrid Quadrupole-Orbitrap Mass spectrometer (HRMS) coupled to a Dionex Ultimate 3000 UHPLC. The mass spectrometer was operated in full-scan, polarity-switching mode, with the spray voltage set to +4.5 kV/−3.5 kV, the heated capillary held at 320° C., and the auxiliary gas heater held at 280° C. The sheath gas flow was set to 25 units, the auxiliary gas flow was set to 15 units, and the sweep gas flow was set to 0 unit. HRMS data acquisition was performed in a range of m/z=70-900, with the resolution set at 70,000, the AGC target at 1×10⁶, and the maximum injection time (Max IT) at 120 ms. Metabolite identities were confirmed using two parameters: (1) precursor ion m/z was matched within 5 ppm of theoretical mass predicted by the chemical formula; (2) the retention time of metabolites was within 5% of the retention time of a purified standard run with the same chromatographic method. Chromatogram review and peak area integration were performed using the Thermo Fisher software Tracefinder 5.0 and the peak area for each detected metabolite was normalized against the total ion count (TIC) of that sample to correct any variations introduced from sample handling through instrument analysis. The normalized areas were used as variables for further statistical data analysis.

Statistical analysis of metabolomics data was performed using linear models with insulin (with or without) and FOXO1 status (mutant or wild-type) as the predictive variables, and normalised metabolite levels as the dependent variable. Likelihood ratio tests were used to generate p values, which were then corrected for multiple hypothesis testing using the Benjamini-Hochberg method. A threshold of q<0.01 was used for significance.

HepG2 Cell Lines: RNA-Sequencing Data Pre-Processing:

HepG2 cell line samples (n=30) were subjected to 2 lanes of 150 base pair paired end RNA sequencing using the Illumina HiSeq 4000 platform. The human reference genome used was hs37d5 from the 1000 Genomes Project, with gene annotations based on Ensembl release 75 data. Adaptors and low-quality reads were removed using Trim Galore (https://github.com/FelixKrueger/TrimGalore) with the following parameters: -q 20 --fastqc --paired --stringency 1 --length 20 -e 0.1. The Spliced Transcripts Alignment to a Reference (STAR) aligner was used to map the raw sequencing reads to the GRCh37 (hg19) human reference genome (Dobin, A. et al., Bioinformatics 29, 15-21 (2013)). Substitutions were called using HaplotypeCaller (Poplin, R. et al., bioRxiv 1-22 (2018). doi:10.1101/201178). The featureCounts software (Liao, Y., et al., Bioinformatics 30, 923-930 (2014).) was used to summarize gene expression values, while the cpm function from the EdgeR R package was used to normalize the data into the log counts per million scale (Robinson, M. D. et al., Bioinformatics 26, 139-140 (2010)). All heatmaps were generated using the pheatmap R package (https://cran.r-project.org/web/packages/pheatmap/index.html).

Gene Set Enrichment Analysis:

Gene set enrichment analysis (GSEA v3.0) (Subramanian, A. et al., PNAS 102, 15545-15550 (2005)) was performed using a pre-ranked list of genes, 2000 permutations, and all Gene Ontology and Reactome associated gene sets that had at most 500 genes (June_01_2021 version, downloaded from http://download.baderlab.org/EM_Genesets/). Specifically, for each gene, two linear models were built using the Im function in the R statistical programming environment, one with both FOXO1 driver and insulin status (i.e., either present or absent) as independent variables, while the other model only included insulin status. The dependent variable in both models is the expression of the gene in the model. The likelihood ratio test was then used to calculate a p-value between each pair of nested models per gene. This p-value was subsequently multiplied by the sign of the regression coefficient for mutation status in the model with the driver for each gene. Finally, the gene list was ranked according to this set of p-values as follows: −(≈0) . . . −0.05 . . . −0.99 . . . 0.99 . . . 0.05 . . . ≈0, where genes at the bottom of the list are expected to be the most associated with the presence of the FOXO1 driver, while accounting for the effects of insulin status.

### Example 1: Identification of Driver Mutations in Liver Samples

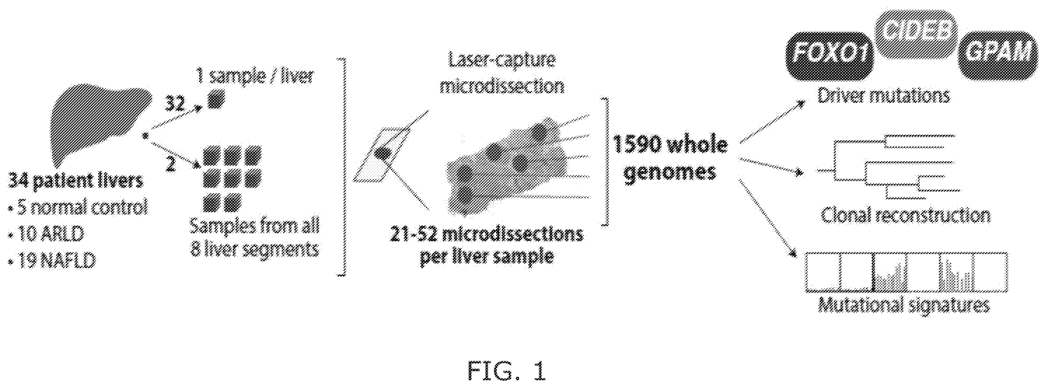

Brunner et al. (Nature, 2019, 574(7779):538-542) sequenced 482 whole genomes from healthy and diseased liver, but lacked statistical power for definitive identification of genes under selective pressure. To improve power for detecting driver mutations (i.e. mutations that confer a selective advantage on a cell) over the method used by Brunner et al., the data from Brunner et a1 was used together with an additional 1108 whole genome sequences from liver samples. These samples were predominantly from patients with NAFLD, but varied by disease severity. The hierarchical experimental design shown in FIG. 1 was used to analyse the samples. In brief, for each ˜1 cm³ of liver tissue sample, 21-52 separate microdissections were captured, each of which contained 100-500 hepatocytes. In two patients with NAFLD, separate samples were taken from all 8 Couinaud anatomical segments of their explanted liver, and 22-28 microdissections from each segment were sequenced. This hierarchical experimental design enabled the quantification of the heterogeneity of somatic mutations across individual clones, anatomical segments and patients, using specialised statistical analyses to manage non-independence arising from multiple genomes per patient.

When combined with the data from Brunner et a1, the dataset comprised 1590 genomes from 34 liver samples, including 5 normal liver controls with no prior neoadjuvant therapy, 10 with alcohol-related liver disease (ARLD) and 19 with NAFLD. All patients with ARLD or NAFLD had HCC, liver failure or both and tissues were derived from hepatic resection or transplantation. Overall, 9 samples were from patients who had a synchronous HCC and underlying cirrhosis; a further 8 samples had HCC without underlying cirrhosis, including 3 hepatic resection samples from one patient over a 5-year timespan. All samples underwent central histological review by specialist hepatopathologists, and the histological and clinical features of the patients matched those expected for the underlying disease processes. Microdissections were sequenced to an average depth of 31×.

To identify protein-coding mutations under positive selection, the dNdScv algorithm (Martinocorena et al., Cell, 2017, 171, 1029-1041) was used to screen for a significant excess of non-synonymous mutations in a gene.

To search for non-coding driver mutations, promoters, enhancers, 5′-UTRs, 3′-UTRs long non-coding RNA genes and microRNAs were screened using the NBR algorithm (Rheinbay et al., Nature, 2020, 578, 102-111).

Results:

Across all protein-coding genes, the genes ACVR2A, CLCN5, FOXO1, CIDEB, ALB, GPAM and TNRC6B genes were found to have a significant recurrence of mutations (q<0.05, i.e. FOXO1 (q<2×10⁻¹⁶), CIDEB (q<2×10⁻¹⁶), ACVR2A (q=7×10⁻⁹), ALB (8×10⁻¹⁰), GPAM (q=1×10⁻⁵) and TNRC6B (q=0.04)) after correction for multiple hypothesis testing (Martincorena et al. Cell 171, 1029-1041 (2017). Non-coding mutations were identified in NEAT1. Details of the mutated genes are set out below:

a) Coding Mutations:

FOXO1:

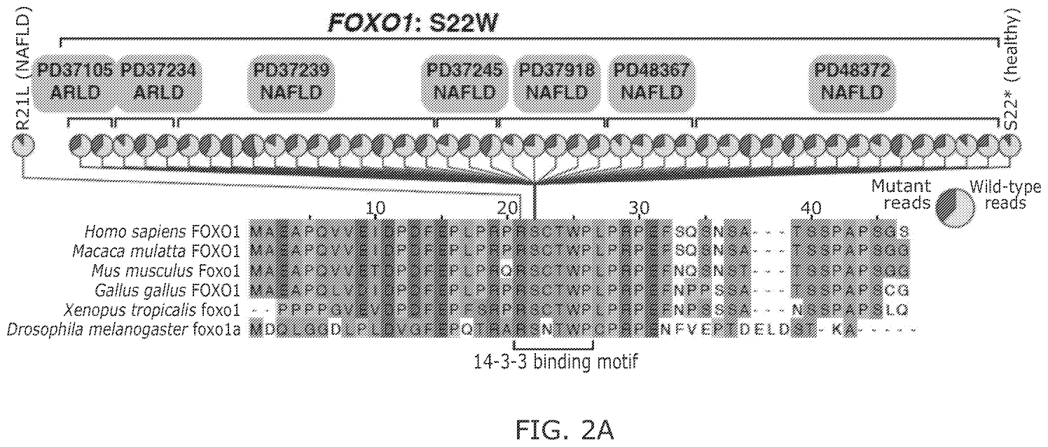

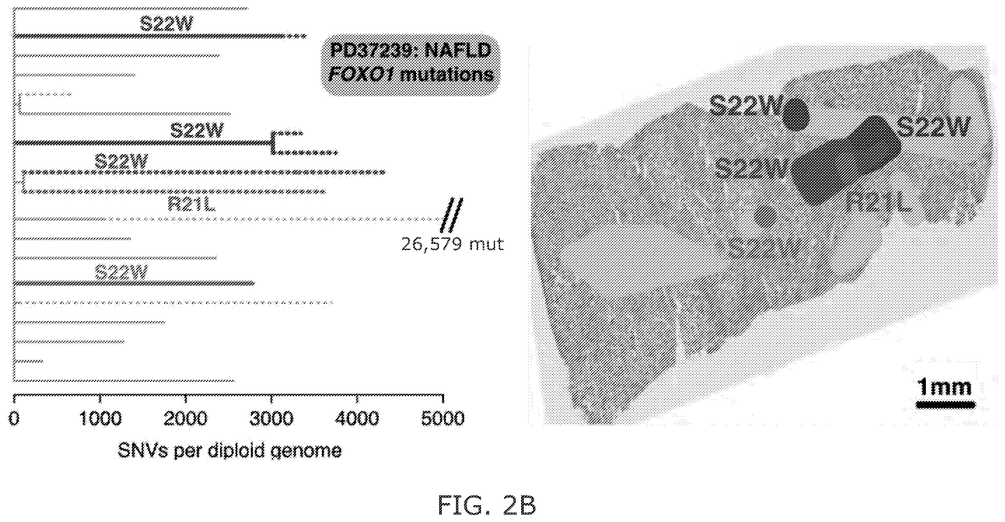

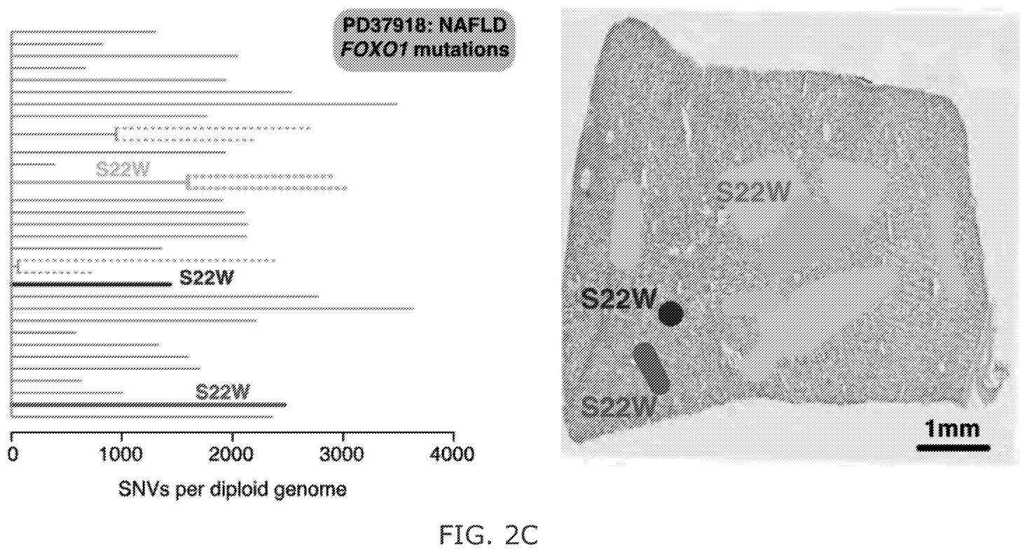

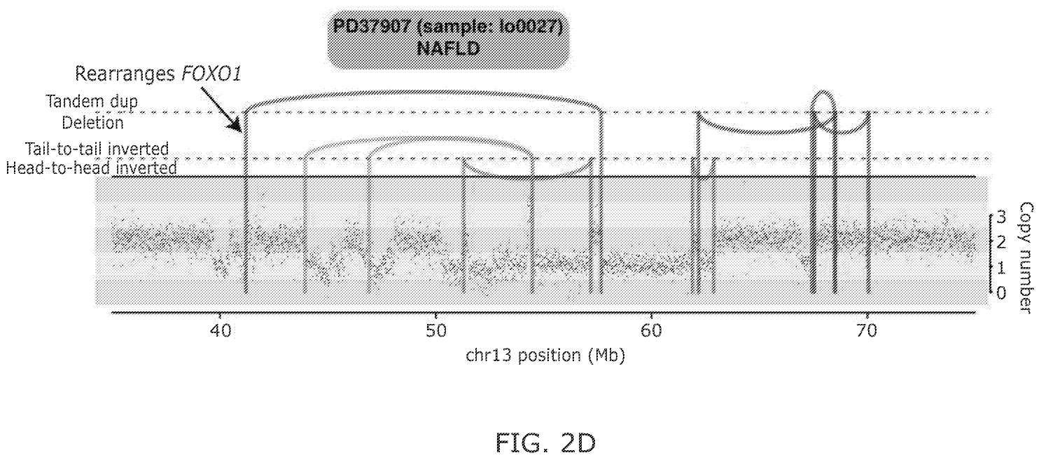

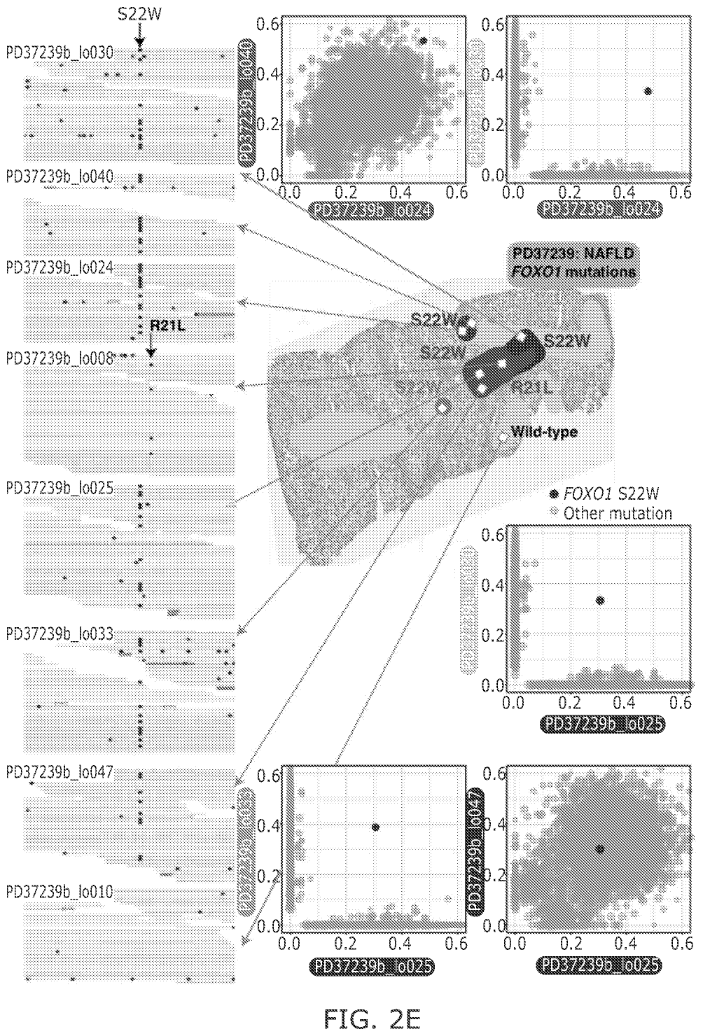

The mutations identified in the FOXO1 gene were found to be a highly significant excess of missense mutations (q<2×10⁻¹⁶). Overall, 26 separate clones were identified that had acquired independent FOXO1 mutations—these were distributed among 45 individual microdissections from 8 patients. Of these, 24 clones contained an identical base change predicted to generate an S22W amino acid substitution (FIG. 2A). The other two mutations would generate an R21L substitution and an S22* nonsense mutation. The other two mutations were predicted to generate an R21L substitution and an S22* nonsense mutation. The latter was in a single microdissection from a normal liver sample, and its biological significance is uncertain. S22W mutations were only seen in patients with ARLD or NAFLD. A somatic structural variant within the first intron of FOXO1 gene was observed in a microdissection from a patient with NAFLD—this occurred in the setting of a chromothripsis event affecting chromosome 13 (FIG. 2D).

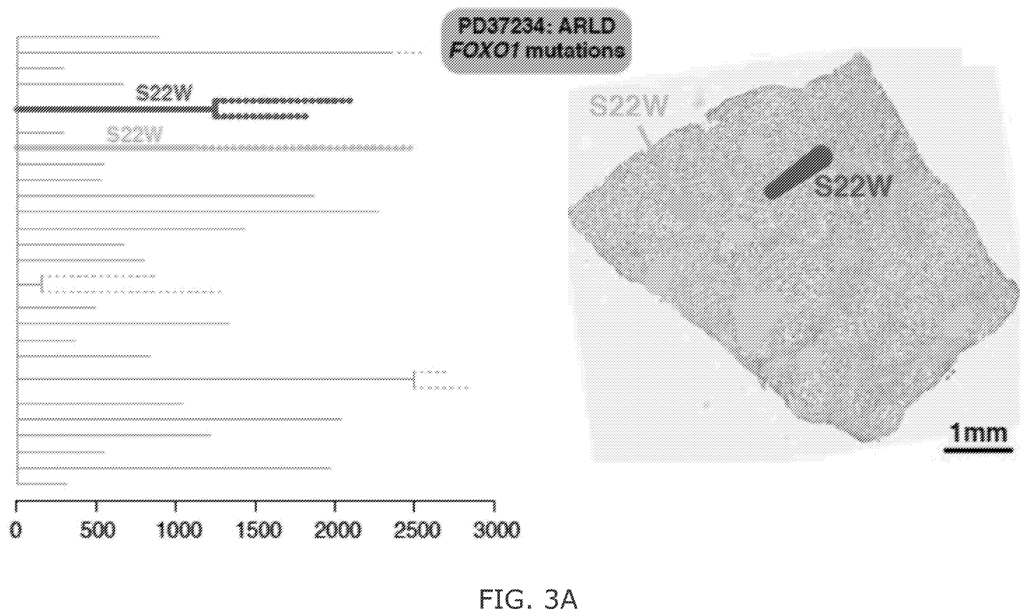

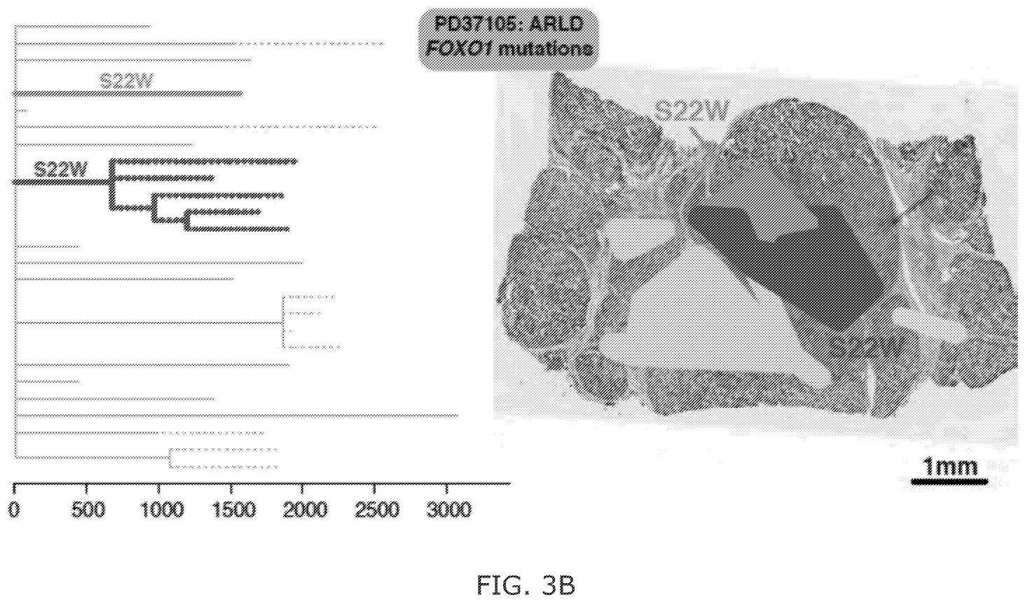

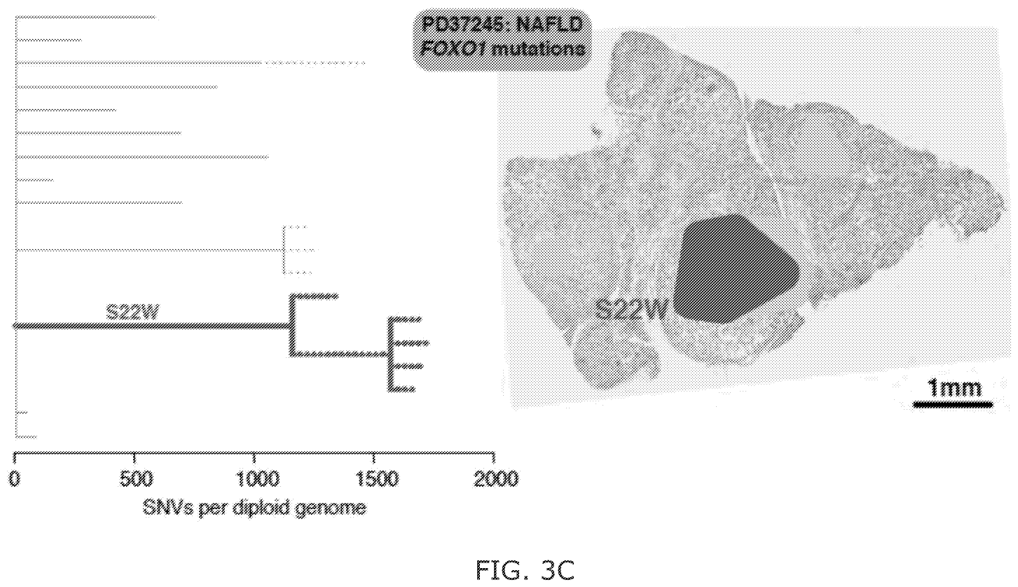

Of the 7 patients with FOXO1 S22W mutations, 6 had clear evidence for multiple independent acquisitions of the mutation in different clones—that is, convergent evolution (FIG. 3C).

In the two patients in whom all 8 anatomical segments of the liver were sampled, FOXO1 S22W mutations were found in 3 different segments in one patient and 4 segments in the other. Furthermore, even within a single segment, there were multiple, independently acquired FOXO1 mutations, such that one of these two patients had 9 independent clones with FOXO1 S22W among regions sampled.

Of the patients in whom samples from a single segment were analysed, one had 5 independent acquisitions of FOXO1 missense mutations within ˜1 cm³; a further patient had 3 separate occurrences; and 2 patients had 2 separate acquisitions.

Layering the phylogenetic trees for these patients onto the histology of the corresponding liver samples revealed that FOXO1 mutations acquired through parallel evolution were located in geographically distinct areas, typically separated by intervening regions wild-type for FOXO1 (FIG. 3A-C).

CIDEB:

It was observed that there was a significant excess of somatic mutations in CIDEB (q<2×10⁻¹⁶), one of three CIDE-family genes involved in lipid metabolism in liver and adipose tissue. CIDEB is the major family member active in hepatocytes, and knock-out mouse models show resistance to dietary steatohepatitis and increased insulin sensitivity (Li et al. Diabetes, 2007, 56, 2523-2532. These proteins regulate fusion of intracellular lipid droplets, mediated by the formation of homodimers between CIDE proteins on different droplets (Schulze, R. J. et al., J Cell Biol 218, 2096-2112 (2019) and Lipscomb, J. C. et al., Toxicol Appl Pharmacol 152, 376-387 (1998)). Homodimerisation occurs through electrostatic contacts between positively charged residues on the CIDE protein from one lipid droplet and negatively charged residues on the other.

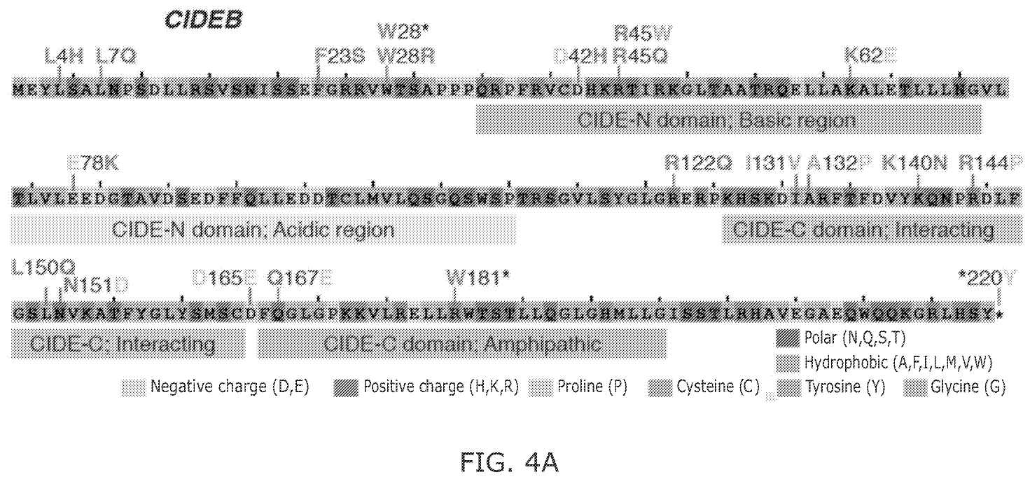

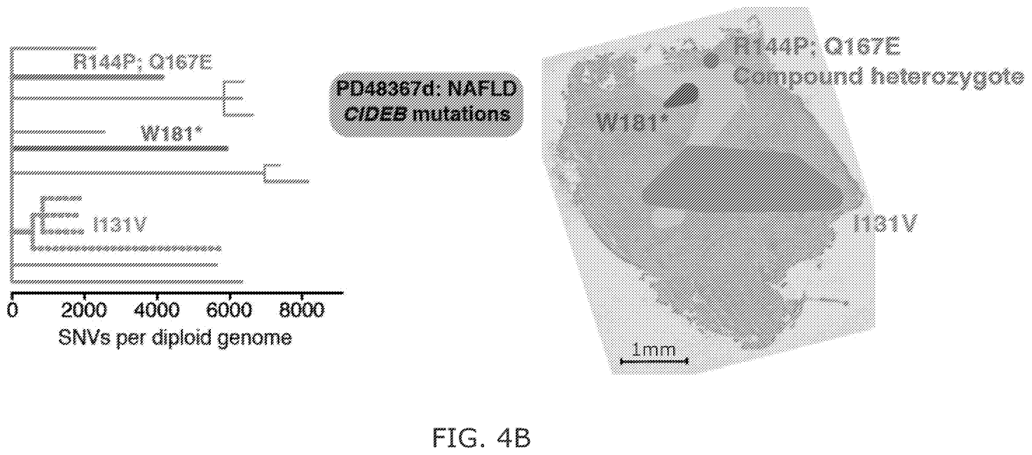

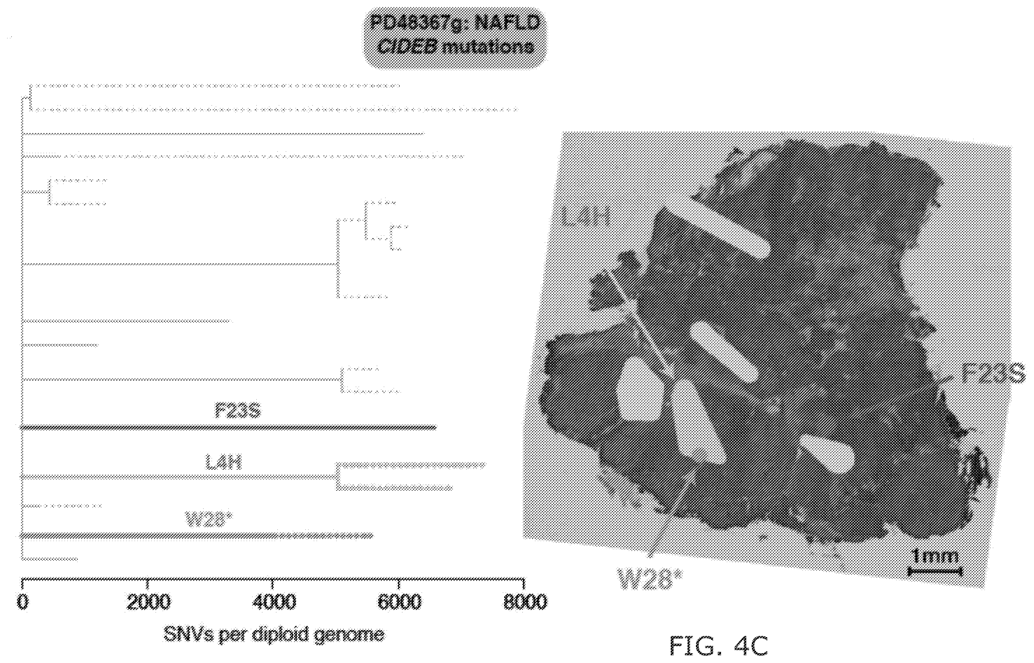

In addition to 2 nonsense and 1 stop-loss mutation, 18 missense mutations were observed in CIDEB (FIG. 4A). The missense mutations were predominantly located in the two domains implicated in homodimerisation of CIDE proteins, and many of them either switched a charged residue for a neutral one (R45W, R45Q, K140N, R144P) or even reversed the charge (D42H, K62E, E78K). Previous in vitro mutagenesis studies have shown that altering the charge on these key conserved residues, including some of those mutated in our patients, abrogates homodimerisation, preventing fusion and growth of lipid droplets within the cell (Schulze, R. J. et al., J Cell Biol 218, 2096-2112 (2019) and Lipscomb, J. C. et al., Toxicol Appl Pharmacol, 1998, 152, 376-387).

As seen for FOXO1, mutations in CIDEB were frequently acquired in multiple independent clones within one patient's liver. For example, in one of the patients with NAFLD in whom all 8 Couinaud segments of the liver were sampled, 14 clones carrying non-synonymous mutations in CIDEB were found, distributed over 6 of the 8 segments (FIG. 4B-C).

GPAM:

Another significantly mutated gene was GPAM, which encodes for mitochondrial glycerol-3-phosphate acyltransferase. This enzyme catalyses the rate-limiting step in triacylglycerol synthesis, namely the esterification of long chain acyl-CoAs with glycerol-3-phosphate (see Bergstrom, E. N. et al., BMC Genomics 20, 1-12 (2019) and (Alexandrov, L. B. et al., Nature 578, 94-101 (2020)). Compared to other glycerol-3-phosphate acyltransferase isoenzymes, GPAM is especially critical for de novo lipogenesis in the liver (Alexandrov, L. B. et al., Nature 578, 94-101 (2020)), incorporating free fatty acids synthesised from glucose into storage triglycerides. Consistent with this function, GPAM transcription is rapidly up-regulated by insulin signalling (Ericsson et al., J. Biol. Chem. 1997, 272, 7298-7305).

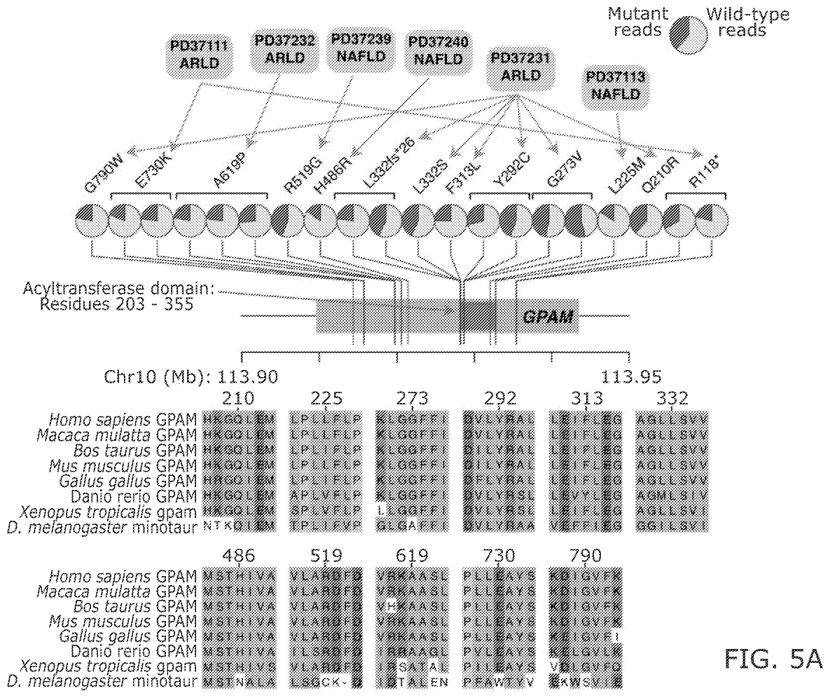

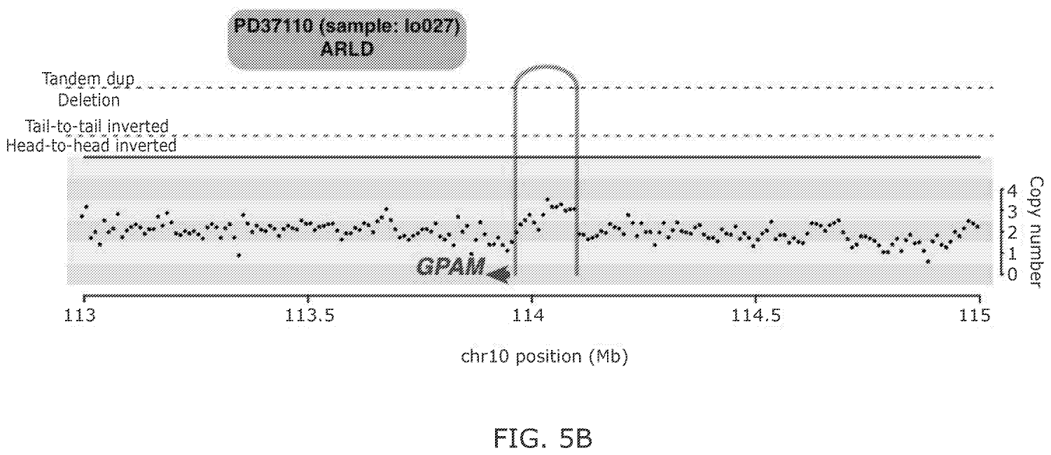

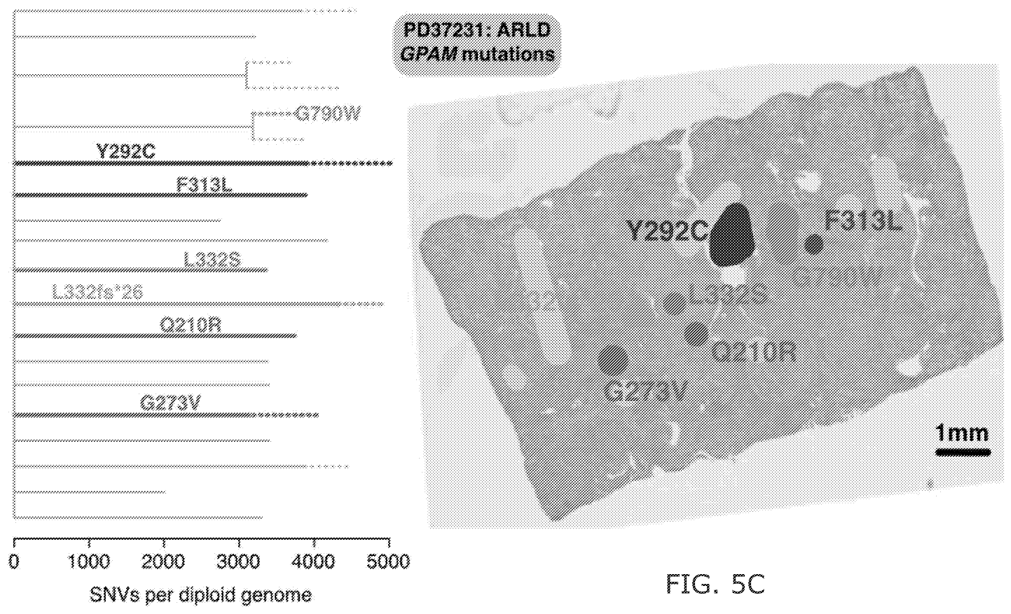

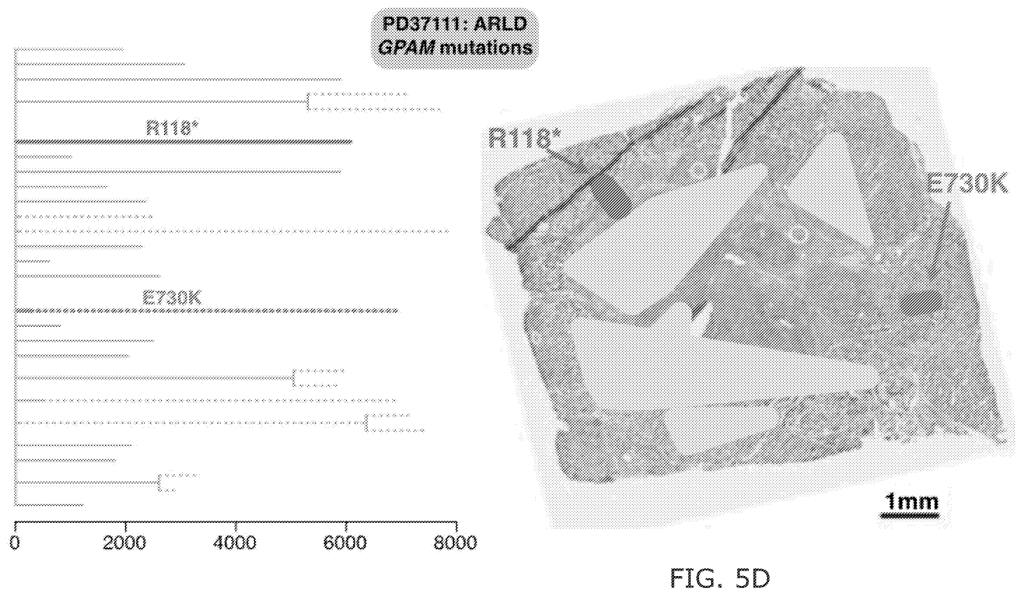

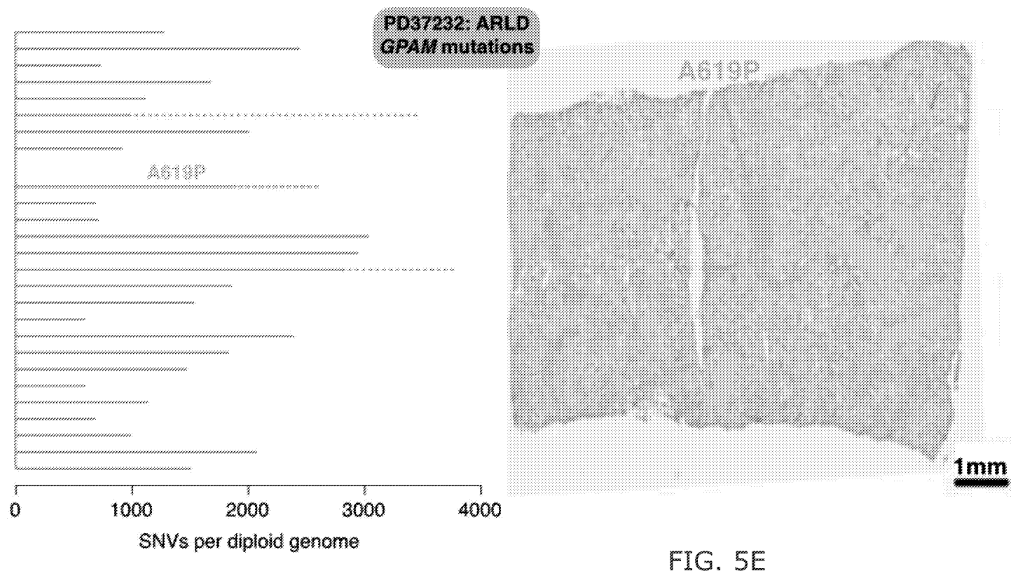

12 missense and 3 protein-truncating mutations were observed in GPAM (q=1×10⁻⁵), affecting 7 patients (FIG. 5A). A tandem duplication starting 20 kb upstream of the gene in one microdissection, of uncertain significance (FIG. 5B) was also observed. The absence of hotspot residues for mutation, coupled with the nonsense and frameshift mutations, suggests that the likely consequence of these mutations is impairment of protein function. As for FOXO1 and CIDEB mutations, there was evidence for convergent acquisition of somatic mutations in GPAM in different clones from the same patient's liver sample. For example, one patient had 7 separate events affecting GPAM (FIG. 5C), while another had 2 separate events (FIGS. 5D and 5E).

ACVR2A:

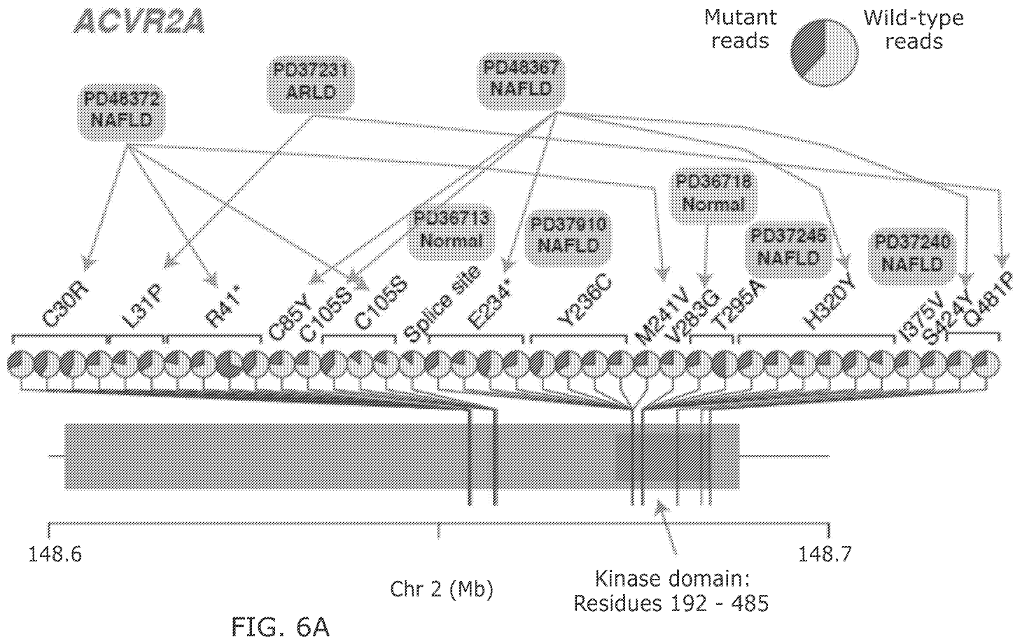

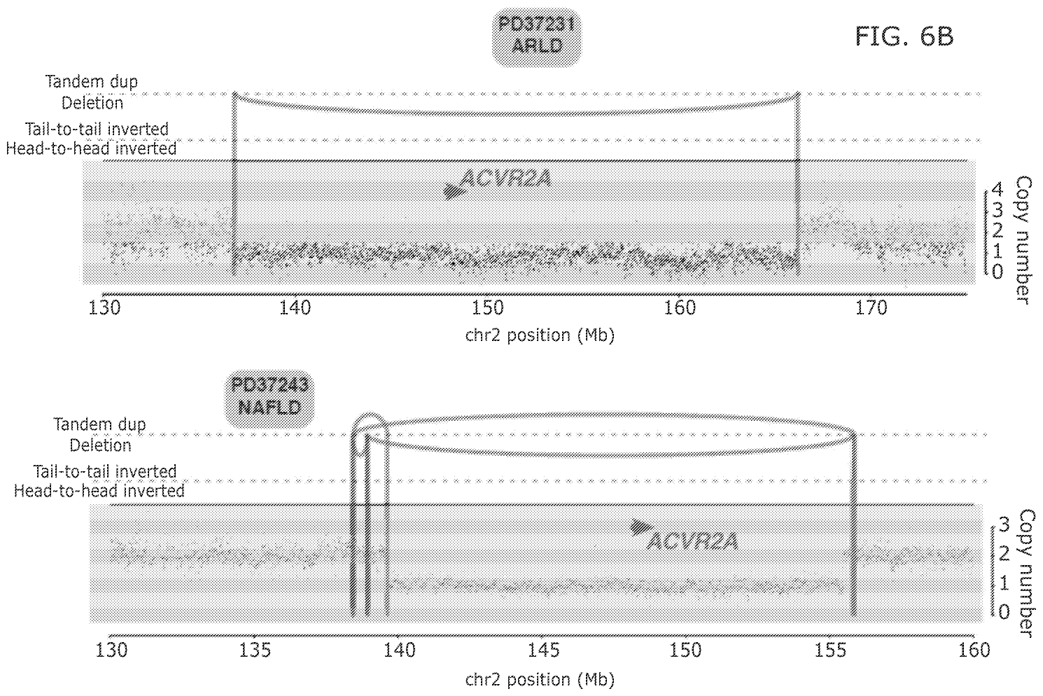

One of the four genes comprising a protein coding mutations was ACVR2A. This gene encodes a receptor for Activin-A in the TGF-β superfamily, which has been reported to be restricted to known HCC genes (Brunner et al. (2019). The ACVR2A gene is mutated in 5-10% of HCCs, with a preponderance of protein-truncating events suggesting it acts as a tumour suppressor gene. A recent meta-analysis suggests that mutations in ACVR2A are more frequently seen in cases of NAFLD- or ARLD-associated HCC than viral HCC (Chaudhary et al., Clin. Cancer Res. 2019, 25, 463-472). In the present study, 13 missense mutations, 2 nonsense and 1 splice-site indel in ACVR2A (q=7×10⁻⁹), as well as 4 large-scale deletions through structural variation were observed (FIGS. 6A and 6B). In one patient with ARLD, two independent missense point mutations and a large-scale deletion in ACVR2A in independent clones were found, consistent with convergent evolution of driver mutations within this patient.

TNRC6B:

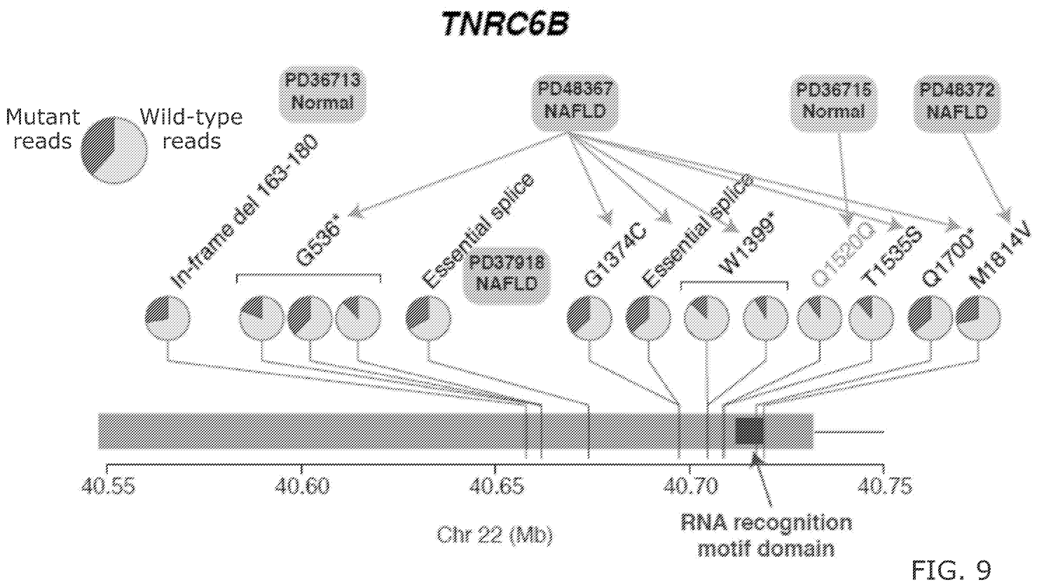

TNRC6B encodes a protein involved in microRNA processing (Meister et al. Curr. Biol. 2005, 15, 2149-2155). 3 nonsense, 2 essential splice site and one large in-frame deletion were observed, as well as 3 missense mutations in TNRC6B (q=0.04; FIG. 9). This predominance of protein-truncating variants suggests that inactivation of the gene confers a positive selective advantage on hepatocytes. Strikingly, one patient with NAFLD had 5 different mutations in TNRC6B, consistent with convergent evolution in independent hepatocyte clones.

CLCN5:

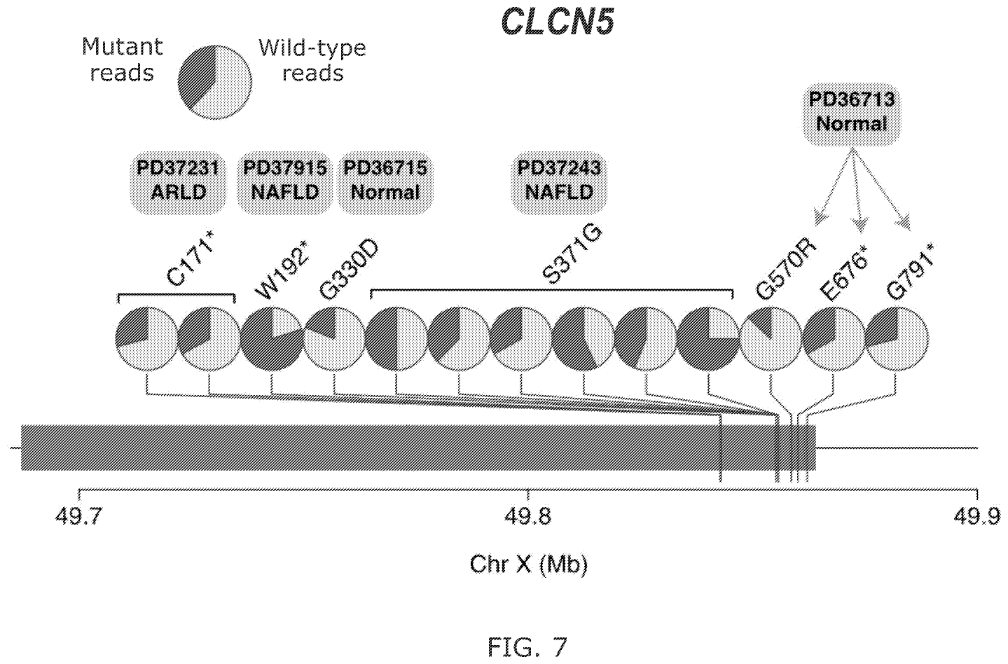

CLCN5 encodes a chloride channel, which causes X-linked nephrocalcinosis but no known liver phenotype when mutated in the germline. Three missense and 4 nonsense mutations in CLCN5 (q=0.04) were observed, distributed across patients (FIG. 7). This included two healthy controls; hence the relevance of CLCN5 mutations to chronic liver disease is uncertain.

b) Non-Coding Mutations:

A long non-coding RNA, NEAT1, showed significant excess of mutations compared to the background expectation after correction for multiple hypothesis testing (q<1×10⁻¹⁰; FIG. 8). This gene is recurrently mutated in a range of human cancers, including HCC, but this is probably because it resides within a hypermutable region of the genome rather than being under positive selection. Importantly, none of the canonical hotspot mutations or structural variants affecting the TERT promoter that are common in HCC and dysplastic nodules, whereas 11/22 (50%) of synchronous HCCs sequenced using the same protocol carried these drivers.

### Example 2: Evaluation of the FOXO1 Mutations

FOXO1 is the key transcription factor downstream of insulin signalling. In the fasting state, without insulin, FOXO1 is active in the nucleus of hepatocytes, up-regulating expression of genes in gluconeogenesis, glycolysis and lipolysis pathways. Upon insulin binding its receptor, AKT is activated through PI3K. AKT subsequently phosphorylates FOXO1 in the nucleus, with the threonine at T24 being one of three known AKT phosphorylation targets. The mutations that have been observed by the current inventors in chronic liver disease affect the R21 and S22 amino acids, which are highly conserved across evolution (FIG. 2A), and form the first two in an RS×T×P motif. When threonine T24 is phosphorylated, the motif becomes a canonical binding site for 14-3-3 proteins, which export FOXO1 from the nucleus to the cytoplasm, where the protein undergoes ubiquitination and proteasomal degradation. The S22 residue is itself phosphorylated by AMPK, inhibiting the export of FOXO1 via two mechanisms: reducing T24 phosphorylation by AKT and blocking 14-3-3 binding through steric hindrance (Saline et al. J. Biol. Chem. 2019, 294, 13106-13116). The present inventors hypothesise that the substitution of a bulky tryptophan for S22 would similarly inhibit nuclear export of FOXO1.

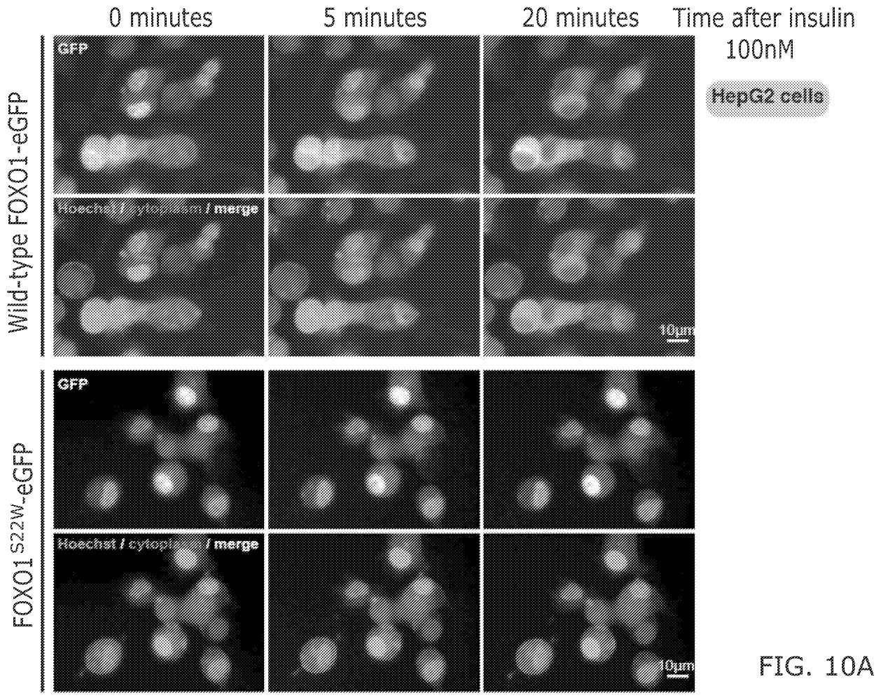

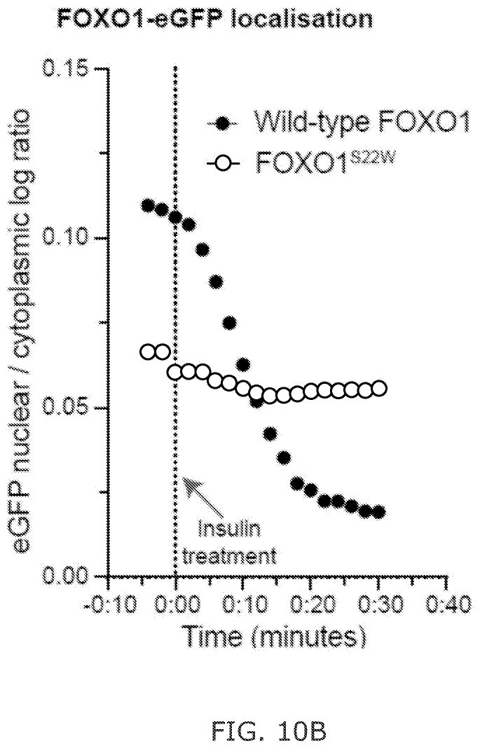

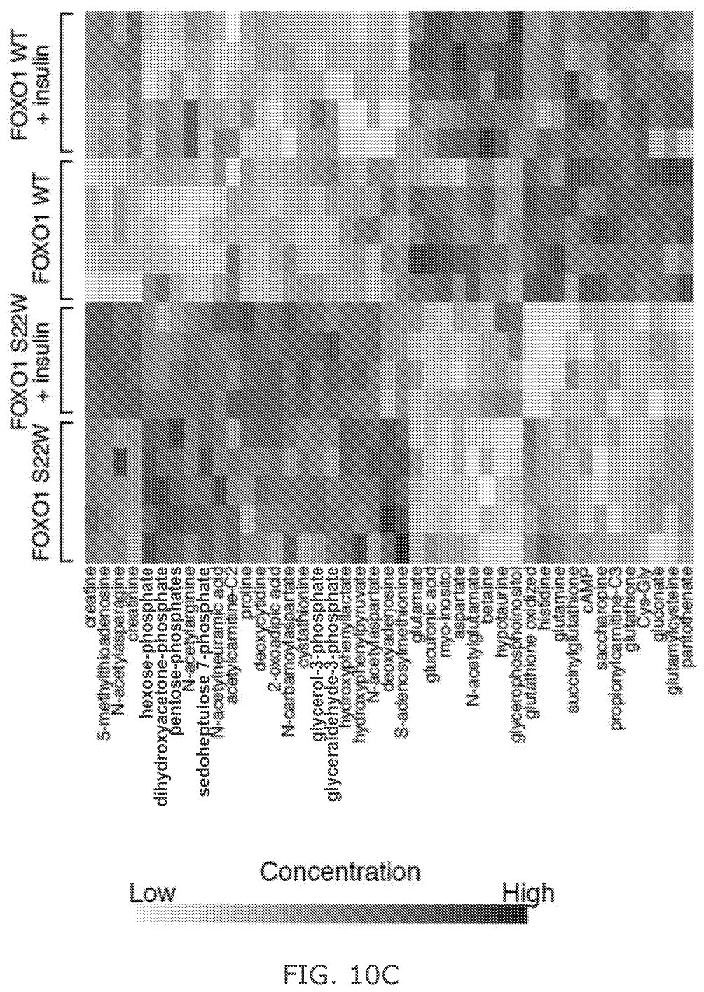

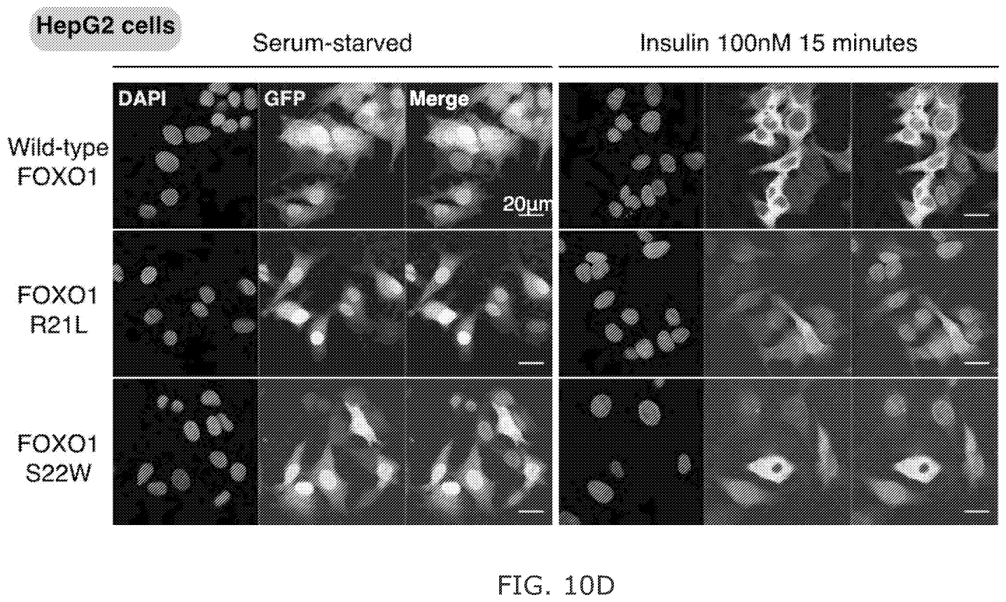

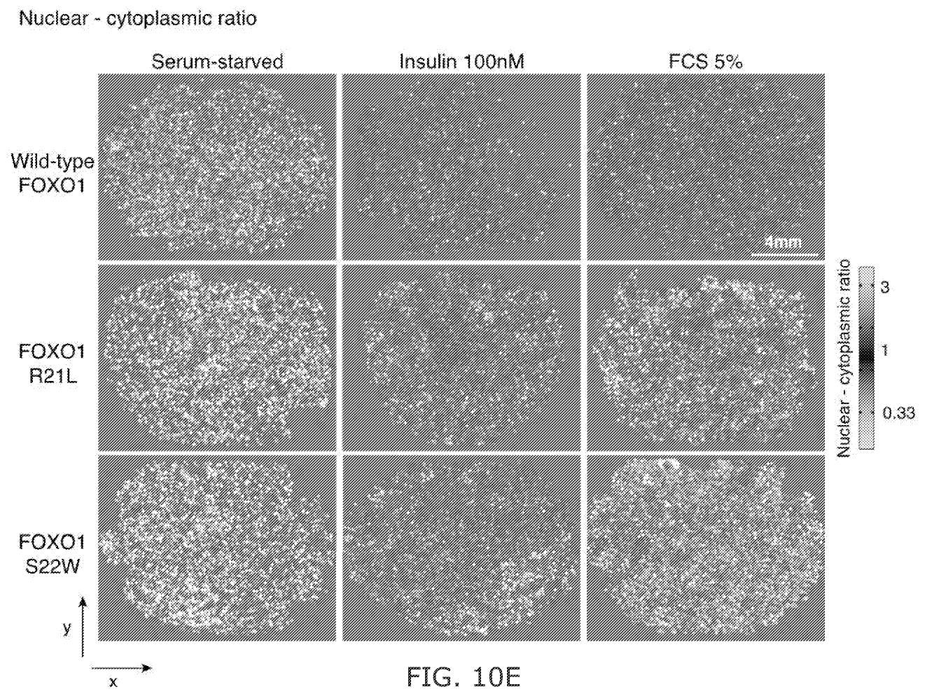

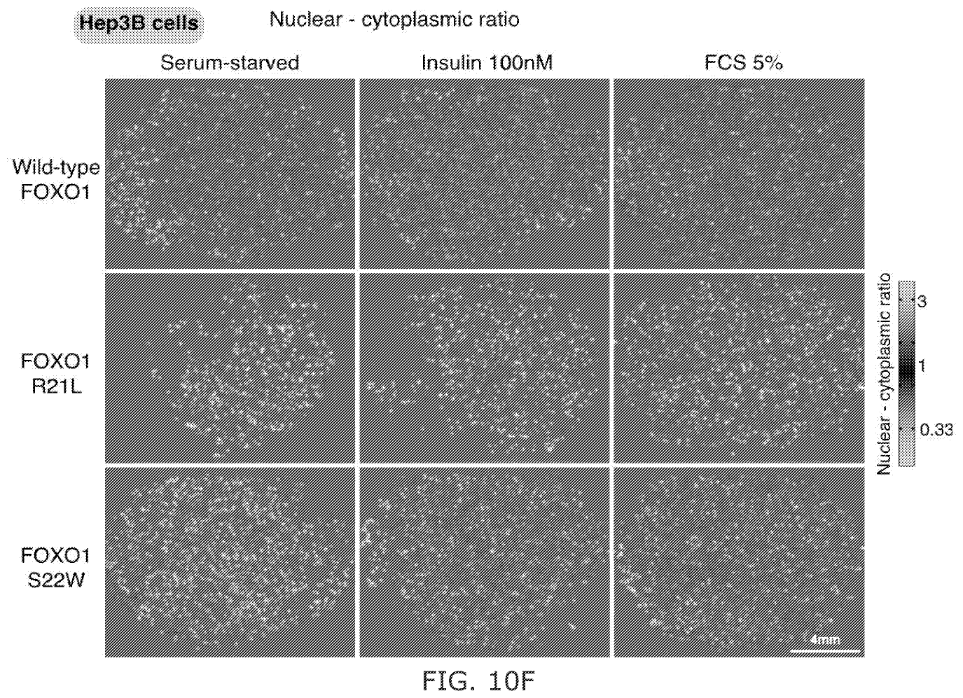

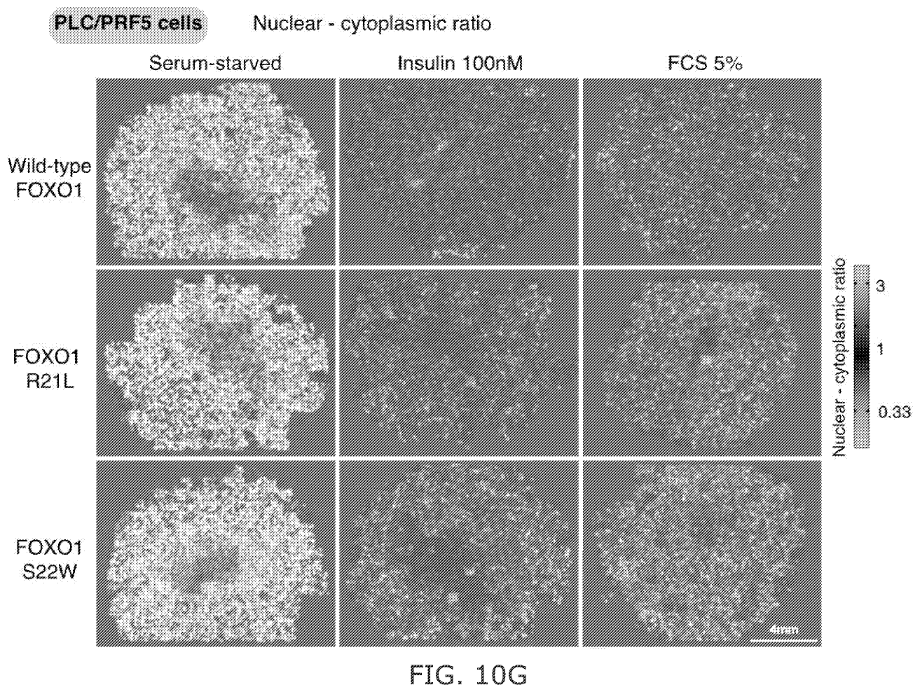

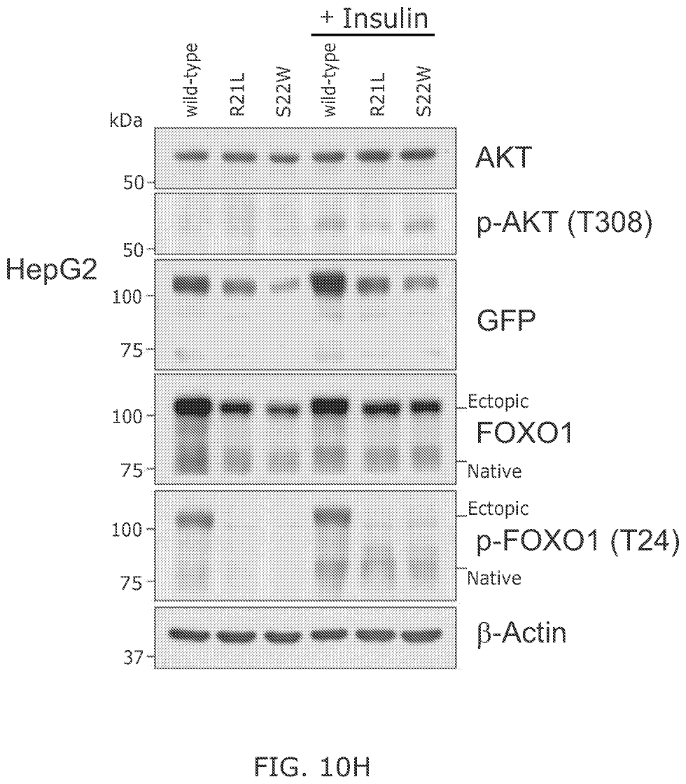

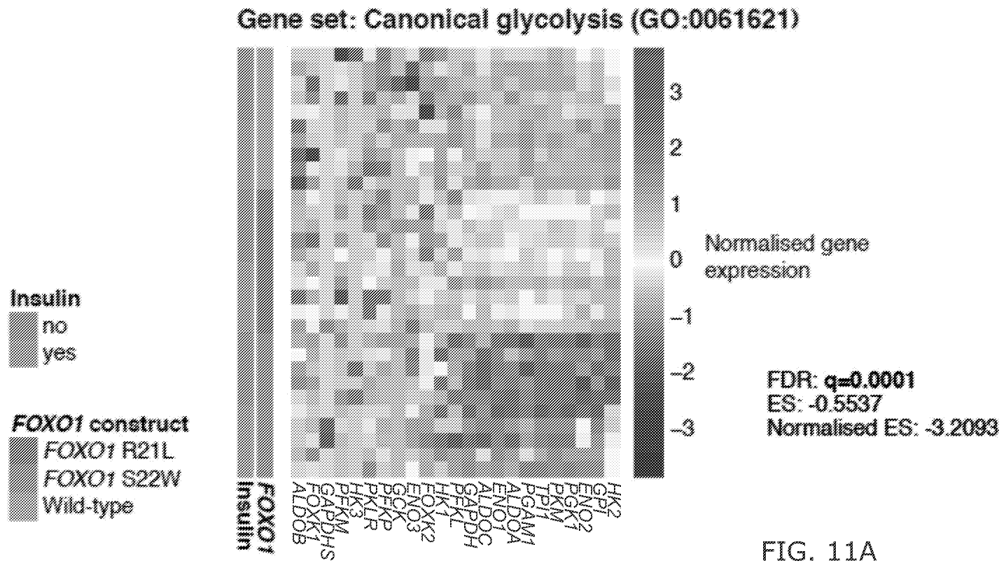

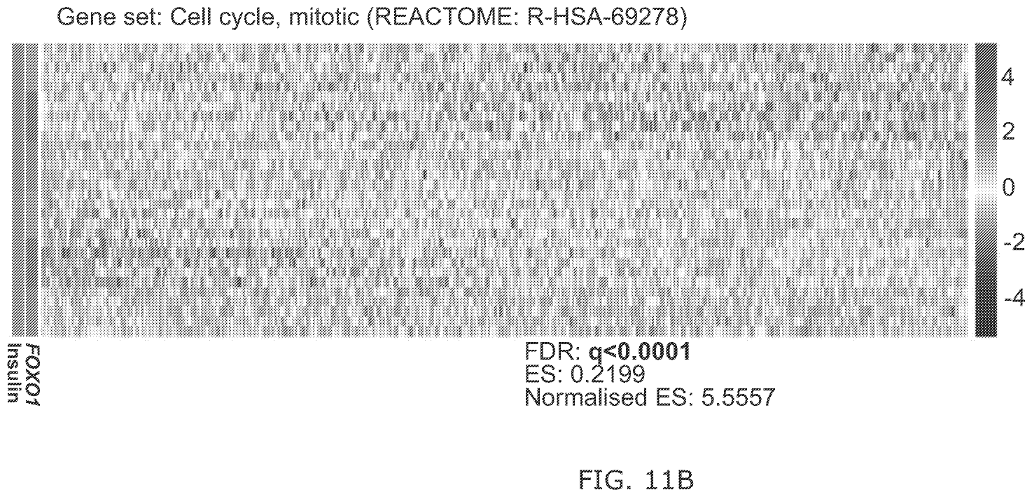

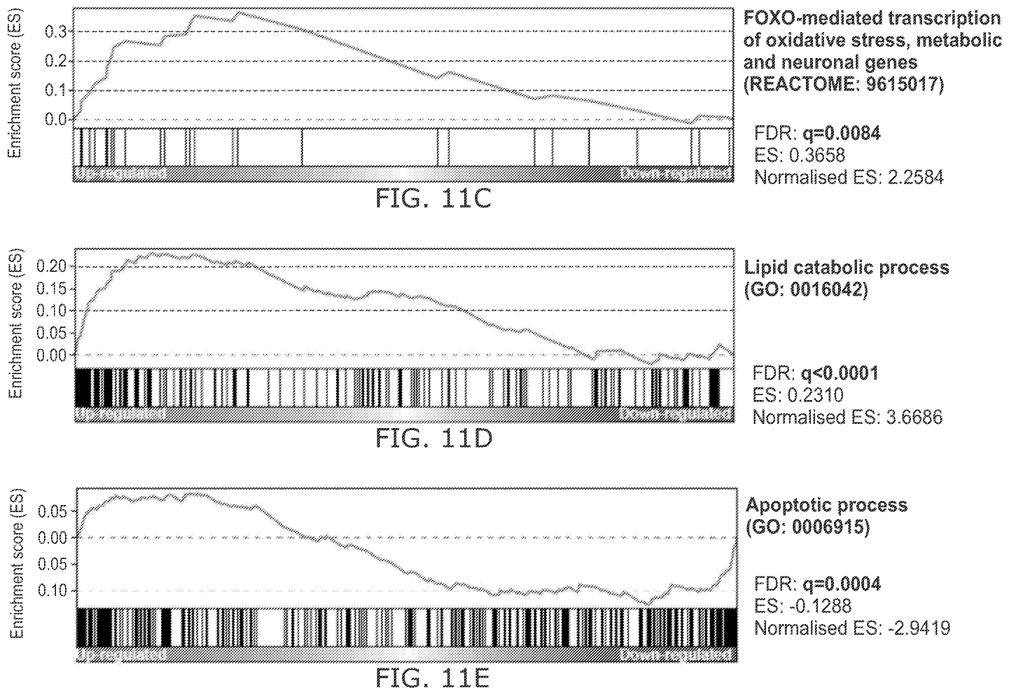

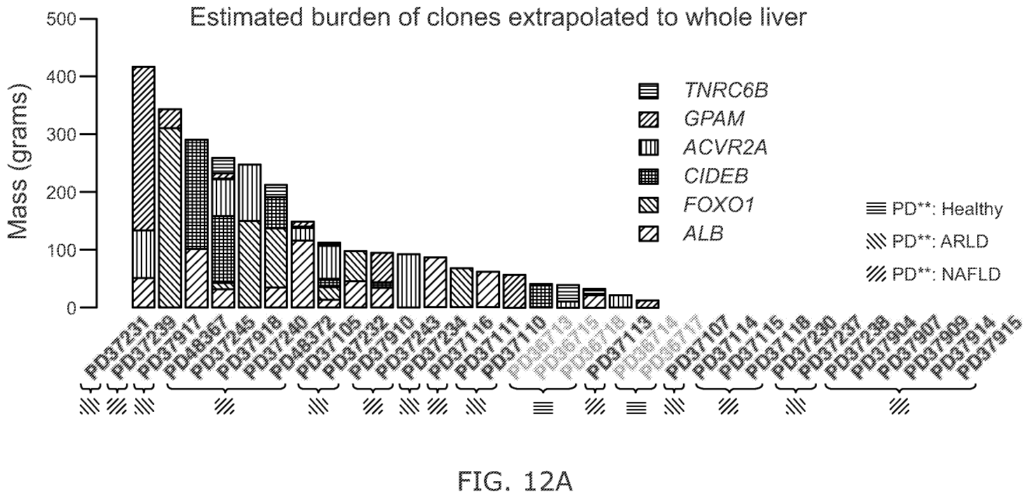

To evaluate this hypothesis, HepG2 (FIG. 10A, 10B, 10D, 10E), Hep3B (FIG. 10F) and PLC/PRF/5 (FIG. 10G) HCC cell lines were transduced with retroviral constructs of FOXO1 containing wild-type, R21L or S22W mutants, fused to C-terminal green fluorescent protein (GFP), as described below.

HepG2, Hep3B and PLC/PRF/5 cells were obtained from ATCC and cultured in Dulbecco's modified Eagle's medium (DMEM)/10% foetal calf serum (FCS) in a 5% CO₂ atmosphere. Cell identity was confirmed by STR (short tandem repeats) genotyping. Cells were regularly tested for mycoplasma contamination and always found to be negative. Insulin (Sigma) stimulation was performed by culturing the cells in serum-free DMEM for 16 hours before adding insulin for 15 minutes at a final concentration of 100 nM.

Cells were transduced with retroviral vectors (pMSCV-hFOXO1-eGFP:P2A:Puromycin) containing wild-type FOXO1 (NM_002015.4) (VB190709-1030pwk), FOXO1^{R21L} (VB190709-1028bjm) or FOXO1^{S22W} (VB190709-1032nwa), which were purchased from VectorBuilder Inc.

Results:

Under serum starvation, both wild-type and mutant FOXO1-GFP were predominantly localised to the nucleus, as expected in the absence of insulin signalling. With addition of insulin or serum, cells transduced with wild-type FOXO1-GFP showed rapid nuclear export of the protein, in keeping with the known effects of insulin. However, even in the presence of insulin or serum, cells transduced with mutant FOXO1-GFP maintained substantial levels of nuclear protein, with high nuclear-cytoplasmic ratios. An antibody to phosphorylated T24 in FOXO1 showed no binding to mutant constructs (FIG. 10H).

Levels of 105 metabolites were measured in 5 independent replicates for HepG2 cells with wild-type or S22W FOXO1-GFP, with and without insulin (FIG. 10C). Overall, 43 metabolites were significantly different between S22W and wild-type constructs, with many intermediates in glycolysis/gluconeogenesis and pentose phosphate pathways elevated in cells with mutant FOXO1-GFP, including hexose phosphates, pentose phosphates, sedoheptulose 7-phosphate, glyceraldehyde 3-phosphate and dihydroxyacetone phosphate.

RNA-sequencing of transduced HepG2 cell lines revealed significant up-regulation of gene sets involved in cell cycle (q<0.0001), lipid catabolism (q<0.0001) and FOXO-mediated transcription targets (q=0.008); and down-regulation of gene sets associated with pro-apoptotic processes (q=0.0004) and canonical glycolysis (q<0.0001) (FIGS. 10H-L). This suggests that the recurrent FOXO1 mutations seen in chronic liver disease are functional, induce changes in hepatocyte metabolism and contribute to altered hepatocyte survival.

### Example 3: Properties of Clones and Patients with Driver Mutations

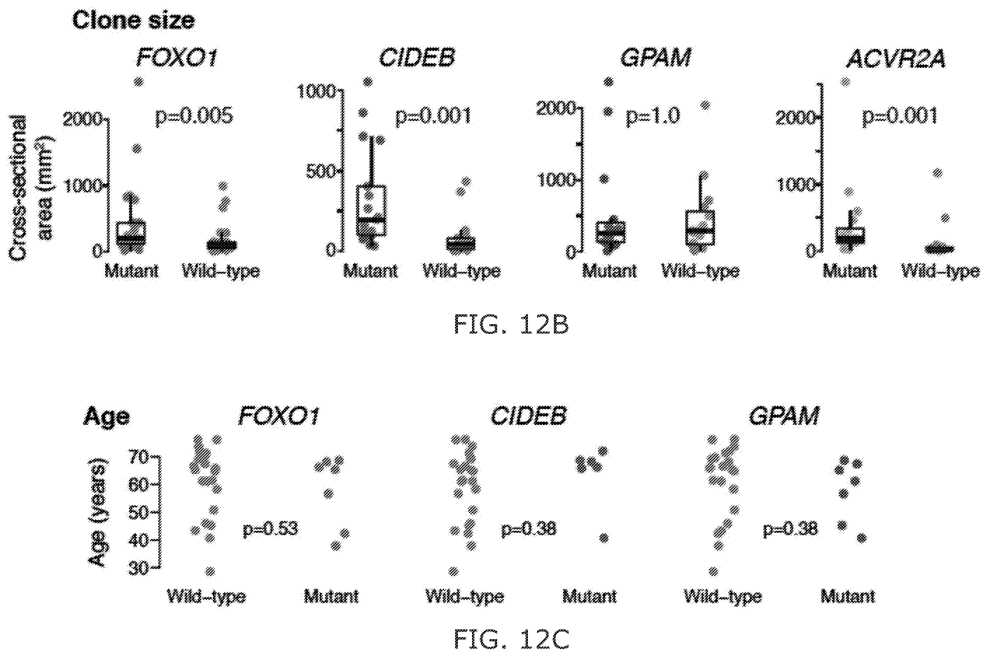

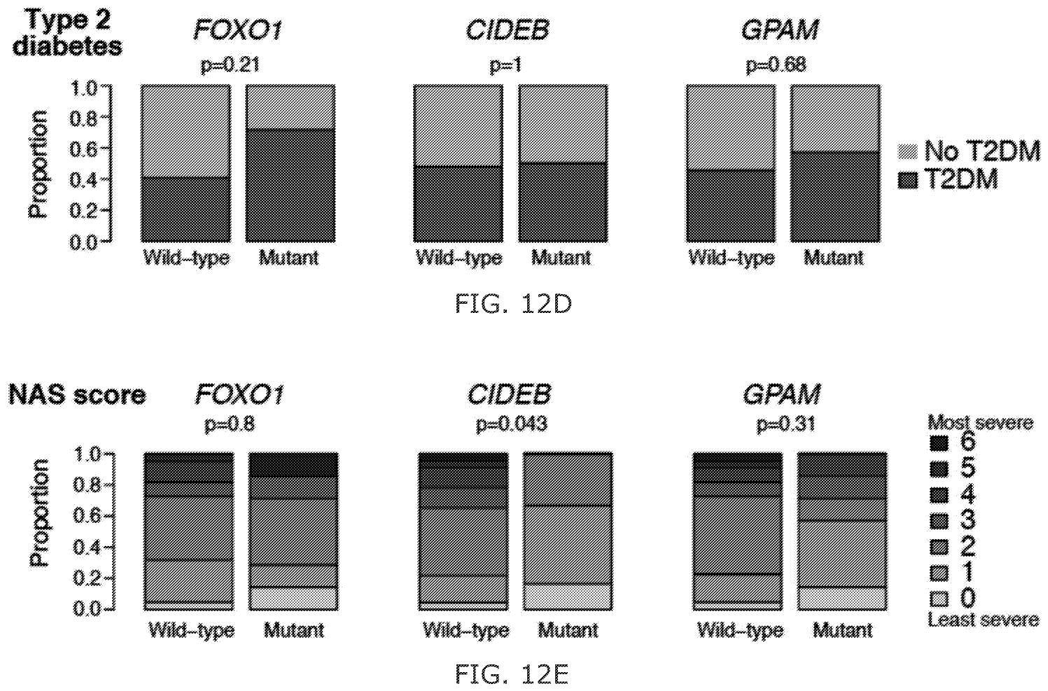

For the 2 patients in whom all 8 Couinaud segments of the liver were sampled, it was found that the driver mutations and other genomic features identified were replicated across multiple regions, suggesting that the findings from a single sample are broadly representative of the whole liver. It was therefore extrapolated from the liver samples analysed to estimate total hepatic mass carrying driver mutations in each of the significant genes (FIG. 12A). This revealed, first, that clones with driver mutations could account for hundreds of grams of liver mass in some patients, and, second, that the distribution of drivers showed considerable patient-to-patient variation in which genes were affected and what level of involvement was observed. Interestingly, the estimated sizes of clones carrying mutations in FOXO1 (p=0.005, Wilcoxon test), CIDEB (p=0.001) and ACVR2A (p=0.001) were larger on average than wild-type clones (FIG. 12B), indicating that these mutations confer a selective advantage on the clones, enabling their preferential expansion.

The S22W hotspot mutations in FOXO1, and mutations in CIDEB and GPAM, were not observed in healthy control liver samples, but were seen in liver tissue from both ARLD and NAFLD, implying their selective advantage emerges during disease development or progression. Despite the moderate cohort size, mutations in FOXO1, CIDEB and GPAM were seen across a wide range of patient characteristics: both sexes; broad age span; with and without type 2 diabetes; and variable severity of histological abnormality (FIG. 12C-E). This suggests that the results from the cohort will generalise across ARLD and NAFLD patients.

### Example 4: Comparison with Selective Landscape of HCC

To compare the selective landscape with HCC, mutation calls for 1670 HCCs were accessed from France, Japan, China and USA recorded in the ICGC data portal. As previously reported, ACVR2A is a known tumour suppressor gene in HCC (see for example The Cancer Genome Atlas Research Network. Comprehensive and Integrative Genomic Characterization of Hepatocellular Carcinoma. Cell 169, 1327-1341 (2017)), with 77 non-synonymous mutations reported in the 1670 HCCs (compared to 16 point mutations and 4 structural variants in our 1590 chronic liver disease genomes; Supplementary Tables S8-S9). TNRC6B was also significantly mutated in HCC (q=0.0001), with a predominance of protein-truncating mutations (24 variants), suggesting it is a tumour suppressor gene in HCC as well as regenerative liver from ARLD and NAFLD.

The three metabolism genes so frequently mutated in the cohort, FOXO1, CIDEB and GPAM, were not significantly mutated in HCC (q=1.0, 1.0 and 0.6 respectively). FOXO1 S22W mutations were found in only 3 of 1670 HCCs (0.18%, Cl_{95%}=0.05-0.6%), significantly lower than the 24 clones carrying this mutation in the cohort (p=2×10⁻⁵, Fisher exact test). For GPAM, the distribution of non-synonymous versus synonymous mutations was significantly different from expectation upon testing as a single gene (p=0.006), but no longer significant with correction for multiple hypothesis testing (q=0.6).

### Example 5: Telomere Lengths, and Heritability

A large majority of HCCs have mutations activating TERT, the telomerase gene as reported in Schulze, K. et al. Nat. Genet. 47, 505-511 (2015), Fujimoto, A. et al. Nat. Genet. 44, 760-4 (2012), Fujimoto, A. et al. Nat. Genet. 48, 500-509 (2016), and Li, Y. et al. Nature 578, 112-121 (2020). However, in the regenerative nodules studied here, only one mutation affecting the canonical hotspots in the TERT promoter was observed. To assess telomere dynamics in the samples, the telomere length for each microdissection (Farmery, J. H. R. et al. Sci. Rep. 2018, 8, 1-17) was estimated.

The telomere length (in units of base-pairs) of each microdissection studied was estimated by analysing the corresponding WGS data for telomeric reads (containing TTAGGG and CCCTAA hexamers). To accomplish this, Telomerecat v3.4.0 software was used (Farmery et al. (2019)), with length correction enabled, while setting the number of simulations to 100 to constrain uncertainties in the length estimates. Each SNV cluster was assigned the telomere length corresponding to the member microdissection with the highest median VAF.

Telomere lengths were modelled using Bayesian mixed effects models—these enabled us to assess the effects of age, clone size and disease on telomere lengths, while concurrently controlling for and quantifying the correlation arising from phylogenetic relationships among clones and within-patient non-independence. The specific algorithm used was the R package, MCMCgImm (Hadfield, J. Stat. Softw. 2010, 33, 1-22).

To use these models effectively, a relatedness matrix among clones on phylogenetic trees was constructed. This required making trees ultrametric (namely, all tips finish at the same point). Although there are existing methods for making trees ultrametric, they did not especially suit the current purposes for somatic mutations in normal tissues. The current inventors established more power for estimating the true number of early mutations (since they will be seen in more than one microdissection, and on average at higher variant allele fraction). The estimates of branch length for the late, singleton branches found by the current inventors will be less certain. Therefore, the current inventors developed a recursive function to place each branch-point (coalescence) on a given fraction of molecular time. Starting at the root of the tree, and progressing towards the tip, the position of each coalescence is estimated as the number of mutations acquired between root and that coalescence divided by that number plus the average of the number of mutations in branches descending from the coalescence.

A Bayesian linear mixed effects model was used to model telomere lengths. The dependent variable was the average telomere length (‘Length’) of each clone, measured in base-pairs, and expected to follow a Gaussian distribution. The fixed effects were: Aetiology (dummy variables for ARLD and NAFLD); Age of patient (in years); Number of mutations in that clone; and Clone area. The current inventors ran fitted a random effect for each patient and a random effect for the phylogenetic relationships encoded in the form of a block diagonal matrix. The priors were uninformative inverse-Wishart distributions. The MCMC chain was run for 11,000,000 iterations with 1,000,000 of these as a burn-in, thinned to every 1000 iterations.

The usual diagnostics on the MCMC chain were checked—these showed (1) there were no systematic trends in the variables after the burn-in has completed (the burn-in was of sufficient length); (2) autocorrelation was relatively low among adjacent estimates with the thinning to every 1000th instance of the chain; (3) diagnostic tests of convergence were all passed.

Estimates for the ‘somatic heritability’ of telomere lengths were derived by calculating the component of variance in the random effects that was attributed to the variable describing phylogenetic relationships versus the residual variance and variance attributed to between-patient effects. The estimate of ‘somatic heritability’ for telomere lengths was 21% (95% posterior interval=0-44%)— that is, once the effects of age, disease and clone size have been accounted for, ˜21% of the residual variation in telomere lengths across the cohort can be explained by the phylogenetic relationships among clones. However, there is some instability in this estimate, and maybe about 26% of iterations of the MCMC chain suggest no somatic heritability. Either way, the confounding effects of this between-sample correlation have been corrected within the modelling framework.

Results:

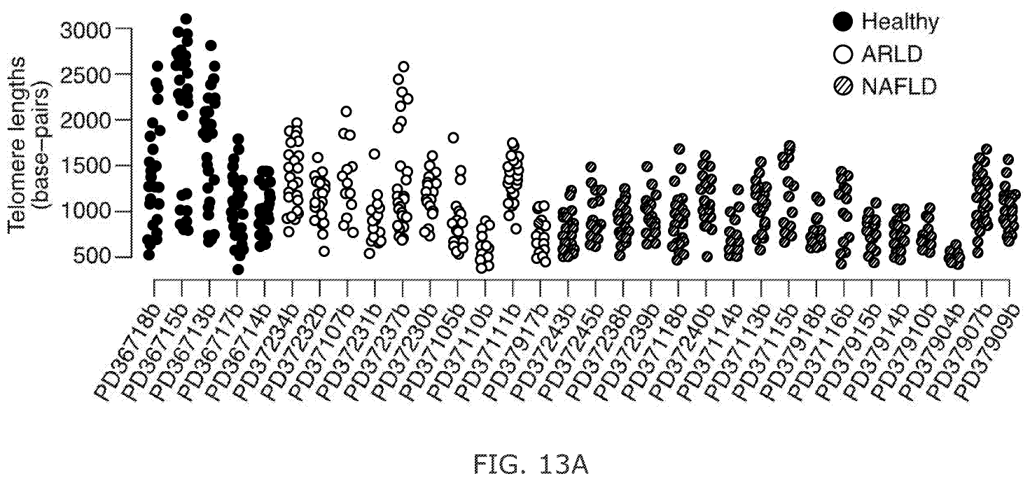

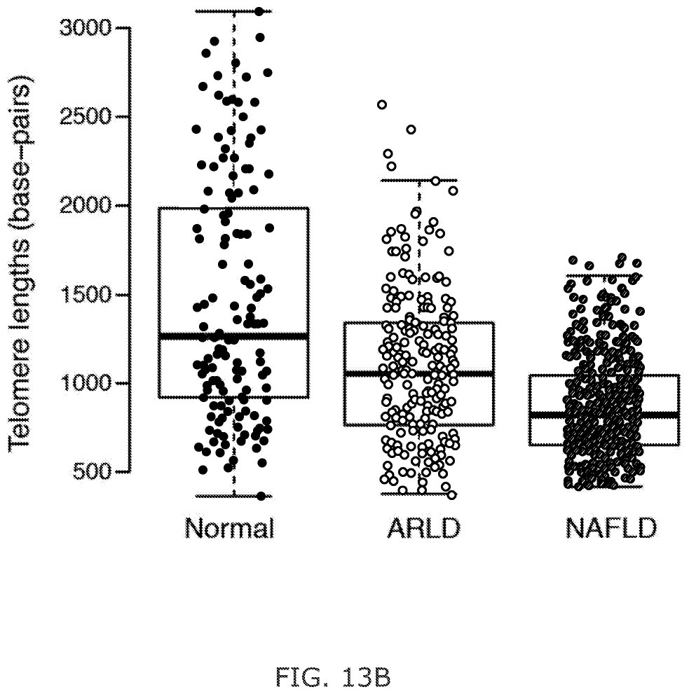

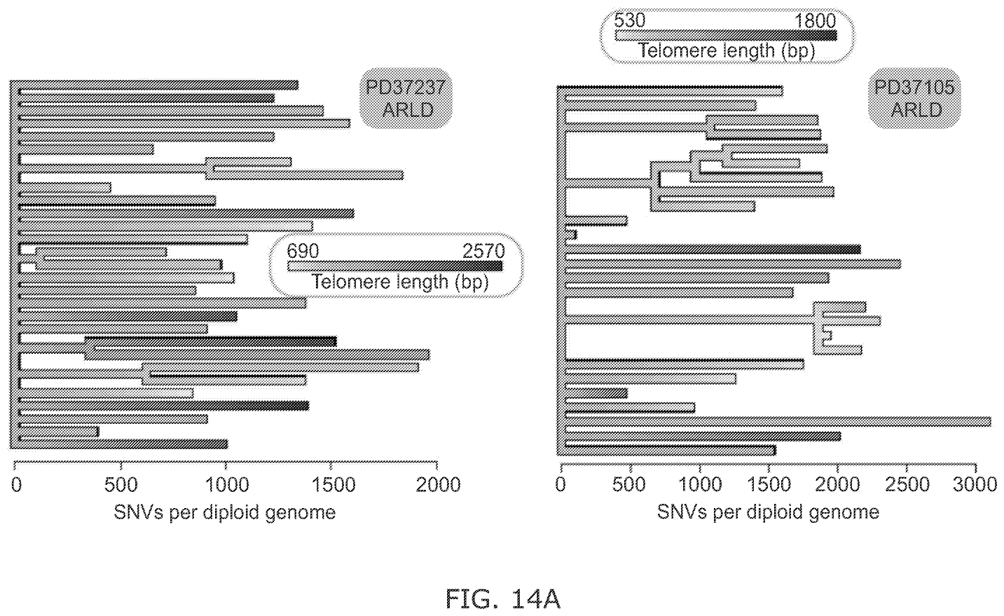

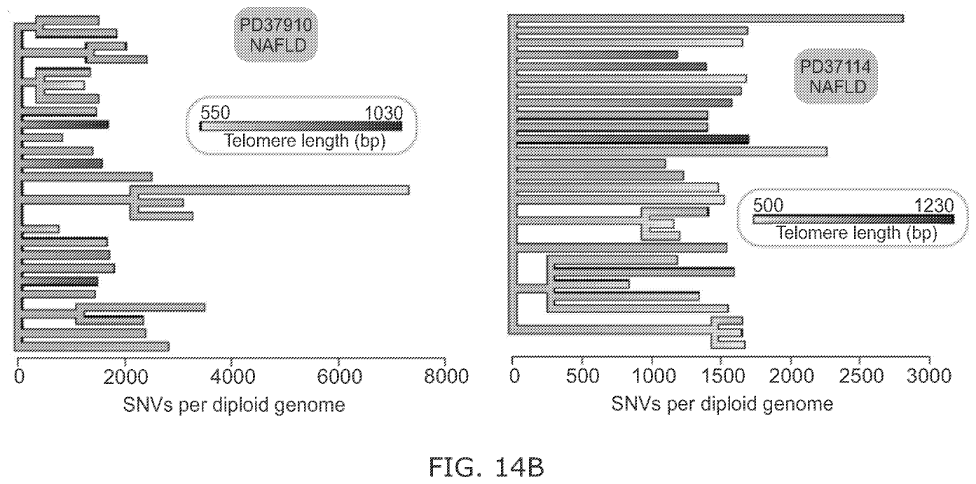

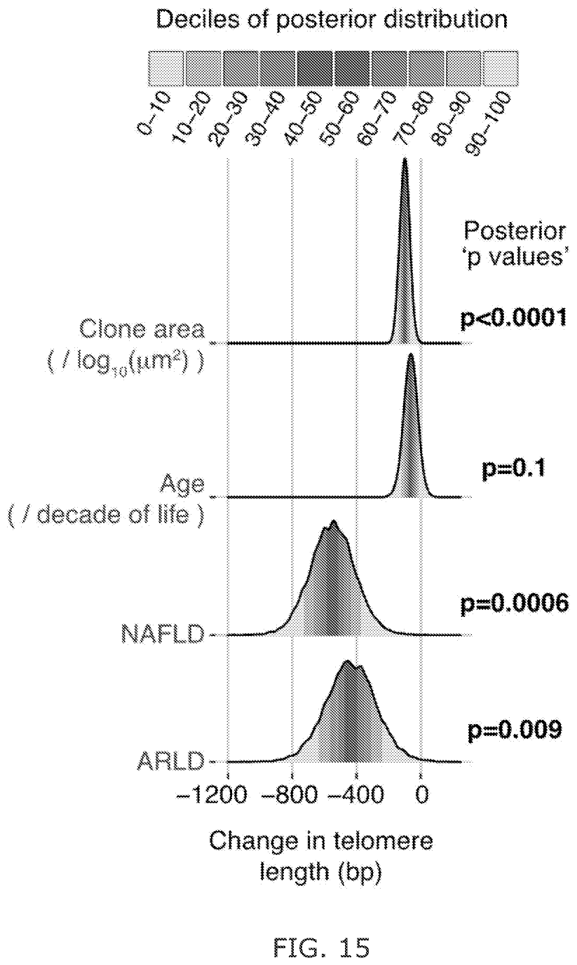

Telomere lengths for each microdissection from the whole genome sequencing data were estimated. Considerable between- and within-individual variation was observed in telomere lengths across the cohort, with apparently shorter telomeres in NAFLD and ARLD compared to normal liver (FIGS. 13A, 13B and B15. Layering these telomere lengths onto phylogenetic trees revealed that, on average, more closely related clones had more similar telomere lengths than unrelated clones (FIGS. 14A and 14B).

Telomere lengths using Bayesian mixed effects models were modelled, which enabled the assessment of the effects of age, clone size and disease on telomere lengths, while concurrently controlling for correlation arising from phylogenetic relationships among clones. Compared to normal liver, telomere lengths were considerably shorter in patients with ARLD (mean_{Posterior}=−440 bp; 95% posterior interval (Pl_{95%})=−750:−130 bp; p_{Posterior}=0.009) and NAFLD (mean_{Posterior}=−550 bp; Pl_{95%}=−820:−270 bp; p_{Posterior}=0.0006; FIG. 12C). These effect sizes for disease considerably outweighed the relatively minor shortening of telomere lengths with age (mean_{Posterior}=−6 bp/decade; Pl_{95%}=−15:+2 bp/decade; p_{Posterior}=0.1). Furthermore, telomeres became progressively shorter as the size of a clone increased (mean_{Posterior}=−100 bp/log₁₀(μm²); Pl_{95%}=−160:−50 bp/log₁₀(μm²); p_{Posterior}<0.0001).

These data suggest that ARLD and NAFLD are associated with substantial attrition of telomeres, outweighing the relatively minor shortening of telomere lengths with age. Furthermore, telomeres became progressively shorter as the size of a clone increased, presumably reflecting the extra cell divisions associated with hepatocyte regeneration during disease progression.

1. A method for diagnosing and/or prognostication of non-alcoholic fatty liver disease (NAFLD) or alcohol-related fatty liver disease (ARLD) in a subject, said method comprising:
a) providing a biological sample comprising DNA, RNA and/or protein derived from one or more liver cells of the subject;
b) detecting a somatic mutation in the DNA, RNA and/or protein that confers a selective advantage on the liver cell, wherein the presence of a somatic mutation that confers a selective advantage on the liver cell indicates that the subject is suffering from NAFLD or ARLD, is at risk of developing NAFLD or ARLD, is at risk of developing a more severe form of liver disease, and/or is at risk of developing a disease or condition associated with liver disease.

2. The method as claimed in any one of the preceding claims, wherein the somatic mutation detected in step b) is within a gene selected from group consisting of FOXO1, GPAM, CIDEB, ACVR2A, ALB and TNRC6B.

3. The method as claimed in any one of the preceding claims, wherein the somatic mutation detected in step b) is within a gene selected from group consisting of FOXO1, GPAM, and CIDEB.

4. The method as claimed in any one of the preceding claims, wherein the somatic mutation detected in step b) is within the FOXO1 gene.

5. The method as claimed in claim 4, wherein the somatic mutation within the FOXO1 gene result in a missense and/or nonsense mutation within the N-terminal 14-3-3 protein binding motif of a FOXO1 protein expressed by the one or more liver cells.

6. The method as claimed in claim 4 or 5, wherein the somatic mutation within the FOXO1 gene result in a S22W or R21L amino acid substitution or a S22 nonsense mutation in a FOXO1 protein expressed by the one or more liver cells.

7. The method as claimed in claim 6, wherein the somatic mutation within the FOXO1 gene results in a S22W amino acid amino acid substitution in a FOXO1 protein expressed by the one or more liver cells.

8. The method as claimed in any one of claims 1 to 3, wherein the somatic mutation detected in step b) is within the GPAM gene.

9. The method as claimed in claim 8, wherein the somatic mutation within the GPAM gene impairs or abrogates the function of mitochondrial glycerol-3-phosphate acyltransferase (GPAT) expressed by the one or more liver cells.

10. The method as claimed in any one of claims 1 to 3, wherein the somatic mutation detected in step b) is within the CIDEB gene.

11. The method as claimed in claim 10, wherein the somatic mutation within the CIDEB gene impairs or abrogates the function of a CIDEB protein expressed by the one or more liver cells.

12. The method as claimed in any one of the preceding claims, wherein step b) further comprises a step of detecting a somatic mutation within the CLCN5 gene.

13. The method as claimed in any one of the preceding claims, wherein the biological sample comprises circulating free DNA (cfDNA).

14. The method of any preceding claim, wherein the biological sample comprises DNA, RNA and/or protein derived from a liver biopsy sample.

15. The method as claimed in any one of the preceding claims, wherein the biological sample comprises DNA, RNA and/or protein derived from one or more hepatocyte cells.

16. The method as claimed in any one of the preceding claims, wherein step b) comprises determining if the subject is suffering from NAFLD or ARLD, is at risk of developing NAFLD or ARLD, and/or is at risk of developing a more severe form of liver disease selected from the group consisting of non-alcoholic steatohepatitis (NASH), liver fibrosis, liver cirrhosis, cancer (for example hepatocellular carcinoma (HCC)) and liver failure.

17. The method as claimed in any one of the preceding claims, wherein step b) comprises determining if the subject is at risk of developing a disease or condition associated with liver disease selected from the group consisting of gastrointestinal cancer, obesity, type 2 diabetes mellitus, hypertension, dyslipidaemia (e.g. hypercholesterolaemia) and cardiovascular disease.

18. The method as claimed in any one of claims 1 to 16, wherein the subject is one who is also suffering from gastrointestinal cancer, obesity, type 2 diabetes mellitus, hypertension, dyslipidaemia (e.g. hypercholesterolaemia) and/or cardiovascular disease.

19. The method as claimed in any preceding claim, wherein step a) comprises providing a biological sample comprising DNA, RNA and/or protein derived from two or more liver cells of the subject, for example from about 10 to about 50,000 liver cells of the subject.

20. The method of any preceding claim, wherein the method comprises:
i) providing a biological sample obtained from the subject; and
ii) detecting one or more somatic mutations in the FOXO1 and/or GPAM genes in the biological sample, wherein the presence of one or more somatic mutations indicates that the subject is suffering from NAFLD, is at risk of developing NAFLD, is at risk of developing a more severe form of liver disease, and/or is at risk of developing a disease or condition associated with liver disease.

21. A method for identifying a subject suffering from NAFLD or ARLD who would benefit from increased disease monitoring, said method comprising the method steps of any one of claims 1 to 20.

22. A method for identifying a subject suffering from NAFLD or ARLD who would benefit from treatment with a therapeutic agent that inhibits or modulates FOXO1, CIDEB, ACVR2A, GPAM or TNRC6B protein activity in the liver, said method comprising the method steps of any one of claims 1 to 20.

23. A therapeutic agent for use in the treatment of NAFLD or ARLD, wherein said use comprises
performing the method of any one of claims 1 to 20; and
administering to the subject the therapeutic agent, wherein the therapeutic agent is one that inhibits or modulates FOXO1, CIDEB, ACVR2A, GPAM or TNRC6B protein activity.

24. A method of treating a subject suffering from NAFLD or ARLD, said method comprising
performing the method of any one of claims 1 to 20; and
administering to the subject a dose of a therapeutic agent that inhibits or modulates FOXO1, CIDEB, ACVR2A, GPAM or TNRC6B protein activity.

25. The use according to claim 23, or the method of claim 24, wherein the therapeutic agent is selected from the group consisting of small molecules, peptides (including cyclic peptides), proteins (e.g. therapeutic antibodies, antibody fragments and antibody mimetics), nucleic acids (e.g., DNA and RNA nucleotides including, but not limited to, antisense nucleotide sequences, triple helices, siRNA or miRNA, and nucleotide sequences encoding biologically active proteins, polypeptides or peptides), synthetic or natural inorganic molecules, synthetic or natural organic molecules, and CRISPR.

26. An in vitro diagnostic kit for use in the diagnosis or prognosis of NAFLD or ARLD in a subject according to the method of any one of claims 1 to 20, said kit comprising one or more reagents for detecting one or more somatic mutations within a gene selected from the group consisting of FOXO1, CIDEB, ACVR2A, ALB, GPAM and TNRC6B, and optionally detecting one or more somatic mutations within the CLCN5 gene or the NEAT1 gene, and/or measuring telomere length.

27. A method for diagnosing or prognostication of NAFLD or ARLD in a subject, said method comprising the steps of
administering a dose of a diagnostic probe to the subject; and
detecting the diagnostic probe in the subject, wherein said diagnostic probe indicates the presence and/or absence of one or more somatic mutations within a gene selected from the group consisting of FOXO1, CIDEB, ACVR2A, ALB, GPAM and TNRC6B of the subject, and wherein the presence of one or more somatic mutations within a gene selected from the group consisting of FOXO1, CIDEB, ACVR2A, ALB, GPAM and TNRC6B indicates that the subject is suffering from NAFLD or ARLD, is at risk of developing NAFLD or ARLD, is at risk of developing a more severe form of liver disease and/or is at risk of developing a disease or condition associated with liver disease such as gastrointestinal cancer.
